(19) United States
(12) Patent Application Publication
(10) Pub. No.: US 2008/0051392 A1
(43) Pub. Date: Feb. 28, 2008

(54) PHARMACEUTICAL COMBINATIONS FOR THE TREATMENT OF RESPIRATORY DISEASES

(76) Inventors: Ingo Konetzki, Warthausen (DE); Thierry Bouyssou, Warthausen (DE); Sabine Pestel, Attenweiler (DE); Andreas Schnapp, Biberach (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(21) Appl. No.: 11/842,480

(22) Filed: Aug. 21, 2007

(30) Foreign Application Priority Data
Aug. 22, 2006 (EP) 06119272.0

(51) Int. Cl. A61K 31/535; A61P 11/00

(52) U.S. Cl. 514/230.5

(57) ABSTRACT

The present invention relates to new pharmaceutical combinations which contain in addition to one or more, preferably one compound of general formula 1 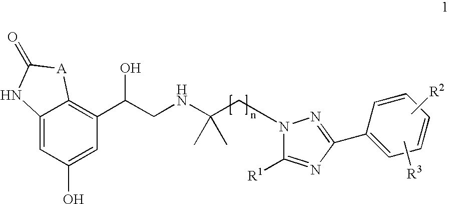
wherein the groups R¹, R² and R³ may have the meanings given in the claims and specification, at least one further active substance 2, processes for preparing them and their use as medicaments.

This application claims priority benefit from EP 06 119 272.0, filed Aug. 22, 2006, which is incorporated herein in its entirety.

The present invention relates to new pharmaceutical combinations which contain in addition to one or more, preferably one compound of general formula 1 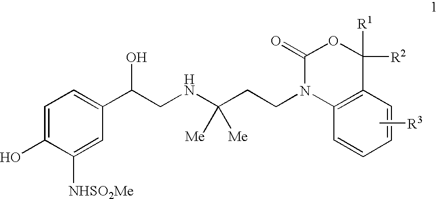
wherein the groups R¹, R² and R³ may have the meanings given in the claims and specification, at least one further active substance 2, methods of preparing them and their use as medicaments.

### DETAILED DESCRIPTION OF THE INVENTION

The present invention relates to pharmaceutical combinations which contain in addition to one or more, preferably one compound of general formula 1 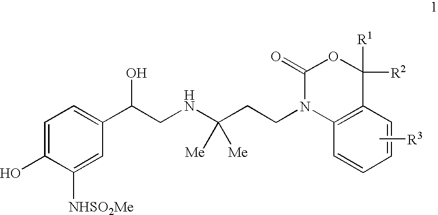
wherein
R¹ and R² independently of one another denote H, halogen or C₁₋₄-alkyl or together denote C₁₋₆-alkylene; and
R³ denotes H, halogen, OH, C₁₋₄-alkyl or O—C₁₋₄-alkyl;
   optionally in the form of the pharmaceutically acceptable acid addition salts, hydrates or solvates thereof, at least one further active substance 2.

Preferably the present invention relates to pharmaceutical combinations which contain in addition to one or more, preferably one compound of formula 1 as a further active substance 2 one or more compounds, which are selected from among the anticholinergics (2a), PDE-IV-inhibitors (2b), steroids (2c), LTD4-antagonists (2d) and EGFR-inhibitors (2e).

Preferred pharmaceutical combinations as defined above are those which contain in addition to one or more, preferably one compound of general formula 1, wherein
R¹ and R² which may be identical or different, denote hydrogen, fluorine, chlorine, methyl, ethyl, propyl, butyl or together denote —CH₂—CH₂, —CH₂—CH₂—CH₂, —CH₂—CH₂—CH₂—CH₂ or —CH₂—CH₂—CH₂—CH₂—CH₂—;
R³ denotes hydrogen, fluorine, chlorine, OH, methyl, ethyl, methoxy, or ethoxy
   optionally in the form of the pharmaceutically acceptable acid addition salts, hydrates or solvates thereof, at least one further active substance 2.

Preferred pharmaceutical combinations as defined above are those which contain in addition to one or more, preferably one compound of general formula 1, wherein
R¹ and R² which may be identical or different, denote hydrogen, methyl, ethyl, propyl or together denote —CH₂—CH₂, —CH₂—CH₂—CH₂, —CH₂—CH₂—CH₂—CH₂ or —CH₂—CH₂—CH₂—CH₂—CH₂—;
R³ denotes hydrogen, fluorine, OH, methyl or methoxy;
   optionally in the form of the pharmaceutically acceptable acid addition salts, hydrates or solvates thereof, at least one further active substance 2.

Also preferred are the above pharmaceutical combinations which contain in addition to one or more, preferably one compound of general formula 1, wherein
R¹ and R² which may be identical or different, denote ethyl, propyl or together denote —CH₂—CH₂, —CH₂—CH₂—CH₂, —CH₂—CH₂—CH₂—CH₂ or —CH₂—CH₂—CH₂—CH₂—CH₂—;
R³ denotes hydrogen, fluorine, OH, methyl or methoxy;
   optionally in the form of the pharmaceutically acceptable acid addition salts, hydrates or solvates thereof, at least one further active substance 2.

Also preferred are the above pharmaceutical combinations which contain, in addition to one or more, preferably one compound of general formula 1, wherein
R¹ and R² denote ethyl, propyl or together denote —CH₂—CH₂, —CH₂—CH₂—CH₂, —CH₂—CH₂—CH₂—CH₂ or —CH₂—CH₂—CH₂—CH₂—CH₂—;
R³ denotes hydrogen, fluorine, OH or methoxy;
   optionally in the form of the pharmaceutically acceptable acid addition salts, hydrates or solvates thereof, at least one further active substance 2.

In another aspect the present invention relates to the above-mentioned new compounds of formula 1 in the form of the individual optical isomers, mixtures of the individual enantiomers or racemates. Particularly preferred are compounds of formula 1 in the form of the enantiomerically pure compounds, while the R-enantiomers of the compounds of formula 1 according to the invention are of exceptional importance. The R-enantiomers of the compounds of formula 1 can be represented by general formula R-1 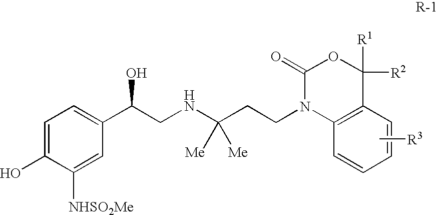
wherein the groups R¹, R² and R³ may have the meanings given above.

Methods of separating racemates into the respective enantiomers are known in the art and may be used analogously to prepare the enantiomerically pure R- and S-enantiomers of the compounds of formula 1.

Also particularly preferred are pharmaceutical combinations which contain, in addition to one or more, preferably one compound of general formula 1 selected from among the compounds
1.1: N-(5-{2-[1,1-dimethyl-3-(4-methyl-2-oxo-4H-benzo[d][1,3]oxazin-1-yl)-propylamino]-1-hydroxy-ethyl}-2-hydroxy-phenyl)-methanesulphonamide
1.2: N-(5-{2-[1,1-dimethyl-3-(2-oxo-4H-benzo[d][1,3]oxazin-1-yl)-propylamino]-1-hydroxy-ethyl}-2-hydroxy-phenyl)-methanesulphonamide
1.3: N-(5-{2-[3-(4-ethyl-2-oxo-4H-benzo[d][1,3]oxazin-1-yl)-1,1-dimethyl-propylamino]-1-hydroxy-ethyl}-2-hydroxy-phenyl)-methanesulphonamide
1.4: N-(5-{2-[3-(4,4-dimethyl-2-oxo-4H-benzo[d][1,3]oxazin-1-yl)-1,1-dimethyl-propylamino]-1-hydroxy-ethyl}-2-hydroxy-phenyl)-methanesulphonamide
1.5: N-(2-hydroxy-5-{1-hydroxy-2-[3-(6-hydroxy-4,4-dimethyl-2-oxo-4H-benzo[d][1,3]oxazin-1-yl)-1,1-dimethyl-propylamino]-ethyl}-phenyl)-methanesulphonamide
1.6: N-(2-hydroxy-5-{1-hydroxy-2-[3-(6-methoxy-4,4-dimethyl-2-oxo-4H-benzo[d][1,3]oxazin-1-yl)-1,1-dimethyl-propylamino]-ethyl}-phenyl)-methanesulphonamide
1.7: N-(5-{2-[1,1-dimethyl-3-(2-oxo-4,4-dipropyl-4H-benzo[d][1,3]oxazin-1-yl)-propylamino]-1-hydroxy-ethyl}-2-hydroxy-phenyl)-methanesulphonamide
1.8: N-[5-(2-{1,1-dimethyl-3-[spiro(cyclohexane-1,4′-2H-3′,1′-benzoxazin)-2′-oxo-1-yl]-propylamino}-1-hydroxy-ethyl)-2-hydroxy-phenyl]-methanesulphonamide
1.9: N-[5-(2-{1,1-dimethyl-3-[spiro(cyclopropyl-1,4′-2H-3′,1′-benzoxazin)-2′-oxo-1-yl]-propylamino}-1-hydroxy-ethyl)-2-hydroxy-phenyl]-methanesulphonamide
1.10: N-(5-{2-[3-(4,4-diethyl-2-oxo-4H-benzo[d][1,3]oxazin-1-yl)-1,1-dimethyl-propylamino]-1-hydroxy-ethyl}-2-hydroxy-phenyl)-methanesulphonamide:
1.11: N-(5-{2-[3-(4,4-diethyl-6-fluoro-2-oxo-4H-benzo[d][1,3]oxazin-1-yl)-1,1-dimethyl-propylamino]-1-hydroxy-ethyl}-2-hydroxy-phenyl)-methanesulphonamide
1.12: N-(5-{2-[3-(4,4-diethyl-7-fluoro-2-oxo-4H-benzo[d][1,3]oxazin-1-yl)-1,1-dimethyl-propylamino]-1-hydroxy-ethyl}-2-hydroxy-phenyl)-methanesulphonamide
1.13: N-(5-{2-[3-(4,4-diethyl-8-methoxy-2-oxo-4H-benzo[d][1,3]oxazin-1-yl)-1,1-dimethyl-propylamino]-1-hydroxy-ethyl}-2-hydroxy-phenyl)-methanesulphonamide
1.14: N-(5-{2-[3-(4,4-diethyl-6-methoxy-2-oxo-4H-benzo[d][1,3]oxazin-1-yl)-1,1-dimethyl-propylamino]-1-hydroxy-ethyl}-2-hydroxy-phenyl)-methanesulphonamide
   optionally in the form of the tautomers, mixtures of tautomers, hydrates or solvates thereof.

Also particularly preferred are pharmaceutical combinations which contain in addition to one or more, preferably one compound of general formula 1 selected from among the compounds
1.7: N-(5-{2-[1,1-dimethyl-3-(2-oxo-4,4-dipropyl-4H-benzo[d][1,3]oxazin-1-yl)-propylamino]-1-hydroxy-ethyl}-2-hydroxy-phenyl)-methanesulphonamide
1.8: N-[5-(2-{1,1-dimethyl-3-[spiro(cyclohexane-1,4′-2H-3′,1′-benzoxazin)-2′-oxo-1-yl]-propylamino}-1-hydroxy-ethyl)-2-hydroxy-phenyl]-methanesulphonamide
1.9: N-[5-(2-{1,1-dimethyl-3-[spiro(cyclopropyl-1,4′-2H-3′, 1′-benzoxazin)-2′-oxo-1-yl]-propylamino}-1-hydroxy-ethyl)-2-hydroxy-phenyl]-methanesulphonamide
1.10: N-(5-{2-[3-(4,4-diethyl-2-oxo-4H-benzo[d][1,3]oxazin-1-yl)-1,1-dimethyl-propylamino]-1-hydroxy-ethyl}-2-hydroxy-phenyl)-methanesulphonamide:
1.11: N-(5-{2-[3-(4,4-diethyl-6-fluoro-2-oxo-4H-benzo[d][1,3]oxazin-1-yl)-1,1-dimethyl-propylamino]-1-hydroxy-ethyl}-2-hydroxy-phenyl)-methanesulphonamide
1.12: N-(5-{2-[3-(4,4-diethyl-7-fluoro-2-oxo-4H-benzo[d][1,3]oxazin-1-yl)-1,1-dimethyl-propylamino]-1-hydroxy-ethyl}-2-hydroxy-phenyl)-methanesulphonamide
1.13: N-(5-{2-[3-(4,4-diethyl-8-methoxy-2-oxo-4H-benzo[d][1,3]oxazin-1-yl)-1,1-dimethyl-propylamino]-1-hydroxy-ethyl}-2-hydroxy-phenyl)-methanesulphonamide
1.14: N-(5-{2-[3-(4,4-diethyl-6-methoxy-2-oxo-4H-benzo[d][1,3]oxazin-1-yl)-1,1-dimethyl-propylamino]-1-hydroxy-ethyl}-2-hydroxy-phenyl)-methanesulphonamide
   optionally in the form of the tautomers, mixtures of tautomers, hydrates or solvates thereof.

Preferred pharmaceutical combinations contain in addition to one or more, preferably one compound of formula 1, as a further active substance, one or more, preferably one anticholinergic 2a, optionally combined with pharmaceutically acceptable excipients.

In the pharmaceutical combinations according to the invention the anticholinergic 2a is preferably selected from among the tiotropium salts (2a.1), oxitropium salts (2a.2), flutropium salts (2a.3), ipratropium salts (2a.4), glycopyrronium salts (2a.5), trospium salts (2a.6) and the compounds of formulae 2a.7 to 2a.13.

In the above-mentioned salts 2a.1 to 2a.6 the cations tiotropium, oxitropium, flutropium, ipratropium, glycopyrronium and trospium are the pharmacologically active ingredients. Explicit reference to the above-mentioned cations is indicated by the terminology 2a.1′ to 2a.6′. Any reference to the above-mentioned salts 2a.1 to 2a.6 naturally also includes a reference to the corresponding cations tiotropium (2a.1′), oxitropium (2a.2′), flutropium (2a.3′), ipratropium (2a.4′), glycopyrronium (2a.5′), trospium (2a.6′).

By the salts 2a.1 to 2a.6 are meant according to the invention those compounds which contain in addition to the cations tiotropium (2a.1′), oxitropium (2a.2′), flutropium (2a.3′), ipratropium (2a.4′), glycopyrronium (2a.5′) and trospium (2a.6′) as counter-ion (anion) chloride, bromide, iodide, sulphate, phosphate, methanesulphonate, nitrate, maleate, acetate, citrate, fumarate, tartrate, oxalate, succinate, benzoate or p-toluenesulphonate, while chloride, bromide, iodide, sulphate, methanesulphonate or p-toluenesulphonate are preferred as counter-ions. Of all the salts the chlorides, bromides, iodides and methanesulphonates are particularly preferred. In the case of the trospium salts (2a.6) the chloride is particularly preferred. In the case of the other salts 2a.1 to 2a.5 the methanesulphonates and bromides are of particular importance. Of particular importance are medicament combinations which contain tiotropium salts (2a.1), oxitropium salts (2a.2) or ipratropium salts (2a.4), while the respective bromides are of particular importance according to the invention. Tiotropium bromide is of particular importance. The above-mentioned salts may optionally be present in the medicament combinations according to the invention in the form of the solvates or hydrates thereof, preferably in the form of their hydrates. In the case of tiotropium bromide the medicament combinations according to the invention preferably contain it in the form of the crystalline tiotropium bromide monohydrate which is known from WO 02/30928. If tiotropium bromide is used in anhydrous form in the medicament combinations according to the invention, preferably anhydrous crystalline tiotropium bromide is used, which is known from WO 03/000265.

Examples of novel preferred medicament combinations of preferred compounds of formula 1 with the above-mentioned anticholinergics 2a.1 to 2a.6 are combinations containing the compounds 1.4 and 2a.1; 1.4 and 2a.2; 1.4 and 2a.3; 1.4 and 2a.4; 1.4 and 2a.5; 1.4 and 2a.6; 1.5 and 2a.1; 1.5 and 2a.2; 1.5 and 2a.3; 1.5 and 2a.4; 1.5 and 2a.5; 1.5 and 2a.6; 1.6 and 2a.1; 1.6 and 2a.2; 1.6 and 2a.3; 1.6 and 2a.4; 1.6 and 2a.5; 1.6 and 2a.6; 1.7 and 2a.1; 1.7 and 2a.2; 1.7 and 2a.3; 1.7 and 2a.4; 1.7 and 2a.5; 1.7 and 2a.6; 1.8 and 2a.1; 1.8 and 2a.2; 1.8 and 2a.3; 1.8 and 2a.4; 1.8 and 2a.5; 1.8 and 2a.6; 1.9 and 2a.1; 1.9 and 2a.2; 1.9 and 2a.3; 1.9 and 2a.4; 1.9 and 2a.5; 1.9 and 2a.6; 1.10 and 2a.1; 1.10 and 2a.2; 1.10 and 2a.3; 1.10 and 2a.4; 1.10 and 2a.5; 1.10 and 2a.6; 1.11 and 2a.1; 1.11 and 2a.2; 1.11 and 2a.3; 1.11 and 2a.4; 1.11 and 2a.5; 1.11 and 2a.6; 1.12 and 2a.1; 1.12 and 2a.2; 1.12 and 2a.3; 1.12 and 2a.4; 1.12 and 2a.5; 1.12 and 2a.6; 1.13 and 2a.1; 1.13 and 2a.2; 1.13 and 2a.3; 1.13 and 2a.4; 1.13 and 2a.5; 1.13 and 2a.6; 1.14 and 2a.1; 1.14 and 2a.2; 1.14 and 2a.3; 1.14 and 2a.4; 1.14 and 2a.5; 1.14 and 2a.6; in each case optionally in the form of the racemates, enantiomers or diastereomers thereof and optionally in the form of the pharmacologically acceptable acid addition salts, solvates and/or hydrates thereof.

Of the combinations mentioned above, the preferred ones according to the invention are those which contain as compound of formula 1 one of the compounds 1.7, 1.8, 1.9, 1.10, 1.11, 1.12, 1.13, or 1.14. Of the above-mentioned combinations the preferred ones according to the invention are also those which contain as compound 2a one of the compounds 2a.1, 2a.2 or 2a.4, while those combinations which contain the compound 2a.1 are of particular importance according to the invention.

Optionally the above-mentioned anticholinergics have chiral carbon centres. In this case the medicament combinations according to the invention may contain the anticholinergics in the form of their enantiomers, mixtures of enantiomers or racemates, while enantiomerically pure anticholinergics are preferably used.

In another preferred embodiment of the present invention the anticholinergics 2a contained in the medicament combinations according to the invention are selected from the salts of formula 2a.7 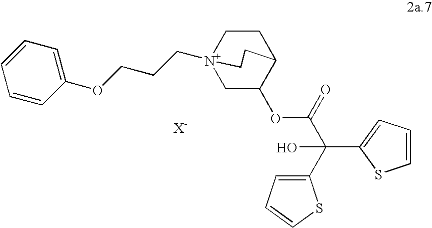
wherein
X⁻ denotes an anion with a single negative charge, preferably an anion selected from among the fluoride, chloride, bromide, iodide, sulphate, phosphate, methanesulphonate, nitrate, maleate, acetate, citrate, fumarate, tartrate, oxalate, succinate, benzoate and p-toluenesulphonate,
   optionally in the form of the racemates, enantiomers or hydrates thereof. Preferred medicament combinations contain salts of formula 2a.7, wherein
X⁻ denotes an anion with a single negative charge, preferably an anion selected from among the fluoride, chloride, bromide, methanesulphonate and p-toluenesulphonate, preferably bromide,
   optionally in the form of the racemates, enantiomers or hydrates thereof. Preferred medicament combinations contain salts of formula 2a.7, wherein
X⁻ denotes an anion with a single negative charge, preferably an anion selected from among the chloride, bromide and methanesulphonate, preferably bromide,
   optionally in the form of the racemates, enantiomers or hydrates thereof.

Particularly preferred medicament combinations contain the compound of formula 2a.7 in the form of the bromide. Of particular importance are those medicament combinations which contain the enantiomers of formula 2a.7-ene 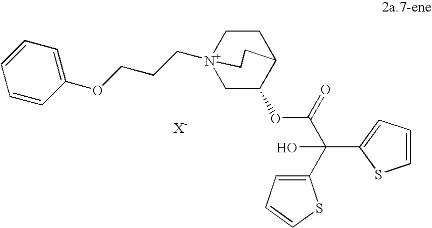
wherein X⁻ may have the meanings given above.

Examples of novel medicament combinations of preferred compounds of formula 1 with the above-mentioned anticholinergics 2a.7 are combinations containing the compounds 1.4 and 2a.7; 1.4 and 2a.7-ene; 1.5 and 2a.7; 1.5 and 2a.7-ene; 1.6 and 2a.7; 1.6 and 2a.7-ene; 1.7 and 2a.7; 1.7 and 2a.7-ene; 1.8 and 2a.7; 1.8 and 2a.7-ene; 1.9 and 2a.7; 1.9 and 2a.7-ene; 1.10 and 2a.7; 1.10 and 2a.7-ene; 1.11 and 2a.7; 1.11 and 2a.7-ene; 1.12 and 2a.7; 1.12 and 2a.7-ene; 1.13 and 2a.7; 1.13 and 2a.7-ene; 1.14 and 2a.7 or 1.14 and 2a.7-ene; in each case optionally in the form of the racemates, enantiomers or diastereomers thereof and optionally in the form of the pharmacologically acceptable acid addition salts, solvates and/or hydrates thereof.

Of the above-mentioned combinations the preferred ones according to the invention are those which contain as the compound of formula 1 one of the compounds 1.7, 1.8, 1.9, 1.10, 1.11, 1.12, 1.13, or 1.14.

In another preferred embodiment of the present invention the anticholinergics 2a contained in the pharmaceutical combinations according to the invention are selected from among the salts of formula 2a.8 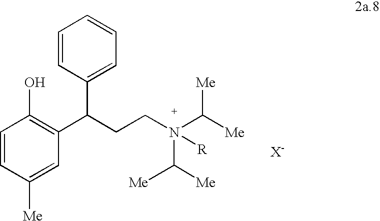
wherein R denotes either methyl (2a.8.1) or ethyl (2a.8.2) and wherein X⁻ may have the meanings given above. In an alternative embodiment the compound of formula 2a.8 is present in the form of the free base 2a.8-base

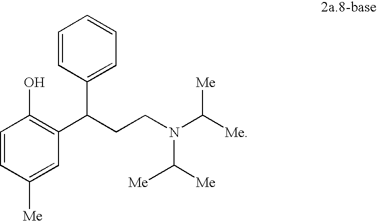

The pharmaceutical combinations according to the invention may contain the anticholinergic of formula 2a.8 (or 2a.8-base) in the form of the enantiomers, mixtures of enantiomers or racemates thereof. Preferably the anticholinergics of formula 2a.8 (or 2a.8-base) are present in the form of the R-enantiomers thereof.

Examples of novel pharmaceutical combinations of preferred compounds of formula 1 with the above-mentioned anticholinergics 2a.8 are combinations containing the compounds 1.4 and 2a.8.1; 1.4 and 2a.8.2; 1.5 and 2a.8.1; 1.5 and 2a.8.2; 1.6 and 2a.8.1; 1.6 and 2a.8.2; 1.7 and 2a.8.1; 1.7 and 2a.8.2; 1.8 and 2a.8.1; 1.8 and 2a.8.2; 1.9 and 2a.8.1; 1.9 and 2a.8.2; 1.10 and 2a.8.1; 1.10 and 2a.8.2; 1.11 and 2a.8.1; 1.11 and 2a.8.2; 1.12 and 2a.8.1; 1.12 and 2a.8.2; 1.13 and 2a.8.1; 1.13 and 2a.8.2; 1.14 and 2a.8.1 or 1.14 and 2a.8.2; in each case optionally in the form of the racemates, enantiomers or diastereomers thereof and optionally in the form of the pharmacologically acceptable acid addition salts, solvates and/or hydrates thereof.

Of the above-mentioned combinations the preferred ones according to the invention are those that contain as compound of formula 1 one of the compounds 1.7, 1.8, 1.9, 1.10, 1.11, 1.12, 1.13, or 1.14.

In another preferred embodiment of the present invention the anticholinergics contained in the pharmaceutical combinations according to the invention 2a are selected from among the compounds of formula 2a.9 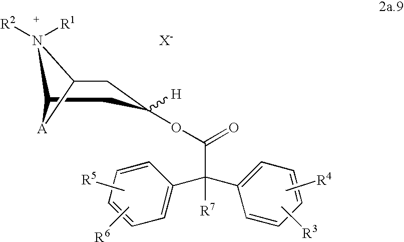
wherein
A denotes a double-bonded group selected from the groups 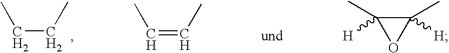
X⁻ one of the above-mentioned anions with a single negative charge, preferably chloride, bromide or methanesulphonate,
R¹ and R² which may be identical or different denote a group selected from among methyl, ethyl, n-propyl and iso-propyl, which may optionally be substituted by hydroxy or fluorine, preferably unsubstituted methyl;
R³, R⁴, R⁵ and R⁶, which may be identical or different, denote hydrogen, methyl, ethyl, methyloxy, ethyloxy, hydroxy, fluorine, chlorine, bromine, CN, CF₃ or NO₂;
R⁷ denotes hydrogen, methyl, ethyl, methyloxy, ethyloxy, —CH₂—F, —CH₂—CH₂—F, —O—CH₂F, —O—CH₂CH₂F, —CH₂OH, —CH₂CH₂OH, CF₃, —CH₂—OMe, —CH₂—CH₂—OMe, —CH₂—OEt, —CH₂—CH₂—OEt, —O—COMe, —O—COEt, —O—COCF₃, fluorine, chlorine or bromine.

The compounds of formula 2a.9 are known in the art (WO 02/32899).

Within the scope of the pharmaceutical combinations according to the invention preferred compounds of formula 2a.9 are those wherein
X⁻ denotes bromide;
R¹ and R² which may be identical or different, denote methyl or ethyl, preferably methyl;
R³, R⁴, R⁵ and R⁶, which may be identical or different, denote hydrogen, methyl, methyloxy, chlorine or fluorine;
R⁷ denotes hydrogen, methyl or fluorine.

Of particular importance are pharmaceutical combinations which contain compounds of formula 2a.9, wherein
A denotes a double-bonded group selected from

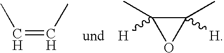

Of particular importance are those medicament combinations which contain in addition to a compound of formula 1 one of the following compounds of formula 2a.9:
tropenol 2,2-diphenylpropionate-methobromide (2a.9.1),
scopine 2,2-diphenylpropionate-methobromide (2a.9.2),
scopine 2-fluoro-2,2-diphenylacetate-methobromide (2a.9.3),
tropenol 2-fluoro-2,2-diphenylacetate-methobromide (2a.9.4),

The compounds of formula 2a.9 may optionally be present in the form of their enantiomers, mixtures of their enantiomers or racemates, as well as optionally in the form of the hydrates and/or solvates thereof.

Examples of novel pharmaceutical combinations of preferred compounds of formula 1 with the above-mentioned anticholinergics 2a.9 are combinations containing the compounds 1.4 and 2a.9.1; 1.4 and 2a.9.2; 1.4 and 2a.9.3; 1.4 and 2a.9.4; 1.5 and 2a.9.1; 1.5 and 2a.9.2; 1.5 and 2a.9.3; 1.5 and 2a.9.4; 1.6 and 2a.9.1; 1.6 and 2a.9.2; 1.6 and 2a.9.3; 1.6 and 2a.9.4; 1.7 and 2a.9.1; 1.7 and 2a.9.2; 1.7 and 2a.9.3; 1.7 and 2a.9.4; 1.8 and 2a.9.1; 1.8 and 2a.9.2; 1.8 and 2a.9.3; 1.8 and 2a.9.4; 1.9 and 2a.9.1; 1.9 and 2a.9.2; 1.9 and 2a.9.3; 1.9 and 2a.9.4; 1.10 and 2a.9.1; 1.10 and 2a.9.2; 1.10 and 2a.9.3; 1.10 and 2a.9.4; 1.11 and 2a.9.1; 1.11 and 2a.9.2; 1.11 and 2a.9.3; 1.11 and 2a.9.4; 1.12 and 2a.9.1; 1.12 and 2a.9.2; 1.12 and 2a.9.3; 1.12 and 2a.9.4; 1.13 and 2a.9.1; 1.13 and 2a.9.2; 1.13 and 2a.9.3; 1.13 and 2a.9.4; 1.14 and 2a.9.1; 1.14 and 2a.9.2; 1.14 and 2a.9.3; 1.14 and 2a.9.4; in each case optionally in the form of the racemates, enantiomers or diastereomers thereof and optionally in the form of the pharmacologically acceptable acid addition salts, solvates and/or hydrates thereof.

Of the above-mentioned combinations the preferred ones according to the invention are those that contain as compound of formula 1 one of the compounds 1.7, 1.8, 1.9, 1.10, 1.11, 1.12, 1.13, or 1.14. Of the above-mentioned combinations the preferred ones according to the invention are also those which contain as compound 2a.9 one of the compounds 2a.9.1 or 2a.9.2, while those combinations that contain the compound 2a.9.2 are of particular importance according to the invention.

In another preferred embodiment of the present invention the anticholinergics 2a contained in the pharmaceutical combinations according to the invention are selected from among the compounds of formula 2a.10 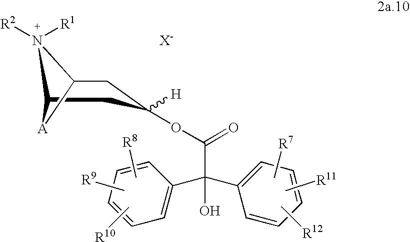
wherein
A, X⁻ R¹ and R² may have the meanings given above and wherein
R⁷, R⁸, R⁹, R¹⁰, R¹¹ and R¹², which may be identical or different, denote hydrogen, methyl, ethyl, methyloxy, ethyloxy, hydroxy, fluorine, chlorine, bromine, CN, CF₃ or NO₂, while at least one of the groups R⁷, R⁸, R⁹, R¹⁰, R¹¹ and R¹² may not be hydrogen.

The compounds of formula 2a.10 are known in the art (WO 02/32898).

Within the scope of the pharmaceutical combinations according to the invention preferred compounds of formula 2a.10 are those wherein
A denotes a double-bonded group selected from 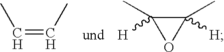
X⁻ denotes bromide;
R¹ and R² which may be identical or different, denote methyl or ethyl, preferably methyl;
R⁷, R⁸, R⁹, R¹⁰, R¹¹ and R¹², which may be identical or different, denote hydrogen, fluorine, chlorine or bromine, preferably fluorine, while at least one of the groups R⁷, R⁸, R⁹, R¹⁰, R¹¹ and R¹² may not be hydrogen.

Of particular importance are those medicament combinations which contain, in addition to a compound of formula 1, one of the following compounds of formula 2a.10:
tropenol 3,3′,4,4′-tetrafluorobenzilate-methobromide (2a.10.1),
scopine 3,3′,4,4′-tetrafluorobenzilate-methobromide (2a.10.2),
tropenol 4,4′-difluorobenzilate-methobromide (2a.10.3),
scopine 4,4′-difluorobenzilate-methobromide (2a.10.4),
tropenol 3,3′-difluorobenzilate-methobromide (2a.10.5),
scopine 3,3′-difluorobenzilate-methobromide (2a.10.6).

The compounds of formula 2a.10 may optionally be present in the form of the enantiomers, mixtures of enantiomers or racemates thereof, as well as optionally in the form of the hydrates and/or solvates thereof.

Examples of novel pharmaceutical combinations of preferred compounds of formula 1 with the above-mentioned anticholinergics 2a.10 are combinations containing the compounds 1.4 and 2a.10.1; 1.4 and 2a.10.2; 1.4 and 2a.10.3; 1.4 and 2a.10.4; 1.4 and 2a.10.5; 1.4 and 2a.10.6; 1.5 and 2a.10.1; 1.5 and 2a.10.2; 1.5 and 2a.10.3; 1.5 and 2a.10.4; 1.5 and 2a.10.5; 1.5 and 2a.10.6; 1.6 and 2a.10.1; 1.6 and 2a.10.2; 1.6 and 2a.10.3; 1.6 and 2a.10.4; 1.6 and 2a.10.5; 1.6 and 2a.10.6; 1.7 and 2a.10.1; 1.7 and 2a.10.2; 1.7 and 2a.10.3; 1.7 and 2a.10.4; 1.7 and 2a.10.5; 1.7 and 2a.10.6; 1.8 and 2a.10.1; 1.8 and 2a.10.2; 1.8 and 2a.10.3; 1.8 and 2a.10.4; 1.8 and 2a.10.5; 1.8 and 2a.10.6; 1.9 and 2a.10.1; 1.9 and 2a.10.2; 1.9 and 2a.10.3; 1.9 and 2a.10.4; 1.9 and 2a.10.5; 1.9 and 2a.10.6; 1.10 and 2a.10.1; 1.10 and 2a.10.2; 1.10 and 2a.10.3; 1.10 and 2a.10.4; 1.10 and 2a.10.5; 1.10 and 2a.10.6; 1.11 and 2a.10.1; 1.11 and 2a.10.2; 1.11 and 2a.10.3; 1.11 and 2a.10.4; 1.11 and 2a.10.5; 1.11 and 2a.10.6; 1.12 and 2a.10.1; 1.12 and 2a.10.2; 1.12 and 2a.10.3; 1.12 and 2a.10.4; 1.12 and 2a.10.5; 1.12 and 2a.10.6; 1.13 and 2a.10.1; 1.13 and 2a.10.2; 1.13 and 2a.10.3; 1.13 and 2a.10.4; 1.13 and 2a.10.5; 1.13 and 2a.10.6; 1.14 and 2a.10.1; 1.14 and 2a.10.2; 1.14 and 2a.10.3; 1.14 and 2a.10.4; 1.14 and 2a.10.5; 1.14 and 2a.10.6; in each case optionally in the form of the racemates, enantiomers or diastereomers thereof and optionally in the form of the pharmacologically acceptable acid addition salts, solvates and/or hydrates thereof.

Of the above-mentioned combinations the preferred ones according to the invention are those that contain as compound of formula 1 one of the compounds 1.7, 1.8, 1.9, 1.10, 1.11, 1.12, 1.13, or 1.14. Of the above-mentioned combinations the preferred ones according to the invention are also those which contain as compound 2a.10 one of the compounds 2a.10.1, 2a.10.2, 2a.10.3 or 2a.10.4, while those combinations which contain the compounds 2a.10.1 or 2a.10.2 are of particular importance according to the invention.

In another preferred embodiment of the present invention the anticholinergics 2a contained in the pharmaceutical combinations according to the invention are selected from among the compounds of formula 2a.11 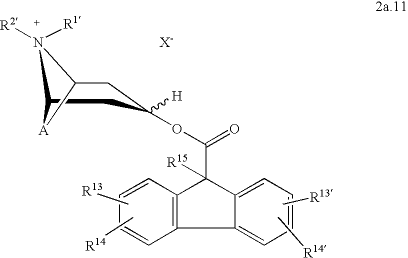
wherein
A and X⁻ may have the meanings given above and wherein
R¹⁵ denotes hydrogen, hydroxy, methyl, ethyl, —CF₃, CHF₂ or fluorine;
R^{1′} and R^{2′} which may be identical or different, denote C₁-C₅-alkyl, which may optionally be substituted by C₃-C₆-cycloalkyl, hydroxy or halogen, or
   R^{1′} and R^{2′} together denote a —C₃-C₅-alkylene bridge;
R¹³, R¹⁴, R^{13′} and R^{14′}, which may be identical or different, denote hydrogen, —C₁-C₄-alkyl, —C₁-C₄-alkyloxy, hydroxy, —CF₃, —CHF₂, CN, NO₂ or halogen.

The compounds of formula 2a.11 are known in the art (WO 03/064419).

Within the scope of the pharmaceutical combinations according to the invention preferred compounds of formula 2a.11 are those wherein
A denotes a double-bonded group selected from 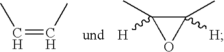
X⁻ denotes an anion selected from chloride, bromide and methanesulphonate, preferably bromide;
R¹⁵ denotes hydroxy, methyl or fluorine, preferably methyl or hydroxy;
R^{1′} and R^{2′} which may be identical or different, denote methyl or ethyl, preferably methyl;
R¹³, R¹⁴, R^{13′} and R^{14′} which may be identical or different, denote hydrogen, —CF₃, —CHF₂ or fluorine, preferably hydrogen or fluorine.

Within the scope of the pharmaceutical combinations according to the invention particularly preferred compounds of formula 2a.11 are those wherein
A denotes a double-bonded group selected from 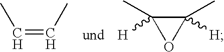
X⁻ denotes bromide;
R¹⁵ denotes hydroxy or methyl, preferably methyl;
R^{1′} and R^{2′} which may be identical or different, denote methyl or ethyl, preferably methyl;
R¹³, R¹⁴, R^{13′} and R^{14′} which may be identical or different, denote hydrogen or fluorine.

Of particular importance are those medicament combinations which contain, in addition to a compound of formula 1, one of the following compounds of formula 2a.11:
tropenol 9-hydroxy-fluorene-9-carboxylate methobromide (2a.11.1);
tropenol 9-fluoro-fluorene-9-carboxylate methobromide (2a.11.2);
scopine 9-hydroxy-fluorene-9-carboxylate methobromide (2a.11.3);
scopine 9-fluoro-fluorene-9-carboxylate methobromide (2a.11.4);
tropenol 9-methyl-fluorene-9-carboxylate methobromide (2a.11.5);
scopine 9-methyl-fluorene-9-carboxylate methobromide (2a.11.6);

The compounds of formula 2a.11 may optionally be present in the form of the enantiomers, mixtures of enantiomers or racemates thereof, as well as optionally in the form of the hydrates and/or solvates thereof.

Examples of novel pharmaceutical combinations of preferred compounds of formula 1 with the above-mentioned anticholinergics 2a.11 are combinations containing the compounds 1.4 and 2a.11.1; 1.4 and 2a.11.2; 1.4 and 2a.11.3; 1.4 and 2a.11.4; 1.4 and 2a.11.5; 1.4 and 2a.11.6; 1.5 and 2a.11.1; 1.5 and 2a.11.2; 1.5 and 2a.11.3; 1.5 and 2a.11.4; 1.5 and 2a.11.5; 1.5 and 2a.11.6; 1.6 and 2a.11.1; 1.6 and 2a.11.2; 1.6 and 2a.11.3; 1.6 and 2a.11.4; 1.6 and 2a.11.5; 1.6 and 2a.11.6; 1.7 and 2a.11.1; 1.7 and 2a.11.2; 1.7 and 2a.11.3; 1.7 and 2a.11.4; 1.7 and 2a.11.5; 1.7 and 2a.11.6; 1.8 and 2a.11.1; 1.8 and 2a.11.2; 1.8 and 2a.11.3; 1.8 and 2a.11.4; 1.8 and 2a.11.5; 1.8 and 2a.11.6; 1.9 and 2a.11.1; 1.9 and 2a.11.2; 1.9 and 2a.11.3; 1.9 and 2a.11.4; 1.9 and 2a.11.5; 1.9 and 2a.11.6; 1.10 and 2a.11.1; 1.10 and 2a.11.2; 1.10 and 2a.11.3; 1.10 and 2a.11.4; 1.10 and 2a.11.5; 1.10 and 2a.11.6; 1.11 and 2a.11.1; 1.11 and 2a.11.2; 1.11 and 2a.11.3; 1.11 and 2a.11.4; 1.11 and 2a.11.5; 1.11 and 2a.11.6; 1.12 and 2a.11.1; 1.12 and 2a.11.2; 1.12 and 2a.11.3; 1.12 and 2a.11.4; 1.12 and 2a.11.5; 1.12 and 2a.11.6; 1.13 and 2a.11.1; 1.13 and 2a.11.2; 1.13 and 2a.11.3; 1.13 and 2a.11.4; 1.13 and 2a.11.5; 1.13 and 2a.11.6; 1.14 and 2a.11.1; 1.14 and 2a.11.2; 1.14 and 2a.11.3; 1.14 and 2a.11.4; 1.14 and 2a.11.5; 1.14 and 2a.11.6; in each case optionally in the form of the racemates, enantiomers or diastereomers thereof and optionally in the form of the pharmacologically acceptable acid addition salts, solvates and/or hydrates thereof.

Of the above-mentioned combinations the preferred ones according to the invention are those that contain as compound of formula 1 one of the compounds 1.7, 1.8, 1.9, 1.10, 1.11, 1.12, 1.13, or 1.14. Of the above-mentioned combinations the preferred ones according to the invention are also those which contain as compound 2a.11 one of the compounds 2a.11.2, 2a.11.4, 2a.11.5 or 2a.11.6, while those combinations which contain the compounds 2a.11.5 or 2a.11.6 are of particular importance according to the invention.

In another preferred embodiment of the present invention the anticholinergics 2a contained in the pharmaceutical combinations according to the invention are selected from among the compounds of formula 2a.12 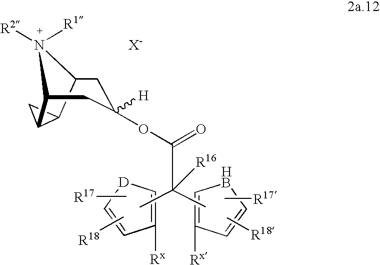
wherein X⁻ may have the meanings given above and wherein
D and B which may be identical or different, preferably identical, denote O, S, NH, CH₂, CH═CH or N(C₁-C₄-alkyl);
R¹⁶ denotes hydrogen, hydroxy, —C₁-C₄-alkyl, —C₁-C₄-alkyloxy, —C₁-C₄-alkylene-halogen, —O—C₁-C₄-alkylene-halogen, —C₁-C₄-alkylene-OH, —CF₃, CHF₂, —C₁-C₄-alkylene-C₁-C₄-alkyloxy, —O—COC₁-C₄-alkyl, —O—COC₁-C₄-alkylene-halogen, —C₁-C₄-alkylene-C₃-C₆-cycloalkyl, —O—COCF₃ or halogen;
R^{1″} and R^{2″} which may be identical or different, denote —C₁-C₅-alkyl, which may optionally be substituted by —C₃-C₆-cycloalkyl, hydroxy or halogen, or R^{1″} and R^{2″} together denote a —C₃-C₅-alkylene bridge;
R¹⁷, R¹⁸, R^{17′} and R^{18′}, which may be identical or different, denote hydrogen, —C₁-C₄-alkyl, —C₁-C₄-alkyloxy, hydroxy, —CF₃, —CHF₂, CN, NO₂ or halogen;
R^{x} and R^{x′} which may be identical or different, denote hydrogen, —C₁-C₄-alkyl, —C₁-C₄-alkyloxy, hydroxy, —CF₃, —CHF₂, CN, NO₂ or halogen, or Rx and Rx together denote a single bond or one of the double-bonded groups O, S, NH, CH₂, CH₂—CH₂, N(C₁-C₄-alkyl), CH(C₁-C₄-alkyl) and —C(C₁-C₄-alkyl)₂.

The compounds of formula 2a.12 are known in the art (WO 03/064418).

Within the scope of the pharmaceutical combinations according to the invention preferred compounds of formula 2a.12 are those wherein
X⁻ denotes chloride, bromide or methanesulphonate, preferably bromide;
D and B which may be identical or different, preferably identical, denote O, S, NH or CH═CH;
R¹⁶ denotes hydrogen, hydroxy, —C₁-C₄-alkyl, —C₁-C₄-alkyloxy, —CF₃, —CHF₂, fluorine, chlorine or bromine;
R^{1″} and R^{2″} which may be identical or different, denote C₁-C₄-alkyl, which may optionally be substituted by hydroxy, fluorine, chlorine or bromine, or R^{1″} and R^{2″} together denote a —C₃-C₄-alkylene bridge;
R¹⁷, R¹⁸, R^{17′} and R^{18′}, which may be identical or different, denote hydrogen, C₁-C₄-alkyl, C₁-C₄-alkyloxy, hydroxy, —CF₃, —CHF₂, CN, NO₂, fluorine, chlorine or bromine;
R^{x} and R^{x′} which may be identical or different, denote hydrogen, C₁-C₄-alkyl, C₁-C₄-alkyloxy, hydroxy, —CF₃, —CHF₂, CN, NO₂, fluorine, chlorine or bromine, or R^{x} and R^{x′} together denote a single bond or a double-bonded group selected from O, S, NH— and CH₂.

Within the scope of the pharmaceutical combinations according to the invention particularly preferred compounds of formula 2a.12 are those wherein
X⁻ denotes chloride, bromide, or methanesulphonate, preferably bromide;
D and B which may be identical or different, preferably identical, denote S or CH═CH;
R¹⁶ denotes hydrogen, hydroxy or methyl;
R^{1″} and R^{2″} which may be identical or different, denote methyl or ethyl;
R¹⁷, R¹⁸, R^{17′} and R^{18′}, which may be identical or different, denote hydrogen, —CF₃ or fluorine, preferably hydrogen;
R^{x} and R^{x′} which may be identical or different, denote hydrogen, —CF₃ or fluorine, preferably hydrogen, or R^{x} and R^{x′} together denote a single bond or —O.

Within the scope of the pharmaceutical combinations according to the invention other particularly preferred compounds of formula 2a.12 are those wherein
X⁻ denotes bromide;
D and B denote —CH═CH—;
R¹⁶ denotes hydrogen, hydroxy or methyl;
R^{1″} and R^{2″} denote methyl;
R¹⁷, R¹⁸, R^{17′} and R^{18′}, which may be identical or different, denote hydrogen or fluorine, preferably hydrogen;
R^{x} and R^{x′} which may be identical or different, denote hydrogen or fluorine, preferably hydrogen, or R^{x} and R^{x′} together represent a single bond or the group —O.

Of particular importance are those medicament combinations which contain in addition to a compound of formula 1 one of the following compounds of formula 2a.12:
cyclopropyltropine benzilate-methobromide (2a.12.1);
cyclopropyltropine 2,2-diphenylpropionate-methobromide (2a.12.2);
cyclopropyltropine 9-hydroxy-xanthene-9-carboxylate-methobromide (2a.12.3);
cyclopropyltropine 9-methyl-fluorene-9-carboxylate-methobromide (2a.12.4);
cyclopropyltropine 9-methyl-xanthene-9-carboxylate-methobromide (2a.12.5);
cyclopropyltropine 9-hydroxy-fluorene-9-carboxylate-methobromide (2a.12.6);
methyl cyclopropyltropine 4,4′-difluorobenzilate-methobromide (2a.12.7).

The compounds of formula 2a.12 may optionally be present in the form of the enantiomers, mixtures of enantiomers or racemates thereof, as well as optionally in the form of the hydrates and/or solvates thereof.

Examples of novel pharmaceutical combinations of preferred compounds of formula 1 with the above-mentioned anticholinergics 2a.12 are combinations containing the compounds 1.4 and 2a.12.1; 1.4 and 2a.12.2; 1.4 and 2a.12.3; 1.4 and 2a.12.4; 1.4 and 2a.12.5; 1.4 and 2a.12.6; 1.4 and 2a.12.7; 1.5 and 2a.12.1; 1.5 and 2a.12.2; 1.5 and 2a.12.3; 1.5 and 2a.12.4; 1.5 and 2a.12.5; 1.5 and 2a.12.6; 1.5 and 2a.12.7; 1.6 and 2a.12.1; 1.6 and 2a.12.2; 1.6 and 2a.12.3; 1.6 and 2a.12.4; 1.6 and 2a.12.5; 1.6 and 2a.12.6; 1.6 and 2a.12.7; 1.7 and 2a.12.1; 1.7 and 2a.12.2; 1.7 and 2a.12.3; 1.7 and 2a.12.4; 1.7 and 2a.12.5; 1.7 and 2a.12.6; 1.7 and 2a.12.7; 1.8 and 2a.12.1; 1.8 and 2a.12.2; 1.8 and 2a.12.3; 1.8 and 2a.12.4; 1.8 and 2a.12.5; 1.8 and 2a.12.6; 1.8 and 2a.12.7; 1.9 and 2a.12.1; 1.9 and 2a.12.2; 1.9 and 2a.12.3; 1.9 and 2a.12.4; 1.9 and 2a.12.5; 1.9 and 2a.12.6; 1.9 and 2a.12.7; 1.10 and 2a.12.1; 1.10 and 2a.12.2; 1.10 and 2a.12.3; 1.10 and 2a.12.4; 1.10 and 2a.12.5; 1.10 and 2a.12.6; 1.10 and 2a.12.7; 1.11 and 2a.12.1; 1.11 and 2a.12.2; 1.11 and 2a.12.3; 1.11 and 2a.12.4; 1.11 and 2a.12.5; 1.11 and 2a.12.6; 1.11 and 2a.12.7; 1.12 and 2a.12.1; 1.12 and 2a.12.2; 1.12 and 2a.12.3; 1.12 and 2a.12.4; 1.12 and 2a.12.5; 1.12 and 2a.12.6; 1.12 and 2a.12.7; 1.13 and 2a.12.1; 1.13 and 2a.12.2; 1.13 and 2a.12.3; 1.13 and 2a.12.4; 1.13 and 2a.12.5; 1.13 and 2a.12.6; 1.13 and 2a.12.7; 1.14 and 2a.12.1; 1.14 and 2a.12.2; 1.14 and 2a.12.3; 1.14 and 2a.12.4; 1.14 and 2a.12.5; 1.14 and 2a.12.6; 1.14 and 2a.12.7; in each case optionally in the form of the racemates, enantiomers or diastereomers thereof and optionally in the form of the pharmacologically acceptable acid addition salts, solvates and/or hydrates thereof.

Of the above-mentioned combinations the preferred ones according to the invention are those that contain as compound of formula 1 one of the compounds 1.7, 1.8, 1.9, 1.10, 1.11, 1.12, 1.13, or 1.14 contain. Of the above-mentioned combinations the preferred ones according to the invention are also those which contain as compound 2a.12 one of the compounds 2a.12.1, 2a.12.2, 2a.12.5 or 2a.12.7, while those combinations which contain the compounds 2a.12.1 or 2a.12.2 are of particular importance according to the invention.

In another preferred embodiment of the present invention the anticholinergics 2a contained in the pharmaceutical combinations according to the invention are selected from among the compounds of formula 2a.13 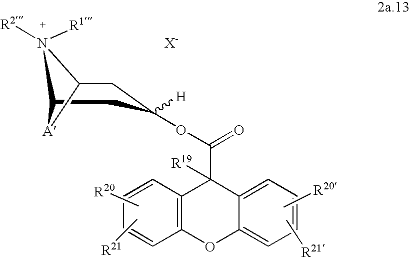
wherein X⁻ may have the meanings given above and wherein
A′ denotes a double-bonded group selected from 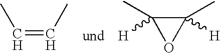
R¹⁹ denotes hydroxy, methyl, hydroxymethyl, ethyl, —CF₃, CHF₂ or fluorine;
R^{1′″} and R^{2′″} which may be identical or different, denote C₁-C₅-alkyl, which may optionally be substituted by C₃-C₆-cycloalkyl, hydroxy or halogen, or
   R^{1′″} and R^{2′″} together denote a —C₃-C₅-alkylene bridge;
R²⁰, R²¹, R^{20′} and R^{21′} which may be identical or different, denote hydrogen, —C₁-C₄-alkyl, —C₁-C₄-alkyloxy, hydroxy, —CF₃, —CHF₂, CN, NO₂ or halogen.

The compounds of formula 2a.13 are known in the art (WO 03/064417).

Within the scope of the pharmaceutical combinations according to the invention preferred compounds of formula 2a.13 are those wherein
A′ denotes a double-bonded group selected from 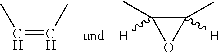
X⁻ denotes chloride, bromide or methanesulphonate, preferably bromide;
R¹⁹ denotes hydroxy or methyl;
R^{1′″} and R^{2′″} which may be identical or different, denote methyl or ethyl, preferably methyl;
R²⁰, R²¹, R^{20′} and R^{21′} which may be identical or different, denote hydrogen, —CF₃, —CHF₂ or fluorine, preferably hydrogen or fluorine.

Within the scope of the pharmaceutical combinations according to the invention particularly preferred compounds of formula 2a.13 are those wherein

A′ denotes a double-bonded group selected from 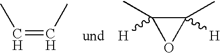
X⁻ denotes bromide;
R¹⁹ denotes hydroxy or methyl, preferably methyl;
R^{1′″} and R^{2′″} which may be identical or different, denote methyl or ethyl, preferably methyl;
R³, R⁴, R^{3′} and R^{4′} which may be identical or different, denote hydrogen or fluorine.

Of particular importance are those medicament combinations which contain in addition to a compound of formula 1 one of the following compounds of formula 2a.13:
tropenol 9-hydroxy-xanthene-9-carboxylate-methobromide (2a.13.1);
scopine 9-hydroxy-xanthene-9-carboxylate methobromide (2a.13.2);
tropenol 9-methyl-xanthene-9-carboxylate-methobromide (2a.13.3);
scopine 9-methyl-xanthene-9-carboxylate-methobromide (2a.13.4);
tropenol 9-ethyl-xanthene-9-carboxylate methobromide (2a.13.5);
tropenol 9-difluoromethyl-xanthene-9-carboxylate-methobromide (2a.13.6);
scopine 9-hydroxymethyl-xanthene-9-carboxylate-methobromide (2a.13.7).

The compounds of formula 2a.13 may optionally be present in the form of the enantiomers, mixtures of enantiomers or racemates thereof, as well as optionally in the form of the hydrates and/or solvates thereof.

Examples of novel pharmaceutical combinations of preferred compounds of formula 1 with the above-mentioned anticholinergics 2a.13 are combinations containing the compounds 1.4 and 2a.13.1; 1.4 and 2a.13.2; 1.4 and 2a.13.3; 1.4 and 2a.13.4; 1.4 and 2a.13.5; 1.4 and 2a.13.6; 1.4 and 2a.13.7; 1.5 and 2a.13.1; 1.5 and 2a.13.2; 1.5 and 2a.13.3; 1.5 and 2a.13.4; 1.5 and 2a.13.5; 1.5 and 2a.13.6; 1.5 and 2a.13.7; 1.6 and 2a.13.1; 1.6 and 2a.13.2; 1.6 and 2a.13.3; 1.6 and 2a.13.4; 1.6 and 2a.13.5; 1.6 and 2a.13.6; 1.6 and 2a.13.7; 1.7 and 2a.13.1; 1.7 and 2a.13.2; 1.7 and 2a.13.3; 1.7 and 2a.13.4; 1.7 and 2a.13.5; 1.7 and 2a.13.6; 1.7 and 2a.13.7; 1.8 and 2a.13.1; 1.8 and 2a.13.2; 1.8 and 2a.13.3; 1.8 and 2a.13.4; 1.8 and 2a.13.5; 1.8 and 2a.13.6; 1.8 and 2a.13.7; 1.9 and 2a.13.1; 1.9 and 2a.13.2; 1.9 and 2a.13.3; 1.9 and 2a.13.4; 1.9 and 2a.13.5; 1.9 and 2a.13.6; 1.9 and 2a.13.7; 1.10 and 2a.13.1; 1.10 and 2a.13.2; 1.10 and 2a.13.3; 1.10 and 2a.13.4; 1.10 and 2a.13.5; 1.10 and 2a.13.6; 1.10 and 2a.13.7; 1.11 and 2a.13.1; 1.11 and 2a.13.2; 1.11 and 2a.13.3; 1.11 and 2a.13.4; 1.11 and 2a.13.5; 1.11 and 2a.13.6; 1.11 and 2a.13.7; 1.12 and 2a.13.1; 1.12 and 2a.13.2; 1.12 and 2a.13.3; 1.12 and 2a.13.4; 1.12 and 2a.13.5; 1.12 and 2a.13.6; 1.12 and 2a.13.7; 1.13 and 2a.13.1; 1.13 and 2a.13.2; 1.13 and 2a.13.3; 1.13 and 2a.13.4; 1.13 and 2a.13.5; 1.13 and 2a.13.6; 1.13 and 2a.13.7; 1.14 and 2a.13.1; 1.14 and 2a.13.2; 1.14 and 2a.13.3; 1.14 and 2a.13.4; 1.14 and 2a.13.5; 1.14 and 2a.13.6; 1.14 and 2a.13.7; in each case optionally in the form of the racemates, enantiomers or diastereomers thereof and optionally in the form of the pharmacologically acceptable acid addition salts, solvates and/or hydrates thereof.

Of the above-mentioned combinations the preferred ones according to the invention are those that contain as compound of formula 1 one of the compounds 1.7, 1.8, 1.9, 1.10, 1.11, 1.12, 1.13, or 1.14. Of the above-mentioned combinations the preferred ones according to the invention are also those which contain as compound 2a.11 one of the compounds 2a.13.2, 2a.13.3, 2a.13.4 or 2a.13.5, while those combinations which contain the compounds 2a.13.3 or 2a.13.4 are of particular importance according to the invention.

Within the scope of the present invention any reference to anticholinergics 1′ is to be taken as being a reference to the pharmacologically active cations of the salts in question. These cations are tiotropium (2a.1′), oxitropium (2a.2′), flutropium (2a.3′), ipratropium (2a.4′), glycopyrronium (2a.5′), trospium (2a.6′) and the cations listed below:

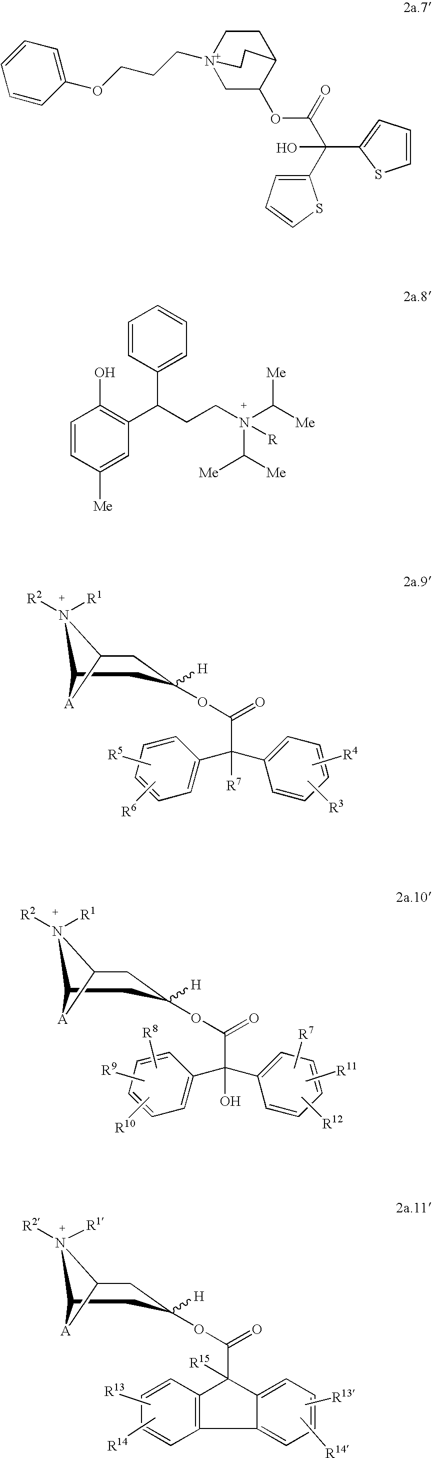

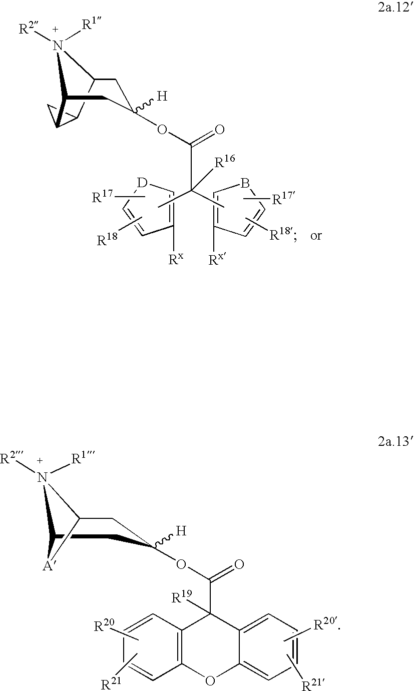

Other preferred medicament combinations according to the invention contain as a further active substance one or more, preferably one PDE IV inhibitor 2b in addition to one or more, preferably one compound of formula 1, optionally in combination with pharmaceutically acceptable excipients.

In medicament combinations of this kind the PDE IV inhibitor 2b is preferably selected from among enprofyllin, theophyllin, roflumilast, ariflo (cilomilast), CP-325,366, BY343, D-4396 (Sch-351591), AWD-12-281 (GW-842470), N-(3,5-dichloro-1-oxo-pyridin-4-yl)-4-difluoromethoxy-3-cyclopropylmethoxybenzamide, NCS-613, pumafentine, (−)p-[(4aR*,10bS*)-9-ethoxy-1,2,3,4,4a,10b-hexahydro-8-methoxy-2-methylbenzo[s][1,6]naphthyridin-6-yl]-N,N-diisopropylbenzamide, (R)-(+)-1-(4-bromobenzyl)-4-[(3-cyclopentyloxy)-4-methoxyphenyl]-2-pyrrolidone, 3-(cyclopentyloxy-4-methoxyphenyl)-1-(4-N′-[N-2-cyano-5-methyl-isothioureido]benzyl)-2-pyrrolidone, cis[4-cyano-4-(3-cyclopentyloxy-4-methoxyphenyl)cyclohexane-1-carboxylic acid], 2-carbomethoxy-4-cyano-4-(3-cyclopropylmethoxy-4-difluoromethoxyphenyl)cyclohexan-1-one, cis[4-cyano-4-(3-cyclopropyl methoxy-4-difluoromethoxyphenyl)cyclohexan-1-ol], (R)-(+)-ethyl[4-(3-cyclopentyloxy-4-methoxyphenyl)pyrrolidin-2-ylidene]acetate, (S)-(−)-ethyl[4-(3-cyclopentyloxy-4-methoxyphenyl)pyrrolidin-2-ylidene]acetate, CDP840, Bay-198004, D-4418, PD-168787, T-440, T-2585, arofyllin, atizoram, V-11294A, CI-1018, CDC-801, CDC-3052, D-22888, YM-58997, Z-15370, 9-cyclopentyl-5,6-dihydro-7-ethyl-3-(2-thienyl)-9H-pyrazolo[3,4-c]-1,2,4-triazolo[4,3-a]pyridine and 9-cyclopentyl-5,6-dihydro-7-ethyl-3-(tert-butyl)-9H-pyrazolo[3.4-c]-1,2,4-triazolo[4,3-a]pyridine, optionally in the form of the racemates, enantiomers or diastereomers thereof and optionally in the form of the pharmacologically acceptable acid addition salts, solvates and/or hydrates thereof.

In particularly preferred medicament combinations the PDE IV inhibitor 2b is selected from among enprofyllin (2b.1), roflumilast (2b.2), ariflo (cilomilast) (2b.3), AWD-12-281 (GW-842470) (2b.4), N-(3,5-dichloro-1-oxo-pyridin-4-yl)-4-difluoromethoxy-3-cyclopropylmethoxybenzamide (2b.5), T-440 (2b.6), T-2585 (2b.7), arofyllin (2b.8), cis[4-cyano-4-(3-cyclopentyloxy-4-methoxyphenyl)cyclohexane-1-carboxylic acid] (2b.9), 2-carbomethoxy-4-cyano-4-(3-cyclopropylmethoxy-4-difluoromethoxyphenyl)cyclohexan-1-one (2b.10), cis[4-cyano-4-(3-cyclopropylmethoxy-4-difluoromethoxyphenyl)cyclohexan-1-ol] (2b.11), PD-168787 (2b.12), atizoram (2b.13), V-11294A (2b.14), CI-1018 (2b.15), CDC-801 (2b.16), D-22888 (2b.17), YM-58997 (2b.18), Z-15370 (2b.19), 9-cyclopentyl-5,6-dihydro-7-ethyl-3-(2-thienyl)-9H-pyrazolo[3,4-c]-1,2,4-triazolo[4,3-a]pyridine (2b.20) and 9-cyclopentyl-5,6-dihydro-7-ethyl-3-(tert-butyl)-9H-pyrazolo[3,4-c]-1,2,4-triazolo[4,3-a]pyridine (2b.21), optionally in the form of the racemates, enantiomers or diastereomers thereof and optionally in the form of the pharmacologically acceptable acid addition salts, solvates and/or hydrates thereof.

In particularly preferred medicament combinations the PDE IV inhibitor 2b is selected from among roflumilast (2b.2), ariflo (cilomilast) (2b.3), AWD-12-281 (GW-842470) (2b.4), arofyllin (2b.8), 2-carbomethoxy-4-cyano-4-(3-cyclopropylmethoxy-4-difluoromethoxyphenyl)cyclohexan-1-one (2b.10), cis[4-cyano-4-(3-cyclopropylmethoxy-4-difluoromethoxyphenyl)cyclohexan-1-ol] (2b.11), atizoram (2b.13), Z-15370 (2b.19), 9-cyclopentyl-5,6-dihydro-7-ethyl-3-(2-thienyl)-9H-pyrazolo[3,4-c]-1,2,4-triazolo[4,3-a]pyridine (2b.20) and 9-cyclopentyl-5,6-dihydro-7-ethyl-3-(tert-butyl)-9H-pyrazolo[3,4-c]-1,2,4-triazolo[4,3-a]pyridine (2b.21), while roflumilast (2b.2), Z-15370 (2b.19) and AWD-12-281 (2b.4) are of particular importance, optionally in the form of the racemates, enantiomers or diastereomers thereof and optionally in the form of the pharmacologically acceptable acid addition salts, solvates and/or hydrates thereof.

By acid addition salts with pharmacologically acceptable acids, which the compounds 2b are optionally capable of forming, are meant for example salts selected from among the hydrochloride, hydrobromide, hydriodide, hydrosulphate, hydrophosphate, hydromethanesulphonate, hydronitrate, hydromaleate, hydroacetate, hydrobenzoate, hydrocitrate, hydrofumarate, hydrotartrate, hydroxalate, hydrosuccinate, hydrobenzoate and hydro-p-toluenesulphonate, preferably hydrochloride, hydrobromide, hydrosulphate, hydrophosphate, hydrofumarate and hydromethanesulphonate.

Examples of novel preferred pharmaceutical combinations of preferred compounds of formula 1 with the above-mentioned PDE IV-inhibitors 2b are combinations containing the compounds 1.4 and 2b.1; 1.4 and 2b.2; 1.4 and 2b.3; 1.4 and 2b.4; 1.4 and 2b.5; 1.4 and 2b.6; 1.4 and 2b.7; 1.4 and 2b.8; 1.4 and 2b.9; 1.4 and 2b.10; 1.4 and 2b.11; 1.4 and 2b.12; 1.4 and 2b.13; 1.4 and 2b.14; 1.4 and 2b.15; 1.4 and 2b.16; 1.4 and 2b.17; 1.4 and 2b.18; 1.4 and 2b.19; 1.4 and 2b.20; 1.4 and 2b.21; 1.5 and 2b.1; 1.5 and 2b.2; 1.5 and 2b.3; 1.5 and 2b.4; 1.5 and 2b.5; 1.5 and 2b.6; 1.5 and 2b.7; 1.5 and 2b.8; 1.5 and 2b.9; 1.5 and 2b.10; 1.5 and 2b.11; 1.5 and 2b.12; 1.5 and 2b.13; 1.5 and 2b.14; 1.5 and 2b.15; 1.5 and 2b.16; 1.5 and 2b.17; 1.5 and 2b.18; 1.5 and 2b.19; 1.5 and 2b.20; 1.5 and 2b.21; 1.6 and 2b.1; 1.6 and 2b.2; 1.6 and 2b.3; 1.6 and 2b.4; 1.6 and 2b.5; 1.6 and 2b.6; 1.6 and 2b.7; 1.6 and 2b.8; 1.6 and 2b.9; 1.6 and 2b.10; 1.6 and 2b.11; 1.6 and 2b.12; 1.6 and 2b.13; 1.6 and 2b.14; 1.6 and 2b.15; 1.6 and 2b.16; 1.6 and 2b.17; 1.6 and 2b.18; 1.6 and 2b.19; 1.6 and 2b.20; 1.6 and 2b.21; 1.7 and 2b.1; 1.7 and 2b.2; 1.7 and 2b.3; 1.7 and 2b.4; 1.7 and 2b.5; 1.7 and 2b.6; 1.7 and 2b.7; 1.7 and 2b.8; 1.7 and 2b.9; 1.7 and 2b.10; 1.7 and 2b.11; 1.7 and 2b.12; 1.7 and 2b.13; 1.7 and 2b.14; 1.7 and 2b.15; 1.7 and 2b.16; 1.7 and 2b.17; 1.7 and 2b.18; 1.7 and 2b.19; 1.7 and 2b.20; 1.7 and 2b.21; 1.8 and 2b.1; 1.8 and 2b.2; 1.8 and 2b.3; 1.8 and 2b.4; 1.8 and 2b.5; 1.8 and 2b.6; 1.8 and 2b.7; 1.8 and 2b.8; 1.8 and 2b.9; 1.8 and 2b.10; 1.8 and 2b.11; 1.8 and 2b.12; 1.8 and 2b.13; 1.8 and 2b.14; 1.8 and 2b.15; 1.8 and 2b.16; 1.8 and 2b.17; 1.8 and 2b.18; 1.8 and 2b.19; 1.8 and 2b.20; 1.8 and 2b.21; 1.9 and 2b.1; 1.9 and 2b.2; 1.9 and 2b.3; 1.9 and 2b.4; 1.9 and 2b.5; 1.9 and 2b.6; 1.9 and 2b.7; 1.9 and 2b.8; 1.9 and 2b.9; 1.9 and 2b.10; 1.9 and 2b.11; 1.9 and 2b.12; 1.9 and 2b.13; 1.9 and 2b.14; 1.9 and 2b.15; 1.9 and 2b.16; 1.9 and 2b.17; 1.9 and 2b.18; 1.9 and 2b.19; 1.9 and 2b.20; 1.9 and 2b.21; 1.10 and 2b.1; 1.10 and 2b.2; 1.10 and 2b.3; 1.10 and 2b.4; 1.10 and 2b.5; 1.10 and 2b.6; 1.10 and 2b.7; 1.10 and 2b.8; 1.10 and 2b.9; 1.10 and 2b.10; 1.10 and 2b.11; 1.10 and 2b.12; 1.10 and 2b.13; 1.10 and 2b.14; 1.10 and 2b.15; 1.10 and 2b.16; 1.10 and 2b.17; 1.10 and 2b.18; 1.10 and 2b.19; 1.10 and 2b.20; 1.10 and 2b.21; 1.11 and 2b.1; 1.11 and 2b.2; 1.11 and 2b.3; 1.11 and 2b.4; 1.11 and 2b.5; 1.11 and 2b.6; 1.11 and 2b.7; 1.11 and 2b.8; 1.11 and 2b.9; 1.11 and 2b.10; 1.11 and 2b.11; 1.11 and 2b.12; 1.11 and 2b.13; 1.11 and 2b.14; 1.11 and 2b.15; 1.11 and 2b.16; 1.11 and 2b.17; 1.11 and 2b.18; 1.11 and 2b.19; 1.11 and 2b.20; 1.11 and 2b.21; 1.12 and 2b.1; 1.12 and 2b.2; 1.12 and 2b.3; 1.12 and 2b.4; 1.12 and 2b.5; 1.12 and 2b.6; 1.12 and 2b.7; 1.12 and 2b.8; 1.12 and 2b.9; 1.12 and 2b.10; 1.12 and 2b.11; 1.12 and 2b.12; 1.12 and 2b.13; 1.12 and 2b.14; 1.12 and 2b.15; 1.12 and 2b.16; 1.12 and 2b.17; 1.12 and 2b.18; 1.12 and 2b.19; 1.12 and 2b.20; 1.12 and 2b.21; 1.13 and 2b.1; 1.13 and 2b.2; 1.13 and 2b.3; 1.13 and 2b.4; 1.13 and 2b.5; 1.13 and 2b.6; 1.13 and 2b.7; 1.13 and 2b.8; 1.13 and 2b.9; 1.13 and 2b.10; 1.13 and 2b.11; 1.13 and 2b.12; 1.13 and 2b.13; 1.13 and 2b.14; 1.13 and 2b.15; 1.13 and 2b.16; 1.13 and 2b.17; 1.13 and 2b.18; 1.13 and 2b.19; 1.13 and 2b.20; 1.13 and 2b.21; 1.14 and 2b.1; 1.14 and 2b.2; 1.14 and 2b.3; 1.14 and 2b.4; 1.14 and 2b.5; 1.14 and 2b.6; 1.14 and 2b.7; 1.14 and 2b.8; 1.14 and 2b.9; 1.14 and 2b.10; 1.14 and 2b.11; 1.14 and 2b.12; 1.14 and 2b.13; 1.14 and 2b.14; 1.14 and 2b.15; 1.14 and 2b.16; 1.14 and 2b.17; 1.14 and 2b.18; 1.14 and 2b.19; 1.14 and 2b.20; 1.14 and 2b.21; in each case optionally in the form of the racemates, enantiomers or diastereomers thereof and optionally in the form of the pharmacologically acceptable acid addition salts, solvates and/or hydrates thereof.

Of the above-mentioned combinations the preferred ones according to the invention are those that contain as compound of formula 1 one of the compounds 1.7, 1.8, 1.9, 1.10, 1.11, 1.12, 1.13, or 1.14. Of the above-mentioned combinations the preferred ones according to the invention are also those which contain as compound 2b one of the compounds 2b.2, 2b.3, 2b.4, 2b.8, 2b.10, 2b.11, 2b.13, 2b.19, 2b.20 or 2b.21, while those combinations that contain one of the compounds 2b.2, 2b.4 or 2b.19 are of particular importance according to the invention.

Other preferred medicament combinations according to the invention contain as a further active substance one or more, preferably one steroid 2c in addition to one or more, preferably one, compound of formula 1, optionally in combination with pharmaceutically acceptable excipients.

In medicament combinations of this kind the steroid 2c is preferably selected from among prednisolone (2c.1), prednisone (2c.2), butixocortpropionate (2c.3), RPR-106541 (2c.4), flunisolide (2c.5), beclomethasone (2c.6), triamcinolone (2c.7), budesonide (2c.8), fluticasone (2c.9), mometasone (2c.10), ciclesonide (2c.11), rofleponide (2c.12), ST-126 (2c.13), dexamethasone (2c.14), (S)-fluoromethyl 6α,9α-difluoro-17α-[(2-furanylcarbonyl)oxy]-11β-hydroxy-16α-methyl-3-oxo-androsta-1,4-diene-17β-carbothionate (2c.15), (S)-(2-oxo-tetrahydro-furan-3S-yl) 6α,9α-difluoro-11β-hydroxy-16α-methyl-3-oxo-17α-propionyloxy-androsta-1,4-diene-17β-carbothionate (2c.16) and etiprednol-dichloroacetate (BNP-166, 2c.17), optionally in the form of the racemates, enantiomers or diastereomers thereof and optionally in the form of the salts and derivatives thereof, the solvates and/or hydrates thereof.

In particularly preferred medicament combinations the steroid 2c is selected from among flunisolide (2c.5), beclomethasone (2c.6), triamcinolone (2c.7), budesonide (2c.8), fluticasone (2c.9), mometasone (2c.10), ciclesonide (2c.11), rofleponide (2c.12), ST-126 (2c.13), dexamethasone (2c.14), (S)-fluoromethyl 6α,9α-difluoro-17α-[(2-furanylcarbonyl)oxy]-11β-hydroxy-16α-methyl-3-oxo-androsta-1,4-diene-17β-carbothionate (2c.15), (S)-(2-oxo-tetrahydro-furan-3S-yl) 6α,9α-difluoro-11β-hydroxy-16α-methyl-3-oxo-17α-propionyloxy-androsta-1,4-diene-17β-carbothionate (2c.16) and etiprednol-dichloroacetate (2c.17), optionally in the form of the racemates, enantiomers or diastereomers thereof and optionally in the form of the salts and derivatives thereof, the solvates and/or hydrates thereof.

In particularly preferred medicament combinations the steroid 2c is selected from among budesonide (2c.8), fluticasone (2c.9), mometasone (2c.10), ciclesonide (2c.11), (S)-fluoromethyl 6α,9α-difluoro-17β-[(2-furanylcarbonyl)oxy]-11β-hydroxy-16α-methyl-3-oxo-androsta-1,4-diene-17β-carbothionate (2c.15) and etiprednol-dichloroacetate (2c.17), optionally in the form of the racemates, enantiomers or diastereomers thereof and optionally in the form of the salts and derivatives thereof, the solvates and/or hydrates thereof.

Any reference to steroids 2c includes a reference to any salts or derivatives, hydrates or solvates thereof which may exist. Examples of possible salts and derivatives of the steroids 2c may be: alkali metal salts, such as for example sodium or potassium salts, sulphobenzoates, phosphates, isonicotinates, acetates, propionates, dihydrogen phosphates, palmitates, pivalates or also furoates.

Examples of novel preferred pharmaceutical combinations of preferred compounds of formula 1 with the above-mentioned steroids 2c are combinations containing the compounds 1.4 and 2c.1; 1.4 and 2c.2; 1.4 and 2c.3; 1.4 and 2c.4; 1.4 and 2c.5; 1.4 and 2c.6; 1.4 and 2c.7; 1.4 and 2c.8; 1.4 and 2c.9; 1.4 and 2c.10; 1.4 and 2c.11; 1.4 and 2c.12; 1.4 and 2c.13; 1.4 and 2c.14; 1.4 and 2c.15; 1.4 and 2c.16; 1.4 and 2c.17; 1.5 and 2c.1; 1.5 and 2c.2; 1.5 and 2c.3; 1.5 and 2c.4; 1.5 and 2c.5; 1.5 and 2c.6; 1.5 and 2c.7; 1.5 and 2c.8; 1.5 and 2c.9; 1.5 and 2c.10; 1.5 and 2c.11; 1.5 and 2c.12; 1.5 and 2c.13; 1.5 and 2c.14; 1.5 and 2c.15; 1.5 and 2c.16; 1.5 and 2c.17; 1.6 and 2c.1; 1.6 and 2c.2; 1.6 and 2c.3; 1.6 and 2c.4; 1.6 and 2c.5; 1.6 and 2c.6; 1.6 and 2c.7; 1.6 and 2c.8; 1.6 and 2c.9; 1.6 and 2c.10; 1.6 and 2c.11; 1.6 and 2c.12; 1.6 and 2c.13; 1.6 and 2c.14; 1.6 and 2c.15; 1.6 and 2c.16; 1.6 and 2c.17; 1.7 and 2c.1; 1.7 and 2c.2; 1.7 and 2c.3; 1.7 and 2c.4; 1.7 and 2c.5; 1.7 and 2c.6; 1.7 and 2c.7; 1.7 and 2c.8; 1.7 and 2c.9; 1.7 and 2c.10; 1.7 and 2c.11; 1.7 and 2c.12; 1.7 and 2c.13; 1.7 and 2c.14; 1.7 and 2c.15; 1.7 and 2c.16; 1.7 and 2c.17; 1.8 and 2c.1; 1.8 and 2c.2; 1.8 and 2c.3; 1.8 and 2c.4; 1.8 and 2c.5; 1.8 and 2c.6; 1.8 and 2c.7; 1.8 and 2c.8; 1.8 and 2c.9; 1.8 and 2c.10; 1.8 and 2c.11; 1.8 and 2c.12; 1.8 and 2c.13; 1.8 and 2c.14; 1.8 and 2c.15; 1.8 and 2c.16; 1.8 and 2c.17; 1.9 and 2c.1; 1.9 and 2c.2; 1.9 and 2c.3; 1.9 and 2c.4; 1.9 and 2c.5; 1.9 and 2c.6; 1.9 and 2c.7; 1.9 and 2c.8; 1.9 and 2c.9; 1.9 and 2c.10; 1.9 and 2c.11; 1.9 and 2c.12; 1.9 and 2c.13; 1.9 and 2c.14; 1.9 and 2c.15; 1.9 and 2c.16; 1.9 and 2c.17; 1.10 and 2c.1; 1.10 and 2c.2; 1.10 and 2c.3; 1.10 and 2c.4; 1.10 and 2c.5; 1.10 and 2c.6; 1.10 and 2c.7; 1.10 and 2c.8; 1.10 and 2c.9; 1.10 and 2c.10; 1.10 and 2c.11; 1.10 and 2c.12; 1.10 and 2c.13; 1.10 and 2c.14; 1.10 and 2c.15; 1.10 and 2c.16; 1.10 and 2c.17; 1.11 and 2c.1; 1.11 and 2c.2; 1.11 and 2c.3; 1.11 and 2c.4; 1.11 and 2c.5; 1.11 and 2c.6; 1.11 and 2c.7; 1.11 and 2c.8; 1.11 and 2c.9; 1.11 and 2c.10; 1.11 and 2c.11; 1.11 and 2c.12; 1.11 and 2c.13; 1.11 and 2c.14; 1.11 and 2c.15; 1.11 and 2c.16; 1.11 and 2c.17; 1.12 and 2c.1; 1.12 and 2c.2; 1.12 and 2c.3; 1.12 and 2c.4; 1.12 and 2c.5; 1.12 and 2c.6; 1.12 and 2c.7; 1.12 and 2c.8; 1.12 and 2c.9; 1.12 and 2c.10; 1.12 and 2c.11; 1.12 and 2c.12; 1.12 and 2c.13; 1.12 and 2c.14; 1.12 and 2c.15; 1.12 and 2c.16; 1.12 and 2c.17; 1.13 and 2c.1; 1.13 and 2c.2; 1.13 and 2c.3; 1.13 and 2c.4; 1.13 and 2c.5; 1.13 and 2c.6; 1.13 and 2c.7; 1.13 and 2c.8; 1.13 and 2c.9; 1.13 and 2c.10; 1.13 and 2c.11; 1.13 and 2c.12; 1.13 and 2c.13; 1.13 and 2c.14; 1.13 and 2c.15; 1.13 and 2c.16; 1.13 and 2c.17; 1.14 and 2c.1; 1.14 and 2c.2; 1.14 and 2c.3; 1.14 and 2c.4; 1.14 and 2c.5; 1.14 and 2c.6; 1.14 and 2c.7; 1.14 and 2c.8; 1.14 and 2c.9; 1.14 and 2c.10; 1.14 and 2c.11; 1.14 and 2c.12; 1.14 and 2c.13; 1.14 and 2c.14; 1.14 and 2c.15; 1.14 and 2c.16; 1.14 and 2c.17; in each case optionally in the form of the racemates, enantiomers or diastereomers thereof and optionally in the form of the pharmacologically acceptable acid addition salts, solvates and/or hydrates thereof.

Of the above-mentioned combinations the preferred ones according to the invention are those that contain as compound of formula 1 one of the compounds 1.7, 1.8, 1.9, 1.10, 1.11, 1.12, 1.13, or 1.14. Of the above-mentioned combinations the preferred ones according to the invention are also those which contain as compound 2c one of the compounds 2c.5, 2c.6, 2c.7, 2c.8, 2c.9, 2c.10, 2c.11, 2c.12, 2c.13, 2c.14, 2c.15, 2c.16 or 2c.17, while those combinations that contain one of the compounds 2c.8, 2c.9, 2c.10, 2c.11, 2c.15 or 2c.17 are of particular importance according to the invention.

Other preferred medicament combinations according to the invention contain, as an additional active substance, one or more, preferably one, LTD4 antagonist 2d in addition to one or more, preferably one compound of formula 1, optionally in combination with pharmaceutically acceptable excipients.

In such medicament combinations the LTD4 antagonist 2d is preferably selected from among montelukast (2d.1), 1-(((R)-(3-(2-(6,7-difluoro-2-quinolinyl)ethenyl)phenyl)-3-(2-(2-hydroxy-2-propyl)phenyl)thio)methylcyclopropane-acetic acid (2d.2), 1-(((1(R)-3(3-(2-(2,3-dichlorothieno[3,2-b]pyridin-5-yl)-(E)-ethenyl)phenyl)-3-(2-(1-hydroxy-1-methylethyl)phenyl)propyl)thio)methyl)cyclopropancetic acid (2d.3), pranlukast (2d.4), zafirlukast (2d.5), [2-[[2-(4-tert-butyl-2-thiazolyl)-5-benzofuranyl]oxymethyl]-phenyl]acetic acid (2d.6), MCC-847 (ZD-3523) (2d.7), MN-001 (2d.8), MEN-91507 (LM-1507) (2d.9), VUF-5078 (2d.10), VUF-K-8707 (2d.11) and L-733321 (2d.12), optionally in the form of the racemates, enantiomers or diastereomers thereof, optionally in the form of the pharmacologically acceptable acid addition salts thereof as well as optionally in the form of the salts and derivatives thereof, the solvates and/or hydrates thereof.

In preferred medicament combinations the LTD4 antagonist 2d is selected from the group comprising montelukast (2d.1), pranlukast (2d.4), zafirlukast (2d.5), MCC-847 (ZD-3523) (2d.7), MN-001 (2d.8), MEN-91507 (LM-1507) (2d.9), VUF-5078 (2d.10), VUF-K-8707 (2d.11) and L-733321 (2d.12), optionally in the form of the racemates, enantiomers or diastereomers thereof, optionally in the form of the pharmacologically acceptable acid addition salts thereof as well as optionally in the form of the salts and derivatives thereof, the solvates and/or hydrates thereof.

In particularly preferred medicament combinations the LTD4 antagonist 2d is selected from the group comprising montelukast (2d.1), pranlukast (2d.4), zafirlukast (2d.5), MCC-847 (ZD-3523) (2d.7), MN-001 (2d.8) and MEN-91507 (LM-1507) (2d.9), while montelukast (2d.1), pranlukast (2d.4) and zafirlukast (2d.5) are particularly preferred, optionally in the form of the racemates, enantiomers or diastereomers thereof, optionally in the form of the pharmacologically acceptable acid addition salts thereof as well as optionally in the form of the salts and derivatives thereof, the solvates and/or hydrates thereof.

By the acid addition salts with pharmacologically acceptable acids which the compounds 2d may possibly be capable of forming are meant for example salts selected from the group comprising the hydrochloride, hydrobromide, hydriodide, hydrosulphate, hydrophosphate, hydromethanesulphonate, hydronitrate, hydromaleate, hydroacetate, hydrobenzoate, hydrocitrate, hydrofumarate, hydrotartrate, hydroxalate, hydrosuccinate, hydrobenzoate and hydro-p-toluenesulphonate, preferably the hydrochloride, hydrobromide, hydrosulphate, hydrophosphate, hydrofumarate and hydromethanesulphonate.

Examples of possible salts and derivatives which the compounds 2d may possibly be capable of forming include for example: alkali metal salts, such as for example sodium or potassium salts, alkaline earth metal salts, sulphobenzoates, phosphates, isonicotinates, acetates, propionates, dihydrogen phosphates, palmitates, pivalates or furoates.

Examples of novel preferred pharmaceutical combinations of preferred compounds of formula 1 with the above-mentioned LTD4-antagonists 2d are combinations containing the compounds 1.4 and 2d.1; 1.4 and 2d.2; 1.4 and 2d.3; 1.4 and 2d.4; 1.4 and 2d.5; 1.4 and 2d.6; 1.4 and 2d.7; 1.4 and 2d.8; 1.4 and 2d.9; 1.4 and 2d.10; 1.4 and 2d.11; 1.4 and 2d.12; 1.5 and 2d.1; 1.5 and 2d.2; 1.5 and 2d.3; 1.5 and 2d.4; 1.5 and 2d.5; 1.5 and 2d.6; 1.5 and 2d.7; 1.5 and 2d.8; 1.5 and 2d.9; 1.5 and 2d.10; 1.5 and 2d.11; 1.5 and 2d.12; 1.6 and 2d.1; 1.6 and 2d.2; 1.6 and 2d.3; 1.6 and 2d.4; 1.6 and 2d.5; 1.6 and 2d.6; 1.6 and 2d.7; 1.6 and 2d.8; 1.6 and 2d.9; 1.6 and 2d.10; 1.6 and 2d.11; 1.6 and 2d.12; 1.7 and 2d.1; 1.7 and 2d.2; 1.7 and 2d.3; 1.7 and 2d.4; 1.7 and 2d.5; 1.7 and 2d.6; 1.7 and 2d.7; 1.7 and 2d.8; 1.7 and 2d.9; 1.7 and 2d.10; 1.7 and 2d.11; 1.7 and 2d.12; 1.8 and 2d.1; 1.8 and 2d.2; 1.8 and 2d.3; 1.8 and 2d.4; 1.8 and 2d.5; 1.8 and 2d.6; 1.8 and 2d.7; 1.8 and 2d.8; 1.8 and 2d.9; 1.8 and 2d.10; 1.8 and 2d.11; 1.8 and 2d.12; 1.9 and 2d.1; 1.9 and 2d.2; 1.9 and 2d.3; 1.9 and 2d.4; 1.9 and 2d.5; 1.9 and 2d.6; 1.9 and 2d.7; 1.9 and 2d.8; 1.9 and 2d.9; 1.9 and 2d.10; 1.9 and 2d.11; 1.9 and 2d.12; 1.10 and 2d.1; 1.10 and 2d.2; 1.10 and 2d.3; 1.10 and 2d.4; 1.10 and 2d.5; 1.10 and 2d.6; 1.10 and 2d.7; 1.10 and 2d.8; 1.10 and 2d.9; 1.10 and 2d.10; 1.10 and 2d.11; 1.10 and 2d.12; 1.11 and 2d.1; 1.11 and 2d.2; 1.11 and 2d.3; 1.11 and 2d.4; 1.11 and 2d.5; 1.11 and 2d.6; 1.11 and 2d.7; 1.11 and 2d.8; 1.11 and 2d.9; 1.11 and 2d.10; 1.11 and 2d.11; 1.11 and 2d.12; 1.12 and 2d.1; 1.12 and 2d.2; 1.12 and 2d.3; 1.12 and 2d.4; 1.12 and 2d.5; 1.12 and 2d.6; 1.12 and 2d.7; 1.12 and 2d.8; 1.12 and 2d.9; 1.12 and 2d.10; 1.12 and 2d.11; 1.12 and 2d.12; 1.13 and 2d.1; 1.13 and 2d.2; 1.13 and 2d.3; 1.13 and 2d.4; 1.13 and 2d.5; 1.13 and 2d.6; 1.13 and 2d.7; 1.13 and 2d.8; 1.13 and 2d.9; 1.13 and 2d.10; 1.13 and 2d.11; 1.13 and 2d.12; 1.14 and 2d.1; 1.14 and 2d.2; 1.14 and 2d.3; 1.14 and 2d.4; 1.14 and 2d.5; 1.14 and 2d.6; 1.14 and 2d.7; 1.14 and 2d.8; 1.14 and 2d.9; 1.14 and 2d.10; 1.14 and 2d.11; 1.14 and 2d.12; in each case optionally in the form of the racemates, enantiomers or diastereomers thereof and optionally in the form of the pharmacologically acceptable acid addition salts, solvates and/or hydrates thereof.

Of the above-mentioned combinations the preferred ones according to the invention are those that contain as compound of formula 1 one of the compounds 1.7, 1.8, 1.9, 1.10, 1.11, 1.12, 1.13, or 1.14. Of the above-mentioned combinations the preferred ones according to the invention are also those which contain as compound 2d one of the compounds 2d.1, 2d.4, 2d.5, 2d.7, 2d.8, 2d.9, 2d.10, 2d.11 or 2d.12, while those combinations that contain one of the compounds 2d.1, 2d.4, 2d.5, 2d.7, 2d.8 or 2d.9 are particularly important according to the invention, while those combinations that contain one of the compounds 2d.1, 2d.4 or 2d.5 are of exceptional importance.

Other preferred medicament combinations according to the invention contain one or more, preferably one, EGFR-inhibitor 2e as an additional active substance in addition to one or more, preferably one compound of formula 1, optionally in combination with pharmaceutically acceptable excipients.

In such medicament combinations the EGFR-inhibitor 2e is selected for example from the group comprising 4-[(3-chloro-4-fluorophenyl)amino]-6-{[4-(morpholin-4-yl)-1-oxo-2-buten-1-yl]amino}-7-cyclopropylmethoxy-quinazoline, 4-[(3-chloro-4-fluorophenyl)amino]-6-{[4-(N,N-diethylamino)-1-oxo-2-buten-1-yl]amino}-7-cyclopropylmethoxy-quinazoline, 4-[(3-chloro-4-fluorophenyl)amino]-6-{[4-(N,N-dimethylamino)-1-oxo-2-buten-1-yl]amino}-7-cyclopropylmethoxy-quinazoline, 4-[(R)-(1-phenyl-ethyl)amino]-6-{[4-(morpholin-4-yl)-1-oxo-2-buten-1-yl]amino}-7-cyclopentyloxy-quinazoline, 4-[(3-chloro-4-fluoro-phenyl)amino]-6-{[4-((R)-6-methyl-2-oxo-morpholin-4-yl)-1-oxo-2-buten-1-yl]amino}-7-cyclopropylmethoxy-quinazoline, 4-[(3-chloro-4-fluoro-phenyl)amino]-6-{[4-((R)-6-methyl-2-oxo-morpholin-4-yl)-1-oxo-2-buten-1-yl]amino}-7-[(S)-(tetrahydrofuran-3-yl)oxy]-quinazoline, 4-[(3-chloro-4-fluoro-phenyl)amino]-6-{[4-((R)-2-methoxymethyl-6-oxo-morpholin-4-yl)-1-oxo-2-buten-1-yl]amino}-7-cyclopropylmethoxy-quinazoline, 4-[(3-chloro-4-fluoro-phenyl)amino]-6-[2-((S)-6-methyl-2-oxo-morpholin-4-yl)-ethoxy]-7-methoxy-quinazoline, 4-[(3-chloro-4-fluorophenyl)amino]-6-({4-[N-(2-methoxy-ethyl)-N-methyl-amino]-1-oxo-2-buten-1-yl}amino)-7-cyclopropylmethoxy-quinazoline, 4-[(3-chloro-4-fluorophenyl)amino]-6-{[4-(N,N-dimethylamino)-1-oxo-2-buten-1-yl]amino}-7-cyclopentyloxy-quinazoline, 4-[(R)-(1-phenyl-ethyl)amino]-6-{[4-(N,N-bis-(2-methoxy-ethyl)-amino)-1-oxo-2-buten-1-yl]amino}-7-cyclopropyl methoxy-quinazoline, 4-[(R)-(1-phenyl-ethyl)amino]-6-({4-[N-(2-methoxy-ethyl)-N-ethyl-amino]-1-oxo-2-buten-1-yl}amino)-7-cyclopropylmethoxy-quinazoline, 4-[(R)-(1-phenyl-ethyl)amino]-6-({4-[N-(2-methoxy-ethyl)-N-methyl-amino]-1-oxo-2-buten-1-yl}amino)-7-cyclopropylmethoxy-quinazoline, 4-[(R)-(1-phenyl-ethyl)amino]-6-({4-[N-(tetrahydropyran-4-yl)-N-methyl-amino]-1-oxo-2-buten-1-yl}amino)-7-cyclopropylmethoxy-quinazoline, 4-[(3-chloro-4-fluorophenyl)amino]-6-{[4-(N,N-dimethylamino)-1-oxo-2-buten-1-yl]amino}-7-((R)-tetrahydrofuran-3-yloxy)-quinazoline, 4-[(3-chloro-4-fluorophenyl)amino]-6-{[4-(N,N-dimethylamino)-1-oxo-2-buten-1-yl]amino}-7-((S)-tetrahydrofuran-3-yloxy)-quinazoline, 4-[(3-chloro-4-fluorophenyl)amino]-6-({4-[N-(2-methoxy-ethyl)-N-methyl-amino]-1-oxo-2-buten-1-yl}amino)-7-cyclopentyloxy-quinazoline, 4-[(3-chloro-4-fluorophenyl)amino]-6-{[4-(N-cyclopropyl-N-methyl-amino)-1-oxo-2-buten-1-yl]amino}-7-cyclopentyloxy-quinazoline, 4-[(3-chloro-4-fluorophenyl)amino]-6-{[4-(N,N-dimethylamino)-1-oxo-2-buten-1-yl]amino}-7-[(R)-(tetrahydrofuran-2-yl)methoxy]-quinazoline, 4-[(3-chloro-4-fluorophenyl)amino]-6-{[4-(N,N-dimethylamino)-1-oxo-2-buten-1-yl]amino}-7-[(S)-(tetrahydrofuran-2-yl)methoxy]-quinazoline, 4-[(3-ethynyl-phenyl)amino]-6,7-bis-(2-methoxy-ethoxy)-quinazoline, 4-[(3-chloro-4-fluorophenyl)amino]-7-[3-(morpholin-4-yl)-propyloxy]-6-[(vinyl-carbonyl)amino]-quinazoline, 4-[(R)-(1-phenyl-ethyl)amino]-6-(4-hydroxy-phenyl)-7H-pyrrolo[2,3-d]pyrimidine, 3-cyano-4-[(3-chloro-4-fluorophenyl)amino]-6-{[4-(N,N-dimethylamino)-1-oxo-2-buten-1-yl]amino}-7-ethoxy-quinoline, 4-{[3-chloro-4-(3-fluoro-benzyloxy)-phenyl]amino}-6-(5-{[(2-methanesulphonyl-ethyl)amino]methyl}-furan-2-yl)quinazoline, 4-[(R)-(1-phenyl-ethyl)amino]-6-{[4-((R)-6-methyl-2-oxo-morpholin-4-yl)-1-oxo-2-buten-1-yl]amino}-7-methoxy-quinazoline, 4-[(3-chloro-4-fluorophenyl)amino]-6-{[4-(morpholin-4-yl)-1-oxo-2-buten-1-yl]amino}-7-[(tetrahydrofuran-2-yl)methoxy]-quinazoline, 4-[(3-chloro-4-fluorophenyl)amino]-6-({4-[N,N-bis-(2-methoxy-ethyl)-amino]-1-oxo-2-buten-1-yl}amino)-7-[(tetrahydrofuran-2-yl)methoxy]-quinazoline, 4-[(3-ethynyl-phenyl)amino]-6-{[4-(5,5-dimethyl-2-oxo-morpholin-4-yl)-1-oxo-2-buten-1-yl]amino}-quinazoline, 4-[(3-chloro-4-fluoro-phenyl)amino]-6-[2-(2,2-dimethyl-6-oxo-morpholin-4-yl)-ethoxy]-7-methoxy-quinazoline, 4-[(3-chloro-4-fluoro-phenyl)amino]-6-[2-(2,2-dimethyl-6-oxo-morpholin-4-yl)-ethoxy]-7-[(R)-(tetrahydrofuran-2-yl)methoxy]-quinazoline, 4-[(3-chloro-4-fluoro-phenyl)amino]-7-[2-(2,2-dimethyl-6-oxo-morpholin-4-yl)-ethoxy]-6-[(S)-(tetrahydrofuran-2-yl)methoxy]-quinazoline, 4-[(3-chloro-4-fluoro-phenyl)amino]-6-{2-[4-(2-oxo-morpholin-4-yl)-piperidin-1-yl]-ethoxy}-7-methoxy-quinazoline, 4-[(3-chloro-4-fluoro-phenyl)amino]-6-[1-(tert-butyloxycarbonyl)-piperidin-4-yloxy]-7-methoxy-quinazoline, 4-[(3-chloro-4-fluoro-phenyl)amino]-6-(trans-4-amino-cyclohexan-1-yloxy)-7-methoxy-quinazoline, 4-[(3-chloro-4-fluoro-phenyl)amino]-6-(trans-4-methanesulphonylamino-cyclohexan-1-yloxy)-7-methoxy-quinazoline, 4-[(3-chloro-4-fluoro-phenyl)amino]-6-(tetrahydropyran-3-yloxy)-7-methoxy-quinazoline, 4-[(3-chloro-4-fluoro-phenyl)amino]-6-(1-methyl-piperidin-4-yloxy)-7-methoxy-quinazoline, 4-[(3-chloro-4-fluoro-phenyl)amino]-6-{1-[(morpholin-4-yl)carbonyl]-piperidin-4-yloxy}-7-methoxy-quinazoline, 4-[(3-chloro-4-fluoro-phenyl)amino]-6-{1-[(methoxymethyl)carbonyl]-piperidin-4-yloxy}-7-methoxy-quinazoline, 4-[(3-chloro-4-fluoro-phenyl)amino]-6-(piperidin-3-yloxy)-7-methoxy-quinazoline, 4-[(3-chloro-4-fluoro-phenyl)amino]-6-[1-(2-acetylamino-ethyl)-piperidin-4-yloxy]-7-methoxy-quinazoline, 4-[(3-chloro-4-fluoro-phenyl)amino]-6-(tetrahydropyran-4-yloxy)-7-ethoxy-quinazoline, 4-[(3-chloro-4-fluoro-phenyl)amino]-6-((S)-tetrahydrofuran-3-yloxy)-7-hydroxy-quinazoline, 4-[(3-chloro-4-fluoro-phenyl)amino]-6-(tetrahydropyran-4-yloxy)-7-(2-methoxy-ethoxy)-quinazoline, 4-[(3-chloro-4-fluoro-phenyl)amino]-6-{trans-4-[(dimethylamino)sulphonylamino]-cyclohexan-1-yloxy}-7-methoxy-quinazoline, 4-[(3-chloro-4-fluoro-phenyl)amino]-6-{trans-4-[(morpholin-4-yl)carbonylamino]-cyclohexan-1-yloxy}-7-methoxy-quinazoline, 4-[(3-chloro-4-fluoro-phenyl)amino]-6-{trans-4-[(morpholin-4-yl)sulphonylamino]-cyclohexan-1-yloxy}-7-methoxy-quinazoline, 4-[(3-chloro-4-fluoro-phenyl)amino]-6-(tetrahydropyran-4-yloxy)-7-(2-acetylamino-ethoxy)-quinazoline, 4-[(3-chloro-4-fluoro-phenyl)amino]-6-(tetrahydropyran-4-yloxy)-7-(2-methanesulphonylamino-ethoxy)-quinazoline, 4-[(3-chloro-4-fluoro-phenyl)amino]-6-{1-[(piperidin-1-yl)carbonyl]-piperidin-4-yloxy}-7-methoxy-quinazoline, 4-[(3-chloro-4-fluoro-phenyl)amino]-6-(1-aminocarbonylmethyl-piperidin-4-yloxy)-7-methoxy-quinazoline, 4-[(3-chloro-4-fluoro-phenyl)amino]-6-(cis-4-{N-[(tetrahydropyran-4-yl)carbonyl]-N-methyl-amino}-cyclohexan-1-yloxy)-7-methoxy-quinazoline, 4-[(3-chloro-4-fluoro-phenyl)amino]-6-(cis-4-{N-[(morpholin-4-yl)carbonyl]-N-methyl-amino}-cyclohexan-1-yloxy)-7-methoxy-quinazoline, 4-[(3-chloro-4-fluoro-phenyl)amino]-6-(cis-4-{N-[(morpholin-4-yl)sulphonyl]-N-methyl-amino}-cyclohexan-1-yloxy)-7-methoxy-quinazoline, 4-[(3-chloro-4-fluoro-phenyl)amino]-6-(trans-4-ethanesulphonylamino-cyclohexan-1-yloxy)-7-methoxy-quinazoline, 4-[(3-chloro-4-fluoro-phenyl)amino]-6-(1-methanesulphonyl-piperidin-4-yloxy)-7-ethoxy-quinazoline, 4-[(3-chloro-4-fluoro-phenyl)amino]-6-(1-methanesulphonyl-piperidin-4-yloxy)-7-(2-methoxy-ethoxy)-quinazoline, 4-[(3-chloro-4-fluoro-phenyl)amino]-6-[1-(2-methoxy-acetyl)-piperidin-4-yloxy]-7-(2-methoxy-ethoxy)-quinazoline, 4-[(3-chloro-4-fluoro-phenyl)amino]-6-(cis-4-acetylamino-cyclohexan-1-yloxy)-7-methoxy-quinazoline, 4-[(3-ethynyl-phenyl)amino]-6-[1-(tert.-butyloxycarbonyl)-piperidin-4-yloxy]-7-methoxy-quinazoline, 4-[(3-ethynyl-phenyl)amino]-6-(tetrahydropyran-4-yloxy]-7-methoxy-quinazoline, 4-[(3-chloro-4-fluoro-phenyl)amino]-6-(cis-4-{N-[(piperidin-1-yl)carbonyl]-N-methyl-amino}-cyclohexan-1-yloxy)-7-methoxy-quinazoline, 4-[(3-chloro-4-fluoro-phenyl)amino]-6-(cis-4-{N-[(4-methyl-piperazin-1-yl)carbonyl]-N-methyl-amino}-cyclohexan-1-yloxy)-7-methoxy-quinazoline, 4-[(3-chloro-4-fluoro-phenyl)amino]-6-{cis-4-[(morpholin-4-yl)carbonylamino]-cyclohexan-1-yloxy}-7-methoxy-quinazoline, 4-[(3-chloro-4-fluoro-phenyl)amino]-6-{1-[2-(2-oxopyrrolidin-1-yl)ethyl]-piperidin-4-yloxy}-7-methoxy-quinazoline, 4-[(3-chloro-4-fluoro-phenyl)amino]-6-{1-[(morpholin-4-yl)carbonyl]-piperidin-4-yloxy}-7-(2-methoxy-ethoxy)-quinazoline, 4-[(3-ethynyl-phenyl)amino]-6-(1-acetyl-piperidin-4-yloxy)-7-methoxy-quinazoline, 4-[(3-ethynyl-phenyl)amino]-6-(1-methyl-piperidin-4-yloxy)-7-methoxy-quinazoline, 4-[(3-ethynyl-phenyl)amino]-6-(1-methanesulphonyl-piperidin-4-yloxy)-7-methoxy-quinazoline, 4-[(3-chloro-4-fluoro-phenyl)amino]-6-(1-methyl-piperidin-4-yloxy)-7(2-methoxy-ethoxy)-quinazoline, 4-[(3-chloro-4-fluoro-phenyl)amino]-6-(1-isopropyloxycarbonyl-piperidin-4-yloxy)-7-methoxy-quinazoline, 4-[(3-chloro-4-fluoro-phenyl)amino]-6-(cis-4-methylamino-cyclohexan-1-yloxy)-7-methoxy-quinazoline, 4-[(3-chloro-4-fluoro-phenyl)amino]-6-{cis-4-[N-(2-methoxy-acetyl)-N-methyl-amino]-cyclohexan-1-yloxy}-7-methoxy-quinazoline, 4-[(3-ethynyl-phenyl)amino]-6-(piperidin-4-yloxy)-7-methoxy-quinazoline, 4-[(3-ethynyl-phenyl)amino]-6-[1-(2-methoxy-acetyl)-piperidin-4-yloxy]-7-methoxy-quinazoline, 4-[(3-ethynyl-phenyl)amino]-6-{1-[(morpholin-4-yl)carbonyl]-piperidin-4-yloxy}-7-methoxy-quinazoline, 4-[(3-chloro-4-fluoro-phenyl)amino]-6-{1-[(cis-2.6-dimethyl-morpholin-4-yl)carbonyl]-piperidin-4-yloxy}-7-methoxy-quinazoline, 4-[(3-chloro-4-fluoro-phenyl)amino]-6-{1-[(2-methyl-morpholin-4-yl)carbonyl]-piperidin-4-yloxy}-7-methoxy-quinazoline, 4-[(3-chloro-4-fluoro-phenyl)amino]-6-{1-[(S,S)-(2-oxa-5-aza-bicyclo[2,2,1]hept-5-yl)carbonyl]-piperidin-4-yloxy}-7-methoxy-quinazoline, 4-[(3-chloro-4-fluoro-phenyl)amino]-6-{1-[(N-methyl-N-2-methoxyethyl-amino)carbonyl]-piperidin-4-yloxy}-7-methoxy-quinazoline, 4-[(3-chloro-4-fluoro-phenyl)amino]-6-(1-ethyl-piperidin-4-yloxy)-7-methoxy-quinazoline, 4-[(3-chloro-4-fluoro-phenyl)amino]-6-{1-[(2-methoxyethyl)carbonyl]-piperidin-4-yloxy}-7-methoxy-quinazoline, 4-[(3-chloro-4-fluoro-phenyl)amino]-6-{1-[(3-methoxypropyl-amino)-carbonyl]-piperidin-4-yloxy}-7-methoxy-quinazoline, 4-[(3-chloro-4-fluoro-phenyl)amino]-6-[cis-4-(N-methanesulphonyl-N-methyl-amino)-cyclohexan-1-yloxy]-7-methoxy-quinazoline, 4-[(3-chloro-4-fluoro-phenyl)amino]-6-[cis-4-(N-acetyl-N-methyl-amino)-cyclohexan-1-yloxy]-7-methoxy-quinazoline, 4-[(3-chloro-4-fluoro-phenyl)amino]-6-(trans-4-methylamino-cyclohexan-1-yloxy)-7-methoxy-quinazoline, 4-[(3-chloro-4-fluoro-phenyl)amino]-6-[trans-4-(N-methanesulphonyl-N-methyl-amino)-cyclohexan-1-yloxy]-7-methoxy-quinazoline, 4-[(3-chloro-4-fluoro-phenyl)amino]-6-(trans-4-dimethylamino-cyclohexan-1-yloxy)-7-methoxy-quinazoline, 4-[(3-chloro-4-fluoro-phenyl)amino]-6-(trans-4-{N-[(morpholin-4-yl)carbonyl]-N-methyl-amino}-cyclohexan-1-yloxy)-7-methoxy-quinazoline, 4-[(3-chloro-4-fluoro-phenyl)amino]-6-[2-(2,2-dimethyl-6-oxo-morpholin-4-yl)-ethoxy]-7-[(S)-(tetrahydrofuran-2-yl)methoxy]-quinazoline, 4-[(3-chloro-4-fluoro-phenyl)amino]-6-(1-methanesulphonyl-piperidin-4-yloxy)-7-methoxy-quinazoline, 4-[(3-chloro-4-fluoro-phenyl)amino]-6-(1-cyano-piperidin-4-yloxy)-7-methoxy-quinazoline, cetuximab, trastuzumab, ABX-EGF and Mab ICR-62, optionally in the form of the racemates, enantiomers or diastereomers thereof, optionally in the form of the pharmacologically acceptable acid addition salts thereof, the solvates and/or hydrates thereof.

In such medicament combinations the EGFR-inhibitor 2e is preferably selected from among 4-[(3-chloro-4-fluorophenyl)amino]-6-{[4-(morpholin-4-yl)-1-oxo-2-buten-1-yl]amino}-7-cyclopropylmethoxy-quinazoline, 4-[(3-chloro-4-fluorophenyl)amino]-6-{[4-(N,N-diethylamino)-1-oxo-2-buten-1-yl]amino}-7-cyclopropylmethoxy-quinazoline, 4-[(3-chloro-4-fluorophenyl)amino]-6-{[4-(N,N-dimethylamino)-1-oxo-2-buten-1-yl]amino}-7-cyclopropylmethoxy-quinazoline, 4-[(R)-(1-phenyl-ethyl)amino]-6-{[4-(morpholin-4-yl)-1-oxo-2-buten-1-yl]amino}-7-cyclopentyloxy-quinazoline, 4-[(3-chloro-4-fluoro-phenyl)amino]-6-{[4-((R)-6-methyl-2-oxo-morpholin-4-yl)-1-oxo-2-buten-1-yl]amino}-7-cyclopropylmethoxy-quinazoline, 4-[(3-chloro-4-fluoro-phenyl)amino]-6-{[4-((R)-6-methyl-2-oxo-morpholin-4-yl)-1-oxo-2-buten-1-yl]amino}-7-[(S)-(tetrahydrofuran-3-yl)oxy]-quinazoline, 4-[(3-chloro-4-fluoro-phenyl)amino]-6-{[4-((R)-2-methoxymethyl-6-oxo-morpholin-4-yl)-1-oxo-2-buten-1-yl]amino}-7-cyclopropylmethoxy-quinazoline, 4-[(3-chloro-4-fluoro-phenyl)amino]-6-[2-((S)-6-methyl-2-oxo-morpholin-4-yl)-ethoxy]-7-methoxy-quinazoline, 4-[(3-chloro-4-fluorophenyl)amino]-6-({4-[N-(2-methoxy-ethyl)-N-methyl-amino]-1-oxo-2-buten-1-yl}amino)-7-cyclopropylmethoxy-quinazoline, 4-[(3-chloro-4-fluorophenyl)amino]-6-{[4-(N,N-dimethylamino)-1-oxo-2-buten-1-yl]amino}-7-cyclopentyloxy-quinazoline, 4-[(R)-(1-phenyl-ethyl)amino]-6-{[4-(N,N-bis-(2-methoxy-ethyl)-amino)-1-oxo-2-buten-1-yl]amino}-7-cyclopropylmethoxy-quinazoline, 4-[(R)-(1-phenyl-ethyl)amino]-6-({4-[N-(2-methoxy-ethyl)-N-ethyl-amino]-1-oxo-2-buten-1-yl}amino)-7-cyclopropylmethoxy-quinazoline, 4-[(R)-(1-phenyl-ethyl)amino]-6-({4-[N-(2-methoxy-ethyl)-N-methyl-amino]-1-oxo-2-buten-1-yl}amino)-7-cyclopropylmethoxy-quinazoline, 4-[(R)-(1-phenyl-ethyl)amino]-6-({4-[N-(tetrahydropyran-4-yl)-N-methyl-amino]-1-oxo-2-buten-1-yl}amino)-7-cyclopropylmethoxy-quinazoline, 4-[(3-chloro-4-fluorophenyl)amino]-6-{[4-(N,N-dimethylamino)-1-oxo-2-buten-1-yl]amino}-7-((R)-tetrahydrofuran-3-yloxy)-quinazoline, 4-[(3-chloro-4-fluorophenyl)amino]-6-{[4-(N,N-dimethylamino)-1-oxo-2-buten-1-yl]amino}-7-((S)-tetrahydrofuran-3-yloxy)-quinazoline, 4-[(3-chloro-4-fluorophenyl)amino]-6-({4-[N-(2-methoxy-ethyl)-N-methyl-amino]-1-oxo-2-buten-1-yl}amino)-7-cyclopentyloxy-quinazoline, 4-[(3-chloro-4-fluorophenyl)amino]-6-{[4-(N-cyclopropyl-N-methyl-amino)-1-oxo-2-buten-1-yl]amino}-7-cyclopentyloxy-quinazoline, 4-[(3-chloro-4-fluorophenyl)amino]-6-{[4-(N,N-dimethylamino)-1-oxo-2-buten-1-yl]amino}-7-[(R)-(tetrahydrofuran-2-yl)methoxy]-quinazoline, 4-[(3-chloro-4-fluorophenyl)amino]-6-{[4-(N,N-dimethylamino)-1-oxo-2-buten-1-yl]amino}-7-[(S)-(tetrahydrofuran-2-yl)methoxy]-quinazoline, 4-[(3-ethynyl-phenyl)amino]-6,7-bis-(2-methoxy-ethoxy)-quinazoline, 4-[(3-chloro-4-fluorophenyl)amino]-7-[3-(morpholin-4-yl)-propyloxy]-6-[(vinyl-carbonyl)amino]-quinazoline, 4-[(R)-(1-phenyl-ethyl)amino]-6-(4-hydroxy-phenyl)-7H-pyrrolo[2,3-d]pyrimidine, 3-cyano-4-[(3-chloro-4-fluorophenyl)amino]-6-{[4-(N,N-dimethylamino)-1-oxo-2-buten-1-yl]amino}-7-ethoxy-quinoline, 4-{[3-chloro-4-(3-fluoro-benzyloxy)-phenyl]amino}-6-(5-{[(2-methanesulphonyl-ethyl)amino]methyl}-furan-2-yl)quinazoline, 4-[(R)-(1-phenyl-ethyl)amino]-6-{[4-((R)-6-methyl-2-oxo-morpholin-4-yl)-1-oxo-2-buten-1-yl]amino}-7-methoxy-quinazoline, 4-[(3-chloro-4-fluorophenyl)amino]-6-{[4-(morpholin-4-yl)-1-oxo-2-buten-1-yl]amino}-7-[(tetrahydrofuran-2-yl)methoxy]-quinazoline, 4-[(3-chloro-4-fluorophenyl)amino]-6-({4-[N,N-bis-(2-methoxy-ethyl)-amino]-1-oxo-2-buten-1-yl}amino)-7-[(tetrahydrofuran-2-yl)methoxy]-quinazoline, 4-[(3-ethynyl-phenyl)amino]-6-{[4-(5,5-dimethyl-2-oxo-morpholin-4-yl)-1-oxo-2-buten-1-yl]amino}-quinazoline, 4-[(3-chloro-4-fluoro-phenyl)amino]-6-[2-(2,2-dimethyl-6-oxo-morpholin-4-yl)-ethoxy]-7-methoxy-quinazoline, 4-[(3-chloro-4-fluoro-phenyl)amino]-6-[2-(2,2-dimethyl-6-oxo-morpholin-4-yl)-ethoxy]-7-[(R)-(tetrahydrofuran-2-yl)methoxy]-quinazoline, 4-[(3-chloro-4-fluoro-phenyl)amino]-7-[2-(2,2-dimethyl-6-oxo-morpholin-4-yl)-ethoxy]-6-[(S)-(tetrahydrofuran-2-yl)methoxy]-quinazoline, 4-[(3-chloro-4-fluoro-phenyl)amino]-6-{2-[4-(2-oxo-morpholin-4-yl)-piperidin-1-yl]-ethoxy}-7-methoxy-quinazoline, 4-[(3-chloro-4-fluoro-phenyl)amino]-6-[1-(tert.-butyloxycarbonyl)-piperidin-4-yloxy]-7-methoxy-quinazoline, 4-[(3-chloro-4-fluoro-phenyl)amino]-6-(trans-4-amino-cyclohexan-1-yloxy)-7-methoxy-quinazoline, 4-[(3-chloro-4-fluoro-phenyl)amino]-6-(trans-4-methanesulphonylamino-cyclohexan-1-yloxy)-7-methoxy-quinazoline, 4-[(3-chloro-4-fluoro-phenyl)amino]-6-(tetrahydropyran-3-yloxy)-7-methoxy-quinazoline, 4-[(3-chloro-4-fluoro-phenyl)amino]-6-(1-methyl-piperidin-4-yloxy)-7-methoxy-quinazoline, 4-[(3-chloro-4-fluoro-phenyl)amino]-6-{1-[(morpholin-4-yl)carbonyl]-piperidin-4-yloxy}-7-methoxy-quinazoline, 4-[(3-chloro-4-fluoro-phenyl)amino]-6-{1-[(methoxymethyl)carbonyl]-piperidin-4-yloxy}-7-methoxy-quinazoline, 4-[(3-chloro-4-fluoro-phenyl)amino]-6-(piperidin-3-yloxy)-7-methoxy-quinazoline, 4-[(3-chloro-4-fluoro-phenyl)amino]-6-[1-(2-acetylamino-ethyl)-piperidin-4-yloxy]-7-methoxy-quinazoline, 4-[(3-chloro-4-fluoro-phenyl)amino]-6-(tetrahydropyran-4-yloxy)-7-ethoxy-quinazoline, 4-[(3-chloro-4-fluoro-phenyl)amino]-6-((S)-tetrahydrofuran-3-yloxy)-7-hydroxy-quinazoline, 4-[(3-chloro-4-fluoro-phenyl)amino]-6-(tetrahydropyran-4-yloxy)-7-(2-methoxy-ethoxy)-quinazoline, 4-[(3-chloro-4-fluoro-phenyl)amino]-6-{trans-4-[(dimethylamino)sulphonylamino]-cyclohexan-1-yloxy}-7-methoxy-quinazoline, 4-[(3-chloro-4-fluoro-phenyl)amino]-6-{trans-4-[(morpholin-4-yl)carbonylamino]-cyclohexan-1-yloxy}-7-methoxy-quinazoline, 4-[(3-chloro-4-fluoro-phenyl)amino]-6-{trans-4-[(morpholin-4-yl)sulphonylamino]-cyclohexan-1-yloxy}-7-methoxy-quinazoline, 4-[(3-chloro-4-fluoro-phenyl)amino]-6-(tetrahydropyran-4-yloxy)-7-(2-acetylamino-ethoxy)-quinazoline, 4-[(3-chloro-4-fluoro-phenyl)amino]-6-(tetrahydropyran-4-yloxy)-7-(2-methanesulphonylamino-ethoxy)-quinazoline, 4-[(3-chloro-4-fluoro-phenyl)amino]-6-{1-[(piperidin-1-yl)carbonyl]-piperidin-4-yloxy}-7-methoxy-quinazoline, 4-[(3-chloro-4-fluoro-phenyl)amino]-6-(1-aminocarbonylmethyl-piperidin-4-yloxy)-7-methoxy-quinazoline, 4-[(3-chloro-4-fluoro-phenyl)amino]-6-(cis-4-{N-[(tetrahydropyran-4-yl)carbonyl]-N-methyl-amino}-cyclohexan-1-yloxy)-7-methoxy-quinazoline, 4-[(3-chloro-4-fluoro-phenyl)amino]-6-(cis-4-{N-[(morpholin-4-yl)carbonyl]-N-methyl-amino}-cyclohexan-1-yloxy)-7-methoxy-quinazoline, 4-[(3-chloro-4-fluoro-phenyl)amino]-6-(cis-4-{N-[(morpholin-4-yl)sulphonyl]-N-methyl-amino}-cyclohexan-1-yloxy)-7-methoxy-quinazoline, 4-[(3-chloro-4-fluoro-phenyl)amino]-6-(trans-4-ethanesulphonylamino-cyclohexan-1-yloxy)-7-methoxy-quinazoline, 4-[(3-chloro-4-fluoro-phenyl)amino]-6-(1-methanesulphonyl-piperidin-4-yloxy)-7-ethoxy-quinazoline, 4-[(3-chloro-4-fluoro-phenyl)amino]-6-(1-methanesulphonyl-piperidin-4-yloxy)-7-(2-methoxy-ethoxy)-quinazoline, 4-[(3-chloro-4-fluoro-phenyl)amino]-6-[1-(2-methoxy-acetyl)-piperidin-4-yloxy]-7-(2-methoxy-ethoxy)-quinazoline, 4-[(3-chloro-4-fluoro-phenyl)amino]-6-(cis-4-acetylamino-cyclohexan-1-yloxy)-7-methoxy-quinazoline, 4-[(3-ethynyl-phenyl)amino]-6-[1-(tert.-butyloxycarbonyl)-piperidin-4-yloxy]-7-methoxy-quinazoline, 4-[(3-ethynyl-phenyl)amino]-6-(tetrahydropyran-4-yloxy]-7-methoxy-quinazoline, 4-[(3-chloro-4-fluoro-phenyl)amino]-6-(cis-4-{N-[(piperidin-1-yl)carbonyl]-N-methyl-amino}-cyclohexan-1-yloxy)-7-methoxy-quinazoline, 4-[(3-chloro-4-fluoro-phenyl)amino]-6-(cis-4-{N-[(4-methyl-piperazin-1-yl)carbonyl]-N-methyl-amino}-cyclohexan-1-yloxy)-7-methoxy-quinazoline, 4-[(3-chloro-4-fluoro-phenyl)amino]-6-{cis-4-[(morpholin-4-yl)carbonylamino]-cyclohexan-1-yloxy}-7-methoxy-quinazoline, 4-[(3-chloro-4-fluoro-phenyl)amino]-6-{1-[2-(2-oxopyrrolidin-1-yl)ethyl]-piperidin-4-yloxy}-7-methoxy-quinazoline, 4-[(3-chloro-4-fluoro-phenyl)amino]-6-{1-[(morpholin-4-yl)carbonyl]-piperidin-4-yloxy}-7-(2-methoxy-ethoxy)-quinazoline, 4-[(3-ethynyl-phenyl)amino]-6-(1-acetyl-piperidin-4-yloxy)-7-methoxy-quinazoline, 4-[(3-ethynyl-phenyl)amino]-6-(1-methyl-piperidin-4-yloxy)-7-methoxy-quinazoline, 4-[(3-ethynyl-phenyl)amino]-6-(1-methanesulphonyl-piperidin-4-yloxy)-7-methoxy-quinazoline, 4-[(3-chloro-4-fluoro-phenyl)amino]-6-(1-methyl-piperidin-4-yloxy)-7(2-methoxy-ethoxy)-quinazoline, 4-[(3-chloro-4-fluoro-phenyl)amino]-6-(1-isopropyloxycarbonyl-piperidin-4-yloxy)-7-methoxy-quinazoline, 4-[(3-chloro-4-fluoro-phenyl)amino]-6-(cis-4-methylamino-cyclohexan-1-yloxy)-7-methoxy-quinazoline, 4-[(3-chloro-4-fluoro-phenyl)amino]-6-{cis-4-[N-(2-methoxy-acetyl)-N-methyl-amino]-cyclohexan-1-yloxy}-7-methoxy-quinazoline, 4-[(3-ethynyl-phenyl)amino]-6-(piperidin-4-yloxy)-7-methoxy-quinazoline, 4-[(3-ethynyl-phenyl)amino]-6-[1-(2-methoxy-acetyl)-piperidin-4-yloxy]-7-methoxy-quinazoline, 4-[(3-ethynyl-phenyl)amino]-6-{1-[(morpholin-4-yl)carbonyl]-piperidin-4-yloxy}-7-methoxy-quinazoline, 4-[(3-chloro-4-fluoro-phenyl)amino]-6-{1-[(cis-2.6-dimethyl-morpholin-4-yl)carbonyl]-piperidin-4-yloxy}-7-methoxy-quinazoline, 4-[(3-chloro-4-fluoro-phenyl)amino]-6-{1-[(2-methyl-morpholin-4-yl)carbonyl]-piperidin-4-yloxy}-7-methoxy-quinazoline, 4-[(3-chloro-4-fluoro-phenyl)amino]-6-{1-[(S,S)-(2-oxa-5-aza-bicyclo[2,2,1]hept-5-yl)carbonyl]-piperidin-4-yloxy}-7-methoxy-quinazoline, 4-[(3-chloro-4-fluoro-phenyl)amino]-6-{1-[(N-methyl-N-2-methoxyethyl-amino)carbonyl]-piperidin-4-yloxy}-7-methoxy-quinazoline, 4-[(3-chloro-4-fluoro-phenyl)amino]-6-(1-ethyl-piperidin-4-yloxy)-7-methoxy-quinazoline, 4-[(3-chloro-4-fluoro-phenyl)amino]-6-{1-[(2-methoxyethyl)carbonyl]-piperidin-4-yloxy}-7-methoxy-quinazoline, 4-[(3-chloro-4-fluoro-phenyl)amino]-6-{1-[(3-methoxypropyl-amino)-carbonyl]-piperidin-4-yloxy}-7-methoxy-quinazoline, 4-[(3-chloro-4-fluoro-phenyl)amino]-6-[cis-4-(N-methanesulphonyl-N-methyl-amino)-cyclohexan-1-yloxy]-7-methoxy-quinazoline, 4-[(3-chloro-4-fluoro-phenyl)amino]-6-[cis-4-(N-acetyl-N-methyl-amino)-cyclohexan-1-yloxy]-7-methoxy-quinazoline, 4-[(3-chloro-4-fluoro-phenyl)amino]-6-(trans-4-methylamino-cyclohexan-1-yloxy)-7-methoxy-quinazoline, 4-[(3-chloro-4-fluoro-phenyl)amino]-6-[trans-4-(N-methanesulphonyl-N-methyl-amino)-cyclohexan-1-yloxy]-7-methoxy-quinazoline, 4-[(3-chloro-4-fluoro-phenyl)amino]-6-(trans-4-dimethylamino-cyclohexan-1-yloxy)-7-methoxy-quinazoline, 4-[(3-chloro-4-fluoro-phenyl)amino]-6-(trans-4-{N-[(morpholin-4-yl)carbonyl]-N-methyl-amino}-cyclohexan-1-yloxy)-7-methoxy-quinazoline, 4-[(3-chloro-4-fluoro-phenyl)amino]-6-[2-(2,2-dimethyl-6-oxo-morpholin-4-yl)-ethoxy]-7-[(S)-(tetrahydrofuran-2-yl)methoxy]-quinazoline, 4-[(3-chloro-4-fluoro-phenyl)amino]-6-(1-methanesulphonyl-piperidin-4-yloxy)-7-methoxy-quinazoline, 4-[(3-chloro-4-fluoro-phenyl)amino]-6-(1-cyano-piperidin-4-yloxy)-7-methoxy-quinazoline, and cetuximab, optionally in the form of the racemates, enantiomers or diastereomers thereof, optionally in the form of the pharmacologically acceptable acid addition salts thereof, the solvates and/or hydrates thereof.

Particularly preferably, the EGFR-inhibitors 2a used within the scope of the medicament combinations according to the invention are selected from the group comprising 4-[(3-chloro-4-fluorophenyl)amino]-6-{[4-(morpholin-4-yl)-1-oxo-2-buten-1-yl]amino}-7-cyclopropylmethoxy-quinazoline, 4-[(R)-(1-phenyl-ethyl)amino]-6-{[4-(morpholin-4-yl)-1-oxo-2-buten-1-yl]amino}-7-cyclopentyloxy-quinazoline, 4-[(3-chloro-4-fluoro-phenyl)amino]-6-{[4-((R)-6-methyl-2-oxo-morpholin-4-yl)-1-oxo-2-buten-1-yl]amino}-7-[(S)-(tetrahydrofuran-3-yl)oxy]-quinazoline, 4-[(3-chloro-4-fluoro-phenyl)amino]-6-[2-((S)-6-methyl-2-oxo-morpholin-4-yl)-ethoxy]-7-methoxy-quinazoline, 4-[(3-chloro-4-fluorophenyl)amino]-6-({4-[N-(2-methoxy-ethyl)-N-methyl-amino]-1-oxo-2-buten-1-yl}amino)-7-cyclopropylmethoxy-quinazoline, 4-[(R)-(1-phenyl-ethyl)amino]-6-({4-[N-(tetrahydropyran-4-yl)-N-methyl-amino]-1-oxo-2-buten-1-yl}amino)-7-cyclopropylmethoxy-quinazoline, 4-[(3-chloro-4-fluorophenyl)amino]-6-({4-[N-(2-methoxy-ethyl)-N-methyl-amino]-1-oxo-2-buten-1-yl}amino)-7-cyclopentyloxy-quinazoline, 4-[(3-chloro-4-fluorophenyl)amino]-6-{[4-(N,N-dimethylamino)-1-oxo-2-buten-1-yl]amino}-7-[(R)-(tetrahydrofuran-2-yl)methoxy]-quinazoline, 4-[(3-ethynyl-phenyl)amino]-6,7-bis-(2-methoxy-ethoxy)-quinazoline, 4-[(R)-(1-phenyl-ethyl)amino]-6-(4-hydroxy-phenyl)-7H-pyrrolo[2,3-d]pyrimidine, 3-cyano-4-[(3-chloro-4-fluorophenyl)amino]-6-{[4-(N,N-dimethylamino)-1-oxo-2-buten-1-yl]amino}-7-ethoxy-quinoline, 4-[(R)-(1-phenyl-ethyl)amino]-6-{[4-((R)-6-methyl-2-oxo-morpholin-4-yl)-1-oxo-2-buten-1-yl]amino}-7-methoxy-quinazoline, 4-[(3-chloro-4-fluorophenyl)amino]-6-{[4-(morpholin-4-yl)-1-oxo-2-buten-1-yl]amino}-7-[(tetrahydrofuran-2-yl)methoxy]-quinazoline, 4-[(3-ethynyl-phenyl)amino]-6-{[4-(5,5-dimethyl-2-oxo-morpholin-4-yl)-1-oxo-2-buten-1-yl]amino}-quinazoline, 4-[(3-chloro-4-fluoro-phenyl)amino]-6-{2-[4-(2-oxo-morpholin-4-yl)-piperidin-1-yl]-ethoxy}-7-methoxy-quinazoline, 4-[(3-chloro-4-fluoro-phenyl)amino]-6-(trans-4-amino-cyclohexan-1-yloxy)-7-methoxy-quinazoline, 4-[(3-chloro-4-fluoro-phenyl)amino]-6-(trans-4-methanesulphonylamino-cyclohexan-1-yloxy)-7-methoxy-quinazoline, 4-[(3-chloro-4-fluoro-phenyl)amino]-6-(tetrahydropyran-3-yloxy)-7-methoxy-quinazoline, 4-[(3-chloro-4-fluoro-phenyl)amino]-6-{1-[(morpholin-4-yl)carbonyl]-piperidin-4-yloxy}-7-methoxy-quinazoline, 4-[(3-chloro-4-fluoro-phenyl)amino]-6-(piperidin-3-yloxy)-7-methoxy-quinazoline, 4-[(3-chloro-4-fluoro-phenyl)amino]-6-[1-(2-acetylamino-ethyl)-piperidin-4-yloxy]-7-methoxy-quinazoline, 4-[(3-chloro-4-fluoro-phenyl)amino]-6-(tetrahydropyran-4-yloxy)-7-ethoxy-quinazoline, 4-[(3-chloro-4-fluoro-phenyl)amino]-6-{trans-4-[(morpholin-4-yl)carbonylamino]-cyclohexan-1-yloxy}-7-methoxy-quinazoline, 4-[(3-chloro-4-fluoro-phenyl)amino]-6-{1-[(piperidin-1-yl)carbonyl]-piperidin-4-yloxy}-7-methoxy-quinazoline, 4-[(3-chloro-4-fluoro-phenyl)amino]-6-(cis-4-{N-[(morpholin-4-yl)carbonyl]-N-methyl-amino}-cyclohexan-1-yloxy)-7-methoxy-quinazoline, 4-[(3-chloro-4-fluoro-phenyl)amino]-6-(trans-4-ethanesulphonylamino-cyclohexan-1-yloxy)-7-methoxy-quinazoline, 4-[(3-chloro-4-fluoro-phenyl)amino]-6-(1-methanesulphonyl-piperidin-4-yloxy)-7-(2-methoxy-ethoxy)-quinazoline, 4-[(3-chloro-4-fluoro-phenyl)amino]-6-[1-(2-methoxy-acetyl)-piperidin-4-yloxy]-7-(2-methoxy-ethoxy)-quinazoline, 4-[(3-ethynyl-phenyl)amino]-6-(tetrahydropyran-4-yloxy]-7-methoxy-quinazoline, 4-[(3-chloro-4-fluoro-phenyl)amino]-6-(cis-4-{N-[(piperidin-1-yl)carbonyl]-N-methyl-amino}-cyclohexan-1-yloxy)-7-methoxy-quinazoline, 4-[(3-chloro-4-fluoro-phenyl)amino]-6-{cis-4-[(morpholin-4-yl)carbonylamino]-cyclohexan-1-yloxy}-7-methoxy-quinazoline, 4-[(3-chloro-4-fluoro-phenyl)amino]-6-{1-[2-(2-oxopyrrolidin-1-yl)ethyl]-piperidin-4-yloxy}-7-methoxy-quinazoline, 4-[(3-ethynyl-phenyl)amino]-6-(1-acetyl-piperidin-4-yloxy)-7-methoxy-quinazoline, 4-[(3-ethynyl-phenyl)amino]-6-(1-methyl-piperidin-4-yloxy)-7-methoxy-quinazoline, 4-[(3-ethynyl-phenyl)amino]-6-(1-methanesulphonyl-piperidin-4-yloxy)-7-methoxy-quinazoline, 4-[(3-chloro-4-fluoro-phenyl)amino]-6-(1-methyl-piperidin-4-yloxy)-7(2-methoxy-ethoxy)-quinazoline, 4-[(3-ethynyl-phenyl)amino]-6-{1-[(morpholin-4-yl)carbonyl]-piperidin-4-yloxy}-7-methoxy-quinazoline, 4-[(3-chloro-4-fluoro-phenyl)amino]-6-{1-[(N-methyl-N-2-methoxyethyl-amino)carbonyl]-piperidin-4-yloxy}-7-methoxy-quinazoline, 4-[(3-chloro-4-fluoro-phenyl)amino]-6-(1-ethyl-piperidin-4-yloxy)-7-methoxy-quinazoline, 4-[(3-chloro-4-fluoro-phenyl)amino]-6-[cis-4-(N-methanesulphonyl-N-methyl-amino)-cyclohexan-1-yloxy]-7-methoxy-quinazoline, 4-[(3-chloro-4-fluoro-phenyl)amino]-6-[cis-4-(N-acetyl-N-methyl-amino)-cyclohexan-1-yloxy]-7-methoxy-quinazoline, 4-[(3-chloro-4-fluoro-phenyl)amino]-6-(trans-4-methylamino-cyclohexan-1-yloxy)-7-methoxy-quinazoline, 4-[(3-chloro-4-fluoro-phenyl)amino]-6-[trans-4-(N-methanesulphonyl-N-methyl-amino)-cyclohexan-1-yloxy]-7-methoxy-quinazoline, 4-[(3-chloro-4-fluoro-phenyl)amino]-6-(trans-4-dimethylamino-cyclohexan-1-yloxy)-7-methoxy-quinazoline, 4-[(3-chloro-4-fluoro-phenyl)amino]-6-(trans-4-{N-[(morpholin-4-yl)carbonyl]-N-methyl-amino}-cyclohexan-1-yloxy)-7-methoxy-quinazoline, 4-[(3-chloro-4-fluoro-phenyl)amino]-6-[2-(2,2-dimethyl-6-oxo-morpholin-4-yl)-ethoxy]-7-[(S)-(tetrahydrofuran-2-yl)methoxy]-quinazoline, 4-[(3-chloro-4-fluoro-phenyl)amino]-6-(1-methanesulphonyl-piperidin-4-yloxy)-7-methoxy-quinazoline, 4-[(3-chloro-4-fluoro-phenyl)amino]-6-(1-cyano-piperidin-4-yloxy)-7-methoxy-quinazoline, and 4-[(3-chloro-4-fluoro-phenyl)amino]-6-{1-[(2-methoxyethyl)carbonyl]-piperidin-4-yloxy}-7-methoxy-quinazoline, optionally in the form of the racemates, enantiomers or diastereomers thereof, optionally in the form of the pharmacologically acceptable acid addition salts thereof, the solvates and/or hydrates thereof.

Particularly preferred medicament combinations according to the invention contain as EGFR-inhibitors 2e those compounds which are selected from the group comprising
4-[(3-chloro-4-fluorophenyl)amino]-6-{[4-(morpholin-4-yl)-1-oxo-2-buten-1-yl]-amino}-7-cyclopropylmethoxy-quinazoline (2e.1),
4-[(3-chloro-4-fluoro-phenyl)amino]-6-{[4-((R)-6-methyl-2-oxo-morpholin-4-yl)-1-oxo-2-buten-1-yl]amino}-7-[(S)-(tetrahydrofuran-3-yl)oxy]-quinazoline (2e.2),
4-[(3-chloro-4-fluoro-phenyl)amino]-6-[2-((S)-6-methyl-2-oxo-morpholin-4-yl)-ethoxy]-7-methoxy-quinazoline (2e.3),
4-[(3-chloro-4-fluorophenyl)amino]-6-({4-[N-(2-methoxy-ethyl)-N-methyl-amino]-1-oxo-2-buten-1-yl}amino)-7-cyclopropylmethoxy-quinazoline (2e.4),
4-[(3-ethynyl-phenyl)amino]-6,7-bis-(2-methoxy-ethoxy)-quinazoline (2e.5),
4-[(3-chloro-4-fluorophenyl)amino]-6-{[4-(morpholin-4-yl)-1-oxo-2-buten-1-yl]-amino}-7-[(tetrahydrofuran-2-yl)methoxy]-quinazoline (2e 0.6),
4-[(3-ethynyl-phenyl)amino]-6-{[4-(5,5-dimethyl-2-oxo-morpholin-4-yl)-1-oxo-2-buten-1-yl]amino}-quinazoline (2e.7),
4-[(3-chloro-4-fluoro-phenyl)amino]-6-(trans-4-methanesulphonylamino-cyclohexan-1-yloxy)-7-methoxy-quinazoline (2e.8),
4-[(3-chloro-4-fluoro-phenyl)amino]-6-(tetrahydropyran-3-yloxy)-7-methoxy-quinazoline (2e.9),
4-[(3-chloro-4-fluoro-phenyl)amino]-6-{1-[(morpholin-4-yl)carbonyl]-piperidin-4-yloxy}-7-methoxy-quinazoline (2e.10),
4-[(3-chloro-4-fluoro-phenyl)amino]-6-{1-[2-(2-oxopyrrolidin-1-yl)ethyl]-piperidin-4-yloxy}-7-methoxy-quinazoline (2e.11),
4-[(3-ethynyl-phenyl)amino]-6-(1-acetyl-piperidin-4-yloxy)-7-methoxy-quinazoline (2e.12),
4-[(3-ethynyl-phenyl)amino]-6-(1-methyl-piperidin-4-yloxy)-7-methoxy-quinazoline (2e.13),
4-[(3-ethynyl-phenyl)amino]-6-(1-methanesulphonyl-piperidin-4-yloxy)-7-methoxy-quinazoline (2e.14),
4-[(3-ethynyl-phenyl)amino]-6-{1-[(morpholin-4-yl)carbonyl]-piperidin-4-yloxy}-7-methoxy-quinazoline (2e.15),
4-[(3-chloro-4-fluoro-phenyl)amino]-6-{1-[(2-methoxyethyl)carbonyl]-piperidin-4-yloxy}-7-methoxy-quinazoline (2e.16),
4-[(3-chloro-4-fluoro-phenyl)amino]-6-[cis-4-(N-methanesulphonyl-N-methyl-amino)-cyclohexan-1-yloxy]-7-methoxy-quinazoline (2e.17),
4-[(3-chloro-4-fluoro-phenyl)amino]-6-[cis-4-(N-acetyl-N-methyl-amino)-cyclohexan-1-yloxy]-7-methoxy-quinazoline (2e.18),
4-[(3-chloro-4-fluoro-phenyl)amino]-6-(trans-4-methylamino-cyclohexan-1-yloxy)-7-methoxy-quinazoline (2e.19),
4-[(3-chloro-4-fluoro-phenyl)amino]-6-[trans-4-(N-methanesulphonyl-N-methyl-amino)-cyclohexan-1-yloxy]-7-methoxy-quinazoline (2e.20),
4-[(3-chloro-4-fluoro-phenyl)amino]-6-(trans-4-dimethylamino-cyclohexan-1-yloxy)-7-methoxy-quinazoline (2e.21),
4-[(3-chloro-4-fluoro-phenyl)amino]-6-(trans-4-{N-[(morpholin-4-yl)carbonyl]-N-methyl-amino}-cyclohexan-1-yloxy)-7-methoxy-quinazoline (2e.22),
4-[(3-chloro-4-fluoro-phenyl)amino]-6-[2-(2,2-dimethyl-6-oxo-morpholin-4-yl)-ethoxy]-7-[(S)-(tetrahydrofuran-2-yl)methoxy]-quinazoline (2e.23),
4-[(3-chloro-4-fluoro-phenyl)amino]-6-(1-methanesulphonyl-piperidin-4-yloxy)-7-methoxy-quinazoline (2e.24) and
4-[(3-chloro-4-fluoro-phenyl)amino]-6-(1-cyano-piperidin-4-yloxy)-7-methoxy-quinazoline (2e.25),
   optionally in the form of the racemates, enantiomers or diastereomers thereof, optionally in the form of the pharmacologically acceptable acid addition salts thereof, the solvates and/or hydrates thereof.

By the acid addition salts with pharmacologically acceptable acids which the compounds 2e may possibly be capable of forming are meant for example salts selected from the group comprising the hydrochloride, hydrobromide, hydriodide, hydrosulphate, hydrophosphate, hydromethanesulphonate, hydronitrate, hydromaleate, hydroacetate, hydrobenzoate, hydrocitrate, hydrofumarate, hydrotartrate, hydroxalate, hydrosuccinate, hydrobenzoate and hydro-p-toluenesulphonate, preferably the hydrochloride, hydrobromide, hydrosulphate, hydrophosphate, hydrofumarate and hydromethanesulphonate.

Examples of novel preferred pharmaceutical combinations of preferred compounds of formula 1 with the above-mentioned EGFR-inhibitors 2e are combinations containing the compounds 1.4 and 2e.1; 1.4 and 2e.2; 1.4 and 2e.3; 1.4 and 2e.4; 1.4 and 2e.5; 1.4 and 2e.6; 1.4 and 2e.7; 1.4 and 2e.8; 1.4 and 2e.9; 1.4 and 2e.10; 1.4 and 2e.11; 1.4 and 2e.12; 1.4 and 2e.13; 1.4 and 2e.14; 1.4 and 2e.15; 1.4 and 2e.16; 1.4 and 2e.17; 1.4 and 2e.18; 1.4 and 2e.19; 1.4 and 2e.20; 1.4 and 2e.21; 1.4 and 2e.22; 1.4 and 2e.23; 1.4 and 2e.24; 1.4 and 2e.25; 1.5 and 2e.1; 1.5 and 2e.2; 1.5 and 2e.3; 1.5 and 2e.4; 1.5 and 2e.5; 1.5 and 2e.6; 1.5 and 2e.7; 1.5 and 2e.8; 1.5 and 2e.9; 1.5 and 2e.10; 1.5 and 2e.11; 1.5 and 2e.12; 1.5 and 2e.13; 1.5 and 2e.14; 1.5 and 2e.15; 1.5 and 2e.16; 1.5 and 2e.17; 1.5 and 2e.18; 1.5 and 2e.19; 1.5 and 2e.20; 1.5 and 2e.21; 1.5 and 2e.22; 1.5 and 2e.23; 1.5 and 2e.24; 1.5 and 2e.25; 1.6 and 2e.1; 1.6 and 2e.2; 1.6 and 2e.3; 1.6 and 2e.4; 1.6 and 2e.5; 1.6 and 2e.6; 1.6 and 2e.7; 1.6 and 2e.8; 1.6 and 2e.9; 1.6 and 2e.10; 1.6 and 2e.11; 1.6 and 2e.12; 1.6 and 2e.13; 1.6 and 2e.14; 1.6 and 2e.15; 1.6 and 2e.16; 1.6 and 2e.17; 1.6 and 2e.18; 1.6 and 2e.19; 1.6 and 2e.20; 1.6 and 2e.21; 1.6 and 2e.22; 1.6 and 2e.23; 1.6 and 2e.24; 1.6 and 2e.25; 1.7 and 2e.1; 1.7 and 2e.2; 1.7 and 2e.3; 1.7 and 2e.4; 1.7 and 2e.5; 1.7 and 2e.6; 1.7 and 2e.7; 1.7 and 2e.8; 1.7 and 2e.9; 1.7 and 2e.10; 1.7 and 2e.11; 1.7 and 2e.12; 1.7 and 2e.13; 1.7 and 2e.14; 1.7 and 2e.15; 1.7 and 2e.16; 1.7 and 2e.17; 1.7 and 2e.18; 1.7 and 2e.19; 1.7 and 2e.20; 1.7 and 2e.21; 1.7 and 2e.22; 1.7 and 2e.23; 1.7 and 2e.24; 1.7 and 2e.25; 1.8 and 2e.1; 1.8 and 2e.2; 1.8 and 2e.3; 1.8 and 2e.4; 1.8 and 2e.5; 1.8 and 2e.6; 1.8 and 2e.7; 1.8 and 2e.8; 1.8 and 2e.9; 1.8 and 2e.10; 1.8 and 2e.11; 1.8 and 2e.12; 1.8 and 2e.13; 1.8 and 2e.14; 1.8 and 2e.15; 1.8 and 2e.16; 1.8 and 2e.17; 1.8 and 2e.18; 1.8 and 2e.19; 1.8 and 2e.20; 1.8 and 2e.21; 1.8 and 2e.22; 1.8 and 2e.23; 1.8 and 2e.24; 1.8 and 2e.25; 1.9 and 2e.1; 1.9 and 2e.2; 1.9 and 2e.3; 1.9 and 2e.4; 1.9 and 2e.5; 1.9 and 2e.6; 1.9 and 2e.7; 1.9 and 2e.8; 1.9 and 2e.9; 1.9 and 2e.10; 1.9 and 2e.11; 1.9 and 2e.12; 1.9 and 2e.13; 1.9 and 2e.14; 1.9 and 2e.15; 1.9 and 2e.16; 1.9 and 2e.17; 1.9 and 2e.18; 1.9 and 2e.19; 1.9 and 2e.20; 1.9 and 2e.21; 1.9 and 2e.22; 1.9 and 2e.23; 1.9 and 2e.24; 1.9 and 2e.25; 1.10 and 2e.1; 1.10 and 2e.2; 1.10 and 2e.3; 1.10 and 2e.4; 1.10 and 2e.5; 1.10 and 2e.6; 1.10 and 2e.7; 1.10 and 2e.8; 1.10 and 2e.9; 1.10 and 2e.10; 1.10 and 2e.11; 1.10 and 2e.12; 1.10 and 2e.13; 1.10 and 2e.14; 1.10 and 2e.15; 1.10 and 2e.16; 1.10 and 2e.17; 1.10 and 2e.18; 1.10 and 2e.19; 1.10 and 2e.20; 1.10 and 2e.21; 1.10 and 2e.22; 1.10 and 2e.23; 1.10 and 2e.24; 1.10 and 2e.25; 1.11 and 2e.1; 1.11 and 2e.2; 1.11 and 2e.3; 1.11 and 2e.4; 1.11 and 2e.5; 1.11 and 2e.6; 1.11 and 2e.7; 1.11 and 2e.8; 1.11 and 2e.9; 1.11 and 2e.10; 1.11 and 2e.11; 1.11 and 2e.12; 1.11 and 2e.13; 1.11 and 2e.14; 1.11 and 2e.15; 1.11 and 2e.16; 1.11 and 2e.17; 1.11 and 2e.18; 1.11 and 2e.19; 1.11 and 2e.20; 1.11 and 2e.21; 1.11 and 2e.22; 1.11 and 2e.23; 1.11 and 2e.24; 1.11 and 2e.25; 1.12 and 2e.1; 1.12 and 2e.2; 1.12 and 2e.3; 1.12 and 2e.4; 1.12 and 2e.5; 1.12 and 2e.6; 1.12 and 2e.7; 1.12 and 2e.8; 1.12 and 2e.9; 1.12 and 2e.10; 1.12 and 2e.11; 1.12 and 2e.12; 1.12 and 2e.13; 1.12 and 2e.14; 1.12 and 2e.15; 1.12 and 2e.16; 1.12 and 2e.17; 1.12 and 2e.18; 1.12 and 2e.19; 1.12 and 2e.20; 1.12 and 2e.21; 1.12 and 2e.22; 1.12 and 2e.23; 1.12 and 2e.24; 1.12 and 2e.25; 1.13 and 2e.1; 1.13 and 2e.2; 1.13 and 2e.3; 1.13 and 2e.4; 1.13 and 2e.5; 1.13 and 2e.6; 1.13 and 2e.7; 1.13 and 2e.8; 1.13 and 2e.9; 1.13 and 2e.10; 1.13 and 2e.11; 1.13 and 2e.12; 1.13 and 2e.13; 1.13 and 2e.14; 1.13 and 2e.15; 1.13 and 2e.16; 1.13 and 2e.17; 1.13 and 2e.18; 1.13 and 2e.19; 1.13 and 2e.20; 1.13 and 2e.21; 1.13 and 2e.22; 1.13 and 2e.23; 1.13 and 2e.24; 1.13 and 2e.25; 1.14 and 2e.1; 1.14 and 2e.2; 1.14 and 2e.3; 1.14 and 2e.4; 1.14 and 2e.5; 1.14 and 2e.6; 1.14 and 2e.7; 1.14 and 2e.8; 1.14 and 2e.9; 1.14 and 2e.10; 1.14 and 2e.11; 1.14 and 2e.12; 1.14 and 2e.13; 1.14 and 2e.14; 1.14 and 2e.15; 1.14 and 2e.16; 1.14 and 2e.17; 1.14 and 2e.18; 1.14 and 2e.19; 1.14 and 2e.20; 1.14 and 2e.21; 1.14 and 2e.22; 1.14 and 2e.23; 1.14 and 2e.24; 1.14 and 2e.25; in each case optionally in the form of the racemates, enantiomers or diastereomers thereof and optionally in the form of the pharmacologically acceptable acid addition salts, solvates and/or hydrates thereof.

Of the above-mentioned combinations the preferred ones according to the invention are those that contain as compound of formula 1 one of the compounds 1.7, 1.8, 1.9, 1.10, 1.11, 1.12, 1.13, or 1.14. Of the above-mentioned combinations the preferred ones according to the invention are also those which contain as compound 2e one of the compounds 2e.1, 2e.2, 2e.3, 2e.4, 2e.10, 2e.11, 2e.14, 2e.16, 2e.17, 2e.18, 2e.19, 2e.20, 2e.21, 2e.22, 2e.23, 2e.24 or 2e.25, while those combinations that contain one of the compounds 2e.2, 2e.3 or 2e.4 are of particular importance according to the invention.

The novel medicament combinations comprising compounds of formula 1 with at least one other active substance 2 are not restricted to binary combinations of active substances. The combinations mentioned above, partly by way of example, which contain in addition to a compound of formula 1 one other active substance 2, may also contain a third or fourth, preferably a third active substance, which is also selected from the above-mentioned group of anticholinergics (2a), PDE IV inhibitors (2b), steroids (2c), LTD4-antagonists (2d) and EGFR-inhibitors (2e).

Particularly preferred combinations which contain two other active substances in addition to a compound of formula 1 are selected from the active substance combinations listed below. These are medicament combinations which may contain, for example:

A) a compound of formula 1, an anticholinergic (2a), a PDE IV inhibitor (2b);

B) a compound of formula 1, an anticholinergic (2a), a steroid (2c);

C) a compound of formula 1, an anticholinergic (2a), an LTD4 antagonist (2d);

D) a compound of formula 1, an anticholinergic (2a), an EGFR inhibitor (2e);

E) a compound of formula 1, a PDE-IV inhibitor (2b), a steroid (2c);

F) a compound of formula 1, a PDE-IV inhibitor (2b), an LTD4 antagonist (2d);

G) a compound of formula 1, a PDE-IV inhibitor (2b), an EGFR inhibitor (2e);

H) a compound of formula 1, a steroid (2c), an LTD4 antagonist (2d);

I) a compound of formula 1, a steroid (2c), an EGFR inhibitor (2e);

J) a compound of formula 1, an LTD4 antagonist (2d), an EGFR inhibitor (2e).

Of outstanding importance according to the invention are all those medicament combinations disclosed within the scope of the present invention which contain the compounds of formula 1 in the form of the R-enantiomers thereof.

### TERMS AND DEFINITIONS USED

Within the scope of the present invention, by a medicament combination of components 1 and 2 is meant the joint administration of both active substances in a single preparation or formulation or the separate administration of the two active substances in separate formulations. If the active substances 1 and 2 are administered in separate formulations, this separate administration may be done simultaneously or at different times, i.e. successively. In one aspect the present invention relates to the above-mentioned medicament combinations which contain in addition to therapeutically effective amounts of 1 and 2 a pharmaceutically acceptable carrier. In one aspect the present invention relates to the above-mentioned pharmaceutical compositions which do not contain a pharmaceutically acceptable carrier in addition to therapeutically effective amounts of 1 and 2.

The alkyl groups used, unless otherwise stated, are branched and unbranched alkyl groups having 1 to 4 carbon atoms. Examples include: methyl, ethyl, propyl or butyl. The groups methyl, ethyl, propyl or butyl may optionally also be referred to by the abbreviations Me, Et, Prop or Bu. Unless otherwise stated, the definitions propyl and butyl also include all possible isomeric forms of the groups in question. Thus, for example, propyl includes n-propyl and iso-propyl, butyl includes iso-butyl, sec. butyl and tert.-butyl, etc.

The cycloalkyl groups used, unless otherwise stated, are alicyclic groups with 3 to 6 carbon atoms. These are the cyclopropyl, cyclobutyl, cyclopentyl and cyclohexyl groups. According to the invention cyclopropyl is of particular importance within the scope of the present invention.

The alkylene groups used, unless otherwise stated, are branched and unbranched double-bonded alkyl bridges with 1 to 4 carbon atoms. Examples include: methylene, ethylene, propylene or butylene.

The alkylene-halogen groups used, unless otherwise stated, are branched and unbranched double-bonded alkyl bridges with 1 to 4 carbon atoms which may be mono-, di- or trisubstituted, preferably disubstituted, by a halogen. Accordingly, unless otherwise stated, the term alkylene-OH groups denotes branched and unbranched double-bonded alkyl bridges with 1 to 4 carbon atoms which may be mono-, di- or trisubstituted, preferably monosubstituted, by a hydroxy.

The alkyloxy groups used, unless otherwise stated, are branched and unbranched alkyl groups with 1 to 4 carbon atoms which are linked via an oxygen atom. The following may be mentioned, for example: methyloxy, ethyloxy, propyloxy or butyloxy. The groups methyloxy, ethyloxy, propyloxy or butyloxy may optionally also be referred to by the abbreviations MeO, EtO, PropO or BuO. Unless otherwise stated, the definitions propyloxy and butyloxy also include all possible isomeric forms of the groups in question. Thus, for example, propyloxy includes n-propyloxy and iso-propyloxy, butyloxy includes iso-butyloxy, sec. butyloxy and tert.-butyloxy, etc. The word alkoxy may also possibly be used within the scope of the present invention instead of the word alkyloxy. The groups methyloxy, ethyloxy, propyloxy or butyloxy may optionally also be referred to as methoxy, ethoxy, propoxy or butoxy.

The alkylene-alkyloxy groups used, unless otherwise stated, are branched and unbranched double-bonded alkyl bridges with 1 to 4 carbon atoms which may be mono-, di- or trisubstituted, preferably monosubstituted, by an alkyloxy group.

The —O—CO-alkyl groups used, unless otherwise stated, are branched and unbranched alkyl groups with 1 to 4 carbon atoms which are bonded via an ester group. The alkyl groups are bonded directly to the carbonylcarbon of the ester group. The term —O—CO-alkyl-halogen group should be understood analogously. The group —O—CO—CF₃ denotes trifluoroacetate.

Within the scope of the present invention halogen denotes fluorine, chlorine, bromine or iodine. Unless otherwise stated, fluorine and bromine are the preferred halogens. The group CO denotes a carbonyl group.

By acid addition salts with pharmacologically acceptable acids of the compounds 1 are meant for example salts selected from among hydrochloride, hydrobromide, hydriodide, hydrosulphate, hydrophosphate, hydromethanesulphonate, hydronitrate, hydromaleate, hydroacetate, hydrobenzoate, hydrocitrate, hydrofumarate, hydrotartrate, hydroxalate, hydrosuccinate, hydrobenzoate and hydro-p-toluenesulphonate, preferably hydrochloride, hydrobromide, hydrosulphate, hydrophosphate, hydrofumarate and hydromethanesulphonate. Of the above-mentioned acid addition salts, the salts of hydrochloric acid, methanesulphonic acid, benzoic acid and acetic acid are particularly preferred according to the invention.

Indications

The present invention also relates to the use of therapeutically effective amounts of the active substances 1 for preparing a pharmaceutical composition also containing one or more, preferably one active substance 2 for the treatment of inflammatory and obstructive respiratory complaints, for inhibiting premature labour in midwifery (tocolysis), for restoring sinus rhythm in the heart in atrioventricular block, for correcting bradycardic heart rhythm disorders (antiarrhythmic), for treating circulatory shock (vasodilatation and increasing the heart volume) as well as for the treatment of skin irritations and inflammation.

In a preferred aspect the present invention relates to the use of therapeutically effective amounts of the active substances 1 for preparing a pharmaceutical composition also containing one or more, preferably one, active substance 2 for the treatment of respiratory complaints selected from the group comprising obstructive pulmonary diseases of various origins, pulmonary emphysema of various origins, restrictive pulmonary diseases, interstitial pulmonary diseases, cystic fibrosis, bronchitis of various origins, bronchiectasis, ARDS (adult respiratory distress syndrome) and all forms of pulmonary oedema.

Preferably the medicament combinations according to the invention are used as specified above for preparing a pharmaceutical composition for the treatment of obstructive pulmonary diseases selected from among bronchial asthma, paediatric asthma, severe asthma, acute asthma attacks, chronic bronchitis and COPD (chronic obstructive pulmonary disease), while it is particularly preferable according to the invention to use them for preparing a pharmaceutical composition for the treatment of bronchial asthma and COPD.

It is also preferable to use the medicament combinations according to the invention for preparing a pharmaceutical composition for the treatment of pulmonary emphysema which has its origins in COPD (chronic obstructive pulmonary disease) or α1-proteinase inhibitor deficiency.

It is also preferable to use the medicament combinations according to the invention for preparing a pharmaceutical composition for the treatment of restrictive pulmonary diseases selected from among allergic alveolitis, restrictive pulmonary diseases triggered by work-related noxious substances, such as asbestosis or silicosis, and restriction caused by lung tumours, such as for example lymphangiosis carcinomatosa, bronchoalveolar carcinoma and lymphomas.

It is also preferable to use the medicament combinations according to the invention for preparing a pharmaceutical composition for the treatment of interstitial pulmonary diseases selected from among pneumonia caused by infections, such as for example infection by viruses, bacteria, fungi, protozoa, helminths or other pathogens, pneumonitis caused by various factors, such as for example aspiration and left heart insufficiency, radiation-induced pneumonitis or fibrosis, collagenoses, such as for example lupus erythematodes, systemic sclerodermy or sarcoidosis, granulomatoses, such as for example Boeck's disease, idiopathic interstitial pneumonia or idiopathic pulmonary fibrosis (IPF). It is also preferable to use the medicament combinations according to the invention for preparing a pharmaceutical composition for the treatment of cystic fibrosis or mucoviscidosis.

It is also preferable to use the medicament combinations according to the invention for preparing a pharmaceutical composition for the treatment of bronchitis, such as for example bronchitis caused by bacterial or viral infection, allergic bronchitis and toxic bronchitis.

It is also preferable to use the medicament combinations according to the invention for preparing a pharmaceutical composition for the treatment of bronchiectasis.

It is also preferable to use the medicament combinations according to the invention for preparing a pharmaceutical composition for the treatment of ARDS (adult respiratory distress syndrome).

It is also preferable to use the medicament combinations according to the invention for preparing a pharmaceutical composition for the treatment of pulmonary oedema, for example toxic pulmonary oedema after aspiration or inhalation of toxic substances and foreign substances.

It is particularly preferable to use the compounds detailed above for preparing a pharmaceutical composition for the treatment of asthma or COPD. Also of particular importance is the above-mentioned use of medicament combinations according to the invention for preparing a pharmaceutical composition for once-a-day treatment of inflammatory and obstructive respiratory complaints, particularly for the once-a-day treatment of asthma or COPD.

Formulation

The present invention also relates to the use of therapeutically effective amounts of an active substance of formula 1 in combination with therapeutically effective amounts of an active substance 2 for preparing a pharmaceutical composition for the treatment of one of the above-mentioned diseases.

The present invention also relates to a process for treating one of the above-mentioned diseases, which is characterised in that therapeutically effective amounts of active substance of formula 1 are administered in combination with therapeutically effective amounts of active substance 2.

Within the scope of the medicament combinations according to the invention, for example, 0.1-1000 μg of a compound of formula 1 may be administered per single dose. Preferably, 1-500 μg, particularly preferably 3-100 μg of the compound of formula 1 are administered per single dose, while a dosage range of from 5-75 μg, preferably from 7-50 μg is preferred according to the invention. Particularly preferably, the pharmaceutical compositions according to the invention are administered in an amount such that 9-40 μg, particularly preferably 11-30 μg, more preferably 12-25 μg of the compound of formula 1 are administered per single dose. For example, and without restricting the present invention thereto, 5 μg, 7.5 μg, 10 μg, 12.5 μg, 15 μg, 17.5 μg, 20 μg, 22.5 μg, 25 μg, 27.5 μg, 30 μg, 32.5 μg, 35 μg, 37.5 μg, 40 μg, 42.5 μg, 45 μg, 47.5 μg, 50 μg, 52.5 μg, 55 μg, 57.5 μg, 60 μg, 62.5 μg, 65 μg, 67.5 μg, 70 μg, 72.5 μg or 75 μg of a compound of formula 1 may be administered per single dose.

The above-mentioned dosages relate to the compounds of formula 1 in the form of their free bases. If the compounds of formula 1 are administered in the form of their pharmaceutically acceptable acid addition salts, the skilled man can easily calculate the corresponding dosage ranges for the acid addition salts from the dosage ranges specified above, taking into account the molecular weight of the acids used. Particularly preferably, the compounds of formula 1 are administered in the above-mentioned dosage ranges in the form of the enantiomerically pure compounds, particularly preferably in the form of the R-enantiomers thereof.

If the compounds of formula 1 are administered in conjunction with an anticholinergic 2a, the amount of anticholinergic used will fluctuate considerably depending on the choice of active substance.

Without restricting the invention thereto, in the case of tiotropium 2a.1′ amounts of anticholinergic (2a.1′) may be administered such that each single dose contains 0.1-80 μg, preferably 0.5-60 μg, particularly preferably about 1-50 μg of 2a.1′. For example and without restricting the present invention thereto, 2.5 μg, 5 μg, 10 μg, 18 μg, 20 μg, 36 μg or 40 μg 2a.1′ may be administered per single dose. The corresponding amount of salt 2a.1 or of any hydrate or solvate used in each case can easily be calculated by the skilled man, depending on the choice of anion. If for example tiotropium bromide is used as the preferred tiotropium salt 2a.1 according to the invention, the amounts of the active substance 2a.1′ administered per single dose as specified by way of example hereinbefore correspond to the following amounts of 2a.1 administered per single dose: 3 μg, 6 μg, 12 μg, 21.7 μg, 24.1 μg, 43.3 μg and 48.1 μg of 2a.1. In the case of tiotropium 2a.1′ the dosages specified above are preferably administered once or twice a day, while administration once a day is particularly preferred according to the invention.

Without restricting the invention thereto, in the case of the cation 2a.2′ amounts of anticholinergic (2a.2′) may be administered such that each single dose contains 1-500 μg, preferably 5-300 μg, particularly preferably 15-200 μg 2a.2′. For example and without restricting the present invention thereto, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg or 200 μg of 2a.2′ may be administered per single dose. The corresponding amount of salt 2a.2 used in each case or of any hydrate or solvate used can easily be calculated by the skilled man, depending on the choice of anion. In the case of oxitropium 2a.2′ the dosages specified above are preferably administered one to four times a day, while administration two to three times a day is particularly preferred according to the invention.

Without restricting the invention thereto, in the case of the cation 2a.3′ amounts of anticholinergic (2a.3′) may be administered such that each single dose contains 1-500 μg, preferably 5-300 μg, particularly preferably 15-200 μg 2a.3′. For example and without restricting the present invention thereto, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg or 200 μg of 2a.3′ may be administered per single dose. The corresponding amount of salt 2a.3 used in each case or of any hydrate or solvate used can easily be calculated by the skilled man, depending on the choice of anion. In the case of flutropium 2a.3′ the dosages specified above are preferably administered one to four times a day, while administration two to three times a day is particularly preferred according to the invention.

Without restricting the invention thereto, in the case of the cation 2a.4′ amounts of anticholinergic (2a.4′) may be administered such that each single dose contains 1-500 μg, preferably 5-300 μg, particularly preferably 20-200 μg 2a.4′. For example and without restricting the present invention thereto, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg or 200 μg of 2a.4′ may be administered per single dose. The corresponding amount of salt 2a.4 used in each case or of any hydrate or solvate used can easily be calculated by the skilled man, depending on the choice of anion. In the case of ipratropium 2a.4′ the dosages specified above are preferably administered one to four times a day, while administration two to three times a day, more preferably three times a day, is particularly preferred according to the invention.

Without restricting the invention thereto, in the case of the cation 2a.5′ amounts of anticholinergic (2a.5′) may be administered such that each single dose contains 1-500 μg, preferably 5-300 μg, particularly preferably 15-200 μg. For example and without restricting the present invention thereto, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg or 200 μg of 2a.5′ may be administered per single dose. The corresponding amount of salt 2a.5 used in each case or of any hydrate or solvate used can easily be calculated by the skilled man, depending on the choice of anion. In the case of glycopyrronium 2a.5′ the dosages specified above are preferably administered one to four times a day, while administration two to three times a day is particularly preferred according to the invention.

Without restricting the invention thereto, in the case of the cation 2a.6′ amounts of anticholinergic (2a.6′) may be administered such that each single dose contains 1000-6500 μg, preferably 2000-6000 μg, particularly preferably 3000-5500 μg, particularly preferably 4000-5000 μg 2a.6′. For example and without restricting the present invention thereto, 3500 μg, 3750 μg, 4000 μg, 4250 μg, 4500 μg, 4750 μg, or 5000 μg of 2a.6′ may be administered per single dose. The corresponding amount of salt 2a.6 used in each case or of any hydrate or solvate used can easily be calculated by the skilled man, depending on the choice of anion. In the case of trospium 2a.6′ the dosages specified above are preferably administered one to four times a day, while administration two to three times a day is particularly preferred according to the invention.

Without restricting the invention thereto, in the case of the cation 2a.7′ amounts of anticholinergic (2a.7′) may be administered such that each single dose contains 50-1000 μg, preferably 100-800 μg, particularly preferably 200-700 μg, particularly preferably 300-600 μg 2a.7′. For example and without restricting the present invention thereto, 300 μg, 350 μg, 400 μg, 450 μg, 500 μg, 550 μg, or 600 μg of 2a.7′ may be administered per single dose. The corresponding amount of salt 2a.7 used in each case or of any hydrate or solvate used can easily be calculated by the skilled man, depending on the choice of anion. In the case of the cation 2a.7′ the dosages specified above are preferably administered one to three times a day, while administration once or twice a day, more preferably once a day, is particularly preferred according to the invention.

Without restricting the invention thereto, in the case of the cations 2a.9′ and 2a.10′, amounts of anticholinergic (2a.9′ or 2a.10′) may be administered such that each single dose contains 1-500 μg, preferably 5-300 μg, particularly preferably 15-200 μg 2a.9′ or 2a.10′. For example and without restricting the present invention thereto, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg or 200 μg of 2a.9′ or 2a.10′ may be administered per single dose. The corresponding amount of salt 2a.9′ or 2a.10′ or of any hydrate or solvate used in each case can easily be calculated by the skilled man, depending on the choice of anion. In the case of the cations 2a.9′ or 2a.10′ the dosages specified above are preferably administered one to three times a day, while administration once or twice a day, more preferably once a day, is particularly preferred according to the invention.

Without restricting the invention thereto, in the case of the cations 2a.11′ to 2a.13′ amounts of anticholinergic (2a.11′, 2a.12′ or 2a.13′) may be administered such that each single dose contains 1-500 μg, preferably 5-300 μg, particularly preferably 10-200 μg 2a.11′, 2a.12′ or 2a.13′. For example and without restricting the present invention thereto, 10 μg, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg or 200 μg of 2a.11′, 2a.12′ or 2a.13′ may be administered per single dose. The corresponding amount of salt 2a.11, 2a.12 or 2a.13 or of any hydrate or solvate used in each case can easily be calculated by the skilled man, depending on the choice of anion.

In the case of the cations 2a.11, 2a.12 or 2a.13 the dosages specified above are preferably administered one to three times a day, while administration once or twice a day, more preferably once a day, is particularly preferred according to the invention.

If the compounds of formula 1 are administered in combination with a PDE IV-inhibitor 2b, preferably about 1-10000 μg 2b are administered per single dose. Preferably, amounts of 2b are administered such that each single dose contains 10-5000 μg, preferably 50-2500 μg, particularly preferably 100-1000 μg of 2b. For example and without restricting the present invention thereto, 100 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg, 200 μg, 205 μg, 210 μg, 215 μg, 220 μg, 225 μg, 230 μg, 235 μg, 240 μg, 245 μg, 250 μg, 255 μg, 260 μg, 265 μg, 270 μg, 275 μg, 280 μg, 285 μg, 290 μg, 295 μg, 300 μg, 305 μg, 310 μg, 315 μg, 320 μg, 325 μg, 330 μg, 335 μg, 340 μg, 345 μg, 350 μg, 355 μg, 360 μg, 365 μg, 370 μg, 375 μg, 380 μg, 385 μg, 390 μg, 395 μg, 400 μg, 405 μg, 410 μg, 415 μg, 420 μg, 425 μg, 430 μg, 435 μg, 440 μg, 445 μg, 450 μg, 455 μg, 460 μg, 465 μg, 470 μg, 475 μg, 480 μg, 485 μg, 490 μg, 495 μg, 500 μg, 505 μg, 510 μg, 515 μg, 520 μg, 525 μg, 530 μg, 535 μg, 540 μg, 545 μg, 550 μg, 555 μg, 560 μg, 565 μg, 570 μg, 575 μg, 580 μg, 585 μg, 590 μg, 595 μg, 600 μg, 605 μg, 610 μg, 615 μg, 620 μg, 625 μg, 630 μg, 635 μg, 640 μg, 645 μg, 650 μg, 655 μg, 660 μg, 665 μg, 670 μg, 675 μg, 680 μg, 685 μg, 690 μg, 695 μg, 700 μg, 705 μg, 710 μg, 715 μg, 720 μg, 725 μg, 730 μg, 735 μg, 740 μg, 745 μg, 750 μg, 755 μg, 760 μg, 765 μg, 770 μg, 775 μg, 780 μg, 785 μg, 790 μg, 795 μg, 800 μg, 805 μg, 810 μg, 815 μg, 820 μg, 825 μg, 830 μg, 835 μg, 840 μg, 845 μg, 850 μg, 855 μg, 860 μg, 865 μg, 870 μg, 875 μg, 880 μg, 885 μg, 890 μg, 895 μg, 900 μg, 905 μg, 910 μg, 915 μg, 920 μg, 925 μg, 930 μg, 935 μg, 940 μg, 945 μg, 950 μg, 955 μg, 960 μg, 965 μg, 970 μg, 975 μg, 980 μg, 985 μg, 990 μg, 995 μg or 1000 μg of 2b may be administered per single dose. In the event that acid addition salts of 2b are used, the corresponding amount of salt used can easily be calculated by the skilled man from the values given hereinbefore, depending on the choice of acid.

If the compounds of formula 1 are administered in combination with a steroid 2c, preferably about 1-10000 μg of 2c are administered per single dose. Preferably, amounts of 2c are administered such that each single dose contains 5-5000 μg, preferably 5-2500 μg, particularly preferably 10-1000 μg of 2c. For example and without restricting the present invention thereto, 10 μg, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg, 200 μg, 205 μg, 210 μg, 215 μg, 220 μg, 225 μg, 230 μg, 235 μg, 240 μg, 245 μg, 250 μg, 255 μg, 260 μg, 265 μg, 270 μg, 275 μg, 280 μg, 285 μg, 290 μg, 295 μg, 300 μg, 305 μg, 310 μg, 315 μg, 320 μg, 325 μg, 330 μg, 335 μg, 340 μg, 345 μg, 350 μg, 355 μg, 360 μg, 365 μg, 370 μg, 375 μg, 380 μg, 385 μg, 390 μg, 395 μg, 400 μg, 405 μg, 410 μg, 415 μg, 420 μg, 425 μg, 430 μg, 435 μg, 440 μg, 445 μg, 450 μg, 455 μg, 460 μg, 465 μg, 470 μg, 475 μg, 480 μg, 485 μg, 490 μg, 495 μg, 500 μg, 505 μg, 510 μg, 515 μg, 520 μg, 525 μg, 530 μg, 535 μg, 540 μg, 545 μg, 550 μg, 555 μg, 560 μg, 565 μg, 570 μg, 575 μg, 580 μg, 585 μg, 590 μg, 595 μg, 600 μg, 605 μg, 610 μg, 615 μg, 620 μg, 625 μg, 630 μg, 635 μg, 640 μg, 645 μg, 650 μg, 655 μg, 660 μg, 665 μg, 670 μg, 675 μg, 680 μg, 685 μg, 690 μg, 695 μg, 700 μg, 705 μg, 710 μg, 715 μg, 720 μg, 725 μg, 730 μg, 735 μg, 740 μg, 745 μg, 750 μg, 755 μg, 760 μg, 765 μg, 770 μg, 775 μg, 780 μg, 785 μg, 790 μg, 795 μg, 800 μg, 805 μg, 810 μg, 815 μg, 820 μg, 825 μg, 830 μg, 835 μg, 840 μg, 845 μg, 850 μg, 855 μg, 860 μg, 865 μg, 870 μg, 875 μg, 880 μg, 885 μg, 890 μg, 895 μg, 900 μg, 905 μg, 910 μg, 915 μg, 920 μg, 925 μg, 930 μg, 935 μg, 940 μg, 945 μg, 950 μg, 955 μg, 960 μg, 965 μg, 970 μg, 975 μg, 980 μg, 985 μg, 990 μg, 995 μg or 1000 μg of 2c may be administered per single dose. In the event that salts or derivatives of 2c are used, the corresponding amount of salt/derivative used can easily be calculated by the skilled man from the values given hereinbefore, depending on the choice of salt/derivative.

If the compounds of formula 1 are administered in combination with an LTD4-antagonist 2d, preferably about 0.01-500 mg 2d are administered per single dose. Preferably, amounts of 2d are administered such that each single dose contains 0.1-250 mg, preferably 0.5-100 mg, particularly preferably 1-50 mg of 2d. For example and without restricting the present invention thereto, 1 mg, 2.5 mg, 5 mg, 5.5 mg, 7 mg, 7.5 mg, 10 mg, 12.5 mg, 15 mg, 17.5 mg, 20 mg, 22.5 mg, 25 mg, 27.5 mg, 30 mg, 32.5 mg, 35 mg, 37.5 mg, 40 mg, 42.5 mg, 45 mg, 47.5 mg or 50 mg of 2d may be administered per single dose. In the event that acid addition salts, salts or derivatives of 2d are used, the corresponding amount of salt/derivative used can easily be calculated by the skilled man from the values given hereinbefore, depending on the choice of salt/derivative.

If the compounds of formula 1 are administered in combination with an EGFR-inhibitor 2e, preferably about 100-15000 μg of 2e are administered per single dose. Preferably, amounts of 2e are administered such that each single dose contains 500-10000 μg, preferably 750-8000 μg, particularly preferably 1000-7000 μg of 2e. For example and without restricting the present invention thereto, 1000 μg, 1150 μg, 1200 μg, 1250 μg, 1300 μg, 1350 μg, 1400 μg, 1450 μg, 1500 μg, 1550 μg, 1600 μg, 1650 μg, 1700 μg, 1750 μg, 1800 μg, 1850 μg, 1900 μg, 1950 μg, 2000 μg, 2050 μg, 2100 μg, 2150 μg, 2200 μg, 2250 μg, 2300 μg, 2350 μg, 2400 μg, 2450 μg, 2500 μg, 2550 μg, 2600 μg, 2650 μg, 2700 μg, 2750 μg, 2800 μg, 2850 μg, 2900 μg, 2950 μg, 3000 μg, 3050 μg, 3100 μg, 3150 μg, 3200 μg, 3250 μg, 3300 μg, 3350 μg, 3400 μg, 3450 μg, 3500 μg, 3550 μg, 3600 μg, 3650 μg, 3700 μg, 3750 μg, 3800 μg, 3850 μg, 3900 μg, 3950 μg, 4000 μg, 4050 μg, 4100 μg, 4150 μg, 4200 μg, 4250 μg, 4300 μg, 4350 μg, 4400 μg, 4450 μg, 4500 μg, 4550 μg, 4600 μg, 4650 μg, 4700 μg, 4750 μg, 4800 μg, 4850 μg, 4900 μg, 4950 μg, 5000 μg, 5050 μg, 5100 μg, 5150 μg, 5200 μg, 5250 μg, 5300 μg, 5350 μg, 5400 μg, 5450 μg, 5500 μg, 5550 μg, 5600 μg, 5650 μg, 5700 μg, 5750 μg, 5800 μg, 5850 μg, 5900 μg, 5950 μg, 6000 μg, 6050 μg, 6100 μg, 6150 μg, 6200 μg, 6250 μg, 6300 μg, 6350 μg, 6400 μg, 6450 μg, 6500 μg, 6550 μg, 6600 μg, 6650 μg, 6700 μg, 6750 μg, 6800 μg, 6850 μg, 6900 μg, 6950 μg, or 7000 μg of 2e may be administered per single dose. In the event that acid addition salts of 2e are used, the corresponding amount of the salt used can easily be calculated by the skilled man from the values given hereinbefore, depending on the choice of acid.

The two active substance components 1 and 2 may be administered—together or separately—in each case by inhalation or by oral, parenteral or some other route, in known manner, in substantially conventional formulations such as for example plain or coated tablets, pills, granules, aerosols, syrups, emulsions, suspensions, powders and solutions, using inert, non-toxic, pharmaceutically suitable carriers or solvents.

Suitable preparations for administering the compounds of formula 1 and 2 include tablets, capsules, suppositories, solutions, powders, etc. The proportion of pharmaceutically active compound or compounds should be in the range from 0.05 to 90% by weight, preferably 0.1 to 50% by weight of the total composition. Suitable tablets may be obtained, for example, by mixing the active substance(s) with known excipients, for example inert diluents such as calcium carbonate, calcium phosphate or lactose, disintegrants such as corn starch or alginic acid, binders such as starch or gelatine, lubricants such as magnesium stearate or talc and/or agents for delaying release, such as carboxymethyl cellulose, cellulose acetate phthalate, or polyvinyl acetate. The tablets may also comprise several layers.

Coated tablets may be prepared accordingly by coating cores produced analogously to the tablets with substances normally used for tablet coatings, for example collidone or shellac, gum arabic, talc, titanium dioxide or sugar. To achieve delayed release or prevent incompatibilities the core may also consist of a number of layers. Similarly the tablet coating may consist of a number or layers to achieve delayed release, possibly using the excipients mentioned above for the tablets.

Syrups or elixirs containing the active substances or combinations of active substances according to the invention may additionally contain a sweetener such as saccharine, cyclamate, glycerol or sugar and a flavour enhancer, e.g. a flavouring such as vanillin or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates.

Solutions are prepared in the usual way, e.g. with the addition of isotonic agents, preservatives such as p-hydroxybenzoates, or stabilisers such as alkali metal salts of ethylenediamine tetraacetic acid, optionally using emulsifiers and/or dispersants, whilst if water is used as the diluent, for example, organic solvents may optionally be used as solvating agents or dissolving aids, and transferred into injection vials or ampoules or infusion bottles.

Capsules containing one or more active substances or combinations of active substances may for example be prepared by mixing the active substances with inert carriers such as lactose or sorbitol and packing them into gelatine capsules. Suitable suppositories may be made for example by mixing with carriers provided for this purpose, such as neutral fats or polyethyleneglycol or the derivatives thereof.

Excipients which may be used include, for example, water, pharmaceutically acceptable organic solvents such as paraffins (e.g. petroleum fractions), vegetable oils (e.g. groundnut or sesame oil), mono- or polyfunctional alcohols (e.g. ethanol or glycerol), carriers such as e.g. natural mineral powders (e.g. kaolins, clays, talc, chalk), synthetic mineral powders (e.g. highly dispersed silicic acid and silicates), sugars (e.g. cane sugar, lactose and glucose), emulsifiers (e.g. lignin, spent sulphite liquors, methylcellulose, starch and polyvinylpyrrolidone) and lubricants (e.g. magnesium stearate, talc, stearic acid and sodium lauryl sulphate).

For oral administration the tablets may, of course, contain, apart from the abovementioned carriers, additives such as sodium citrate, calcium carbonate and dicalcium phosphate together with various additional substances such as starch, preferably potato starch, gelatine and the like. Moreover, lubricants such as magnesium stearate, sodium lauryl sulphate and talc may be used at the same time for the tabletting process. In the case of aqueous suspensions the active substances may be combined with various flavour enhancers or colourings in addition to the excipients mentioned above.

Preferably, even when the two components 1 and 2 are administered separately, at least component 1 is administered by inhalation. If component 1 is administered by inhalation, when the two active substances are taken separately, component 2 may also be administered for example by oral or parenteral route using formulations conventional in the art such as plain or coated tablets, pills, granules, aerosols, syrups, emulsions, suspensions, powders and solutions, using inert, non-toxic, pharmaceutically suitable carriers or solvents.

Preferably, however, the medicament combinations according to the invention are administered by inhalation by means of a single preparation containing both active substances 1 and 2 or by means of separate preparations each containing only one of the active substances 1 and 2, suitable for administration by inhalation.

Inhalable preparations include inhalable powders, propellant-containing metered dose aerosols or propellant-free inhalable solutions. Inhalable powders according to the invention containing the combination of active substances 1 and 2 may consist of the active substances on their own or of a mixture of the active substances with physiologically acceptable excipients. Within the scope of the present invention, the term propellant-free inhalable solutions also includes concentrates or sterile inhalable solutions ready for use. The preparations according to the invention may contain the combination of active substances 1 and 2 either together in one formulation or in two separate formulations. These formulations which may be used within the scope of the present invention are described in more detail in the next part of the specification.

A) Inhalable Powder Containing the Combinations of Active Substances According to the Invention:

The inhalable powders according to the invention may contain 1 and 2 either on their own or in admixture with suitable physiologically acceptable excipients. If the active substances 1 and 2 are present in admixture with physiologically acceptable excipients, the following physiologically acceptable excipients may be used to prepare these inhalable powders according to the invention: monosaccharides (e.g. glucose or arabinose), disaccharides (e.g. lactose, saccharose, maltose, trehalose), oligo- and polysaccharides (e.g. dextrans), polyalcohols (e.g. sorbitol, mannitol, xylitol), salts (e.g. sodium chloride, calcium carbonate) or mixtures of these excipients with one another. Preferably, mono- or disaccharides are used, while the use of lactose, trehalose or glucose is preferred, particularly, but not exclusively, in the form of their hydrates.

Within the scope of the inhalable powders according to the invention the excipients have a maximum average particle size of up to 250 μm, preferably between 10 and 150 μm, most preferably between 15 and 80 μm. It may sometimes seem appropriate to add finer excipient fractions with an average particle size of 1 to 9 μm to the excipients mentioned above. These finer excipients are also selected from the group of possible excipients listed hereinbefore. Finally, in order to prepare the inhalable powders according to the invention, micronised active substance 1 and 2, preferably with an average particle size of 0.5 to 10 μm, more preferably from 1 to 6 μm, is added to the excipient mixture. Processes for producing the inhalable powders according to the invention by grinding and micronising and finally mixing the ingredients together are known from the prior art. The inhalable powders according to the invention may be prepared and administered either in the form of a single powder mixture which contains both 1 and 2 or in the form of separate inhalable powders which contain only 1 or 2.

The inhalable powders according to the invention may be administered using inhalers known from the prior art. Inhalable powders according to the invention which contain a physiologically acceptable excipient in addition to 1 and 2 may be administered, for example, by means of inhalers which deliver a single dose from a supply using a measuring chamber as described in U.S. Pat. No. 4,570,630A, or by other means as described in DE 36 25 685 A. The inhalable powders according to the invention which contain 1 and 2 optionally in conjunction with a physiologically acceptable excipient may be administered, for example, using the inhaler known by the name Turbohaler® or using inhalers as disclosed for example in EP 237507 A. Preferably, the inhalable powders according to the invention which contain physiologically acceptable excipients in addition to 1 and 2 are packed into capsules (to produce so-called inhalettes) which are used in inhalers as described, for example, in WO 94/28958.

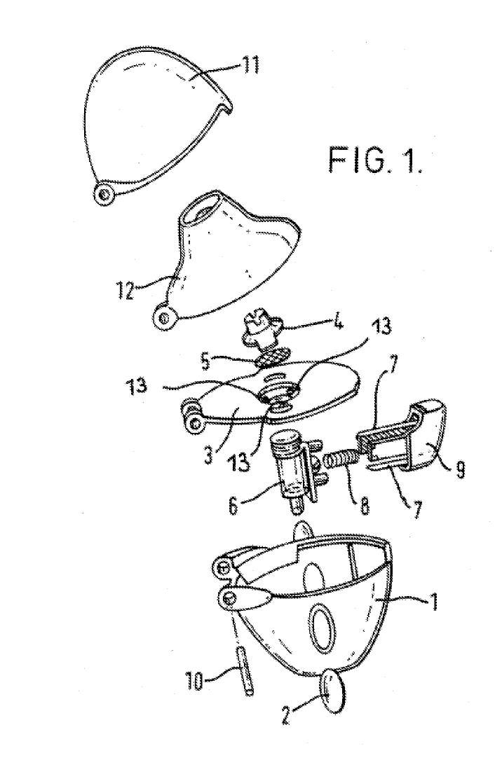

A particularly preferred inhaler for using the pharmaceutical combination according to the invention in inhalettes is shown in FIG. 1. This inhaler (Handihaler®) for inhaling powdered pharmaceutical compositions from capsules is characterised by a housing 1 containing two windows 2, a deck 3 in which there are air inlet ports and which is provided with a screen 5 secured by a screen housing 4, an inhalation chamber 6 connected to the deck 3 on which there is a push button 9 provided with two sharpened pins 7 and movable counter to a spring 8, and a mouthpiece 12 which is connected to the housing 1, the deck 3 and a cover 11 via a spindle 10 to enable it to be flipped open or shut, and air through-holes 13 for adjusting the flow resistance.

If the inhalable powders according to the invention are to be packaged in capsules, in accordance with the preferred method of administration described above, the capsules should preferably contain from 1 to 30 mg each. According to the invention they contain either together or separately the dosages per single dose specified for 1 and 2 hereinbefore.

B) Propellant Gas-Driven Inhalation Aerosols Containing the Combinations of Active Substances According to the Invention:

Inhalation aerosols containing propellant gas according to the invention may contain substances 1 and 2 dissolved in the propellant gas or in dispersed form. 1 and 2 may be present in separate formulations or in a single preparation, in which 1 and 2 are either both dissolved, both dispersed or only one component is dissolved and the other is dispersed. The propellant gases which may be used to prepare the inhalation aerosols according to the invention are known from the prior art. Suitable propellant gases are selected from among hydrocarbons such as n-propane, n-butane or isobutane and halohydrocarbons such as preferably chlorinated and fluorinated derivatives of methane, ethane, propane, butane, cyclopropane or cyclobutane. The propellant gases mentioned above may be used on their own or in mixtures thereof. Particularly preferred propellant gases are halogenated alkane derivatives selected from TG11, TG12, TG134a (1,1,1,2-tetrafluoroethane), TG227 (1,1,1,2,3,3,3-heptafluoropropane) and mixtures thereof, the propellant gases TG134a, TG227 and mixtures thereof being preferred.

The propellant-driven inhalation aerosols according to the invention may also contain other ingredients such as co-solvents, stabilisers, surfactants, antioxidants, lubricants and pH adjusters. All these ingredients are known in the art.

The inhalation aerosols containing propellant gas according to the invention may contain up to 5 wt.-% of active substance 1 and/or 2. Aerosols according to the invention contain, for example, 0.002 to 5 wt.-%, 0.01 to 3 wt.-%, 0.015 to 2 wt.-%, 0.1 to 2 wt.-%, 0.5 to 2 wt.-% or 0.5 to 1 wt.-% of active substance 1 and/or 2.

If the active substances 1 and/or 2 are present in dispersed form, the particles of active substance preferably have an average particle size of up to 10 μm, preferably from 0.1 to 6 μm, more preferably from 1 to 5 μm.

The propellant-driven inhalation aerosols according to the invention mentioned above may be administered using inhalers known in the art (MDIs=metered dose inhalers). Accordingly, in another aspect, the present invention relates to pharmaceutical compositions in the form of propellant-driven aerosols as hereinbefore described combined with one or more inhalers suitable for administering these aerosols. In addition, the present invention relates to inhalers which are characterised in that they contain the propellant gas-containing aerosols described above according to the invention.

The present invention also relates to cartridges which are fitted with a suitable valve and can be used in a suitable inhaler and which contain one of the above-mentioned propellant gas-containing inhalation aerosols according to the invention. Suitable cartridges and methods of filling these cartridges with the inhalable aerosols containing propellant gas according to the invention are known from the prior art.

C) Propellant-Free Inhalable Solutions or Suspensions Containing the Combinations of Active Substances 1 and 2 According to the Invention:

Propellant-free inhalable solutions according to the invention contain for example aqueous or alcoholic, preferably ethanolic solvents, possibly ethanolic solvents in admixture with aqueous solvents. In the case of aqueous/ethanolic solvent mixtures the relative proportion of ethanol to water is not restricted, but the maximum limit is up to 70 percent by volume, more particularly up to 60 percent by volume of ethanol. The remainder of the volume is made up of water. The solutions or suspensions containing 1 and 2, separately or together, are adjusted to a pH of 2 to 7, preferably 2 to 5, using suitable acids. The pH may be adjusted using acids selected from inorganic or organic acids. Examples of particularly suitable inorganic acids include hydrochloric acid, hydrobromic acid, nitric acid, sulphuric acid and/or phosphoric acid. Examples of particularly suitable organic acids include ascorbic acid, citric acid, malic acid, tartaric acid, maleic acid, succinic acid, fumaric acid, acetic acid, formic acid and/or propionic acid, etc. Preferred inorganic acids are hydrochloric acid and sulphuric acid. It is also possible to use the acids which have already formed an acid addition salt with one of the active substances. Of the organic acids, ascorbic acid, fumaric acid and citric acid are preferred. If desired, mixtures of the above acids may also be used, particularly in the case of acids which have other properties in addition to their acidifying qualities, e.g. as flavourings, antioxidants or complexing agents, such as citric acid or ascorbic acid, for example. According to the invention, it is particularly preferred to use hydrochloric acid to adjust the pH.

According to the invention, the addition of edetic acid (EDTA) or one of the known salts thereof, sodium edetate, as stabiliser or complexing agent is unnecessary in the present formulation. Other embodiments may contain this compound or these compounds. In a preferred embodiment the content based on sodium edetate is less than 100 mg/100 ml, preferably less than 50 mg/100 ml, more preferably less than 20 mg/100 ml. Generally, inhalable solutions in which the content of sodium edetate is from 0 to 10 mg/100 ml are preferred.

Co-solvents and/or other excipients may be added to the propellant-free inhalable solutions according to the invention. Preferred co-solvents are those which contain hydroxyl groups or other polar groups, e.g. alcohols—particularly isopropyl alcohol, glycols—particularly propyleneglycol, polyethyleneglycol, polypropyleneglycol, glycolether, glycerol, polyoxyethylene alcohols and polyoxyethylene fatty acid esters. The terms excipients and additives in this context denote any pharmacologically acceptable substance which is not an active substance but which can be formulated with the active substance or substances in the pharmacologically suitable solvent in order to improve the qualitative properties of the active substance formulation. Preferably, these substances have no pharmacological effect or, in connection with the desired therapy, no appreciable or at least no undesirable pharmacological effect. The excipients and additives include, for example, surfactants such as soya lecithin, oleic acid, sorbitan esters, such as polysorbates, polyvinylpyrrolidone, other stabilisers, complexing agents, antioxidants and/or preservatives which guarantee or prolong the shelf life of the finished pharmaceutical formulation, flavourings, vitamins and/or other additives known in the art. The additives also include pharmacologically acceptable salts such as sodium chloride as isotonic agents.

The preferred excipients include antioxidants such as ascorbic acid, for example, provided that it has not already been used to adjust the pH, vitamin A, vitamin E, tocopherols and similar vitamins and provitamins occurring in the human body.

Preservatives may be used to protect the formulation from contamination with pathogens. Suitable preservatives are those which are known in the art, particularly cetyl pyridinium chloride, benzalkonium chloride or benzoic acid or benzoates such as sodium benzoate in the concentration known from the prior art. The preservatives mentioned above are preferably present in concentrations of up to 50 mg/100 ml, more preferably between 5 and 20 mg/100 ml.

Preferred formulations contain, in addition to the solvent water and the combination of active substances 1 and 2, only benzalkonium chloride and sodium edetate. In another preferred embodiment, no sodium edetate is present.

The propellant-free inhalable solutions according to the invention are administered in particular using inhalers of the kind which are capable of nebulising a small amount of a liquid formulation in the therapeutic dose within a few seconds to produce an aerosol suitable for therapeutic inhalation. Within the scope of the present invention, preferred inhalers are those in which a quantity of less than 100 μL, preferably less than 50 μL, more preferably between 10 and 30 μL of active substance solution can be nebulised in preferably one spray action to form an aerosol with an average particle size of less than 20 μm, preferably less than 10 μm, such that the inhalable part of the aerosol corresponds to the therapeutically effective quantity.

An apparatus of this kind for propellant-free delivery of a metered quantity of a liquid pharmaceutical composition for inhalation is described for example in International Patent Application WO 91/14468 and also in WO 97/12687 (cf. in particular FIGS. 6a and 6b). The nebulisers (devices) described therein are known by the name Respimat®.

The above-mentioned examples of the active substances 2 are known in the art. The compounds of formula 1 by contrast are not known in the art.

The examples of synthesis described hereinafter serve to illustrate possible methods of synthesising the new compounds of formula 1. However, they are intended only as examples of procedures as an illustration of the invention without restricting the invention to the subject-matter described by way of example.

### EXAMPLES

### Example 1

### N-(5-{2-[1,1-dimethyl-3-(4-methyl-2-oxo-4H-benzo[d][1,3]oxazin-1-yl)-propylamino]-1-hydroxy-ethyl}-2-hydroxy-phenyl)-methanesulphonamide

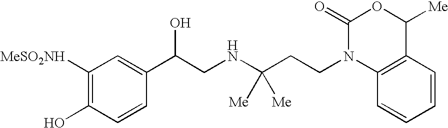

The compound is known from EP 43940. The individual diastereomers of this embodiment may be obtained by common methods known in the art.

### Example 2

### N-(5-{2-[1,1-dimethyl-3-(2-oxo-4H-benzo[d][1,3]oxazin-1-yl)-propylamino]-1-hydroxy-ethyl}-2-hydroxy-phenyl)-methanesulphonamide

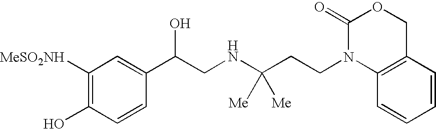

The compound is known from EP 43940. The (R)- and (S)-enantiomers of this embodiment may be obtained by common methods known in the art.

### Example 3

### N-(5-{2-[3-(4-ethyl-2-oxo-4H-benzo[d][1,3]oxazin-1-yl)-1,1-dimethyl-propylamino]-1-hydroxy-ethyl}-2-hydroxy-phenyl)-methanesulphonamide

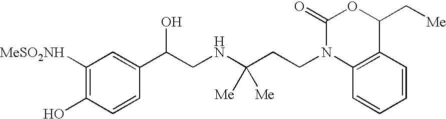

The compound is known from EP 43940. The individual diastereomers of this embodiment may be obtained by common methods known in the art.

### Example 4

### N-(5-{2-[3-(4,4-dimethyl-2-oxo-4H-benzo[d][1,3]oxazin-1-yl)-1,1-dimethyl-propylamino]-1-hydroxy-ethyl}-2-hydroxy-phenyl)-methanesulphonamide

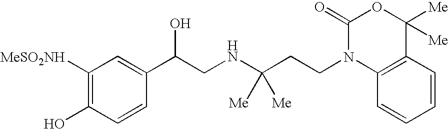

The compound is known from EP 43940. The (R)- and (S)-enantiomers of this embodiment may be obtained by common methods known in the art.

### Example 5

### N-(2-hydroxy-5-{1-hydroxy-2-[3-(6-hydroxy-4,4-dimethyl-2-oxo-4H-benzo[d][1,3]oxazin-1-yl)-1,1-dimethyl-propylamino]-ethyl}-phenyl)-methanesulphonamide

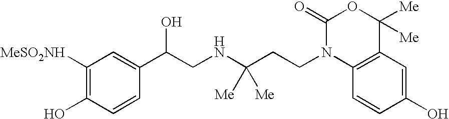

The compound is known from EP 43940. The (R)- and (S)-enantiomers of this embodiment may be obtained by common methods known in the art.

### Example 6

### N-(2-hydroxy-5-{1-hydroxy-2-[3-(6-methoxy-4,4-dimethyl-2-oxo-4H-benzo[d][1,3]oxazin-1-yl)-1,1-dimethyl-propylamino]-ethyl}-phenyl)-methanesulphonamide

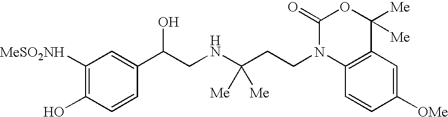

The compound is known from EP 43940. The (R)- and (S)-enantiomers of this embodiment may be obtained by common methods known in the art.

The examples of synthesis described below serve to illustrate new compounds according to the invention in more detail. However, they are intended only as examples of procedures to illustrate the invention without restricting it to the subject matter described in an exemplifying capacity hereinafter.

HPLC method (method A): Symmetry C18 (Waters): 3.5 μm; 4.6×150 mm; column temperature: 20° C.; gradient: acetonitrile/phosphate buffer (pH 7) 20:80→80:20 in 30 minutes; flow: 1.0 mL/min; detection at 220 and 254 nm.

Synthesis of Intermediate Products 1-8

### Intermediate Product 1

### 1-(3-amino-3-methyl-butyl)-4,4-dipropyl-1,4-dihydro-benzo[d][1,3]oxazin-2-one

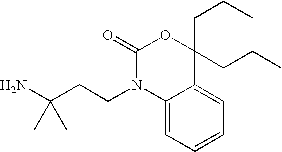

a) 4-(2-amino-phenyl)-heptan-4-ol: 90 mL (180.0 mmol) propylmagnesium chloride (2 M in ether) are added dropwise to a solution of 7.00 mL (54.0 mmol) methyl anthranilate in abs. THF (70 mL) at 0° C. within 30 minutes. The mixture is stirred for one hour at ambient temperature and then combined with 100 mL of 3 molar aqueous ammonium chloride solution and ethyl acetate. The phases are separated and the aqueous phase is exhaustively extracted with ethyl acetate. The combined organic phases are washed with potassium hydrogen carbonate solution and saturated sodium chloride solution and dried on sodium sulphate. The crude product is used in the next reaction step without further purification. Yield: 6.70 g (60%).

b) tert-butyl {3-[2-(1-hydroxy-1-propyl-butyl)-phenylamino]-1,1-dimethyl-propyl}-carbamate: 1.40 g (22.27 mmol) sodium cyanoborohydride are added to a solution of 3.10 g (14.05 mmol) 4-(2-amino-phenyl)-heptan-4-ol and 3.60 g (17.88 mmol) tert-butyl (1,1-dimethyl-3-oxo-propyl)-carbamate in methanol (40 mL) and acetic acid (6 mL). The mixture is stirred for 16 hours at ambient temperature, diluted with ethyl acetate, washed with 0.5 molar potassium hydrogen sulphate solution and saturated sodium chloride solution, dried on sodium sulphate and evaporated down in vacuo. The crude product is used in the next reaction step without further purification. Yield: 6.00 g (quantitative yield).

c) tert-butyl [1,1-dimethyl-3-(2-oxo-4,4-dipropyl-4H-benzo[d][1,3]oxazin-1-yl)-propyl]-carbamate: 8.85 mL (16.81 mmol) phosgene solution (20 wt. % in toluene) are slowly added dropwise at 0° C. to a solution of 6.00 g (15.28 mmol) tert-butyl {3-[2-(1-hydroxy-1-propyl-butyl)-phenylamino]-1,1-dimethyl-propyl}-carbamate and 5.32 mL (38.21 mmol) triethylamine in abs. THF (80 mL). The mixture is stirred for 2 hours at ambient temperature, diluted with ethyl acetate, combined with ice and made basic with saturated aqueous ammonia solution. The aqueous phase is exhaustively extracted with ethyl acetate and the combined organic phases are washed with saturated sodium chloride solution, dried on sodium sulphate and evaporated down in vacuo. After column chromatography (silica gel, cyclohexane/ethyl acetate=6:1) the product is obtained as a yellow oil. Yield: 4.57 g (71%).

d) 1-(3-amino-3-methyl-butyl)-4,4-dipropyl-1,4-dihydro-benzo[d][1,3]oxazin-2-one: A solution of 4.20 g (10.03 mmol) tert-butyl [1,1-dimethyl-3-(2-oxo-4,4-dipropyl-4H-benzo[d][1,3]oxazin-1-yl)-propyl]-carbamate in 35 mL formic acid is stirred for 24 hours at ambient temperature and then poured onto ice. The aqueous phase is made basic with saturated aqueous ammonia solution and exhaustively extracted with ethyl acetate. The combined organic extracts are washed with sodium chloride solution, dried on sodium sulphate and evaporated down in vacuo. The residue is taken up in ethyl acetate (50 mL) and combined with 4 mL hydrochloric acid in ethyl acetate (saturated). The solution is evaporated down and twice mixed with a little ethanol and evaporated down in vacuo. Trituration of the residue with diisopropylether yields the product as the hygroscopic hydrochloride salt. Yield: 2.60 g (73%).

### Intermediate Product 2

### 1-(3-amino-3-methyl-butyl)-4,4-diethyl-7-fluoro-1,4-dihydro-benzo[d][1,3]oxazin-2-one

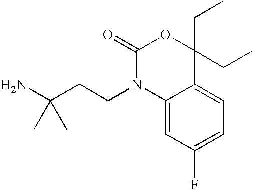

a) 3-(2-amino-4-fluoro-phenyl)-pentan-3-ol: The product is obtained analogously to intermediate product 1a by reacting methyl 2-amino-4-fluoro-benzoate and ethylmagnesium bromide in dichloromethane at −78° C. with heating to ambient temperature. Yield: 4.1 g (99%).

b) tert-butyl {3-[2-(1-ethyl-1-hydroxy-propyl)-5-fluoro-phenylamino]-1,1-dimethyl-propyl}-carbamate: The product is obtained analogously to intermediate product 1b starting from 3-(2-amino-4-fluoro-phenyl)-pentan-3-ol and tert-butyl (1,1-dimethyl-3-oxo-propyl)-carbamate. The crude product is purified by column chromatography (silica gel, dichloromethane/methanol=100:0→98:2). Yield: 7.70 g (99%).

c) tert-butyl [3-(4,4-diethyl-7-fluoro-2-oxo-4H-benzo[d][1,3]oxazin-1-yl)-1,1-dimethyl-propyl]-carbamate: The product is obtained analogously to intermediate product 1c starting from tert-butyl {3-[2-(1-ethyl-1-hydroxy-propyl)-5-fluoro-phenylamino]-1,1-dimethyl-propyl}-carbamate. Yield: 4.20 g (51%).

d) 1-(3-amino-3-methyl-butyl)-4,4-diethyl-7-fluoro-1,4-dihydro-benzo[d][1,3]oxazin-2-one: The product is prepared analogously to intermediate product 1d starting from tert-butyl [3-(4,4-diethyl-7-fluoro-2-oxo-4H-benzo[d][1,3]oxazin-1-yl)-1,1-dimethyl-propyl]-carbamate as the free base.

Yield: 2.90 g (96%); ESI-MS: [M+H]⁺=309.

### Intermediate Product 3

### 1-(3-amino-3-methyl-butyl)-spiro(cyclopropyl-1,4′-2H-3′, 1′-benzoxazin)-2′-one

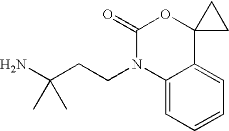

a) 1-(2-dibenzylamino-phenyl)-cyclopropanol: 2.45 mL (8.4 mmol) titanium tetraisopropoxide are slowly added dropwise at ambient temperature to a solution of 18.5 g (55.8 mmol) methyl 2-dibenzylamino-benzoate in 150 mL THF. After one hour's stirring 40.9 mL (122.7 mmol) ethylmagnesium bromide (3 M in diethyl ether) are added. The mixture is stirred for one hour, another 4 mL of 3 molar ethylmagnesium bromide solution are added and the mixture is stirred for 2 hours. The reaction mixture is combined with saturated ammonium chloride solution and extracted with ethyl acetate. The aqueous phase is combined with 1 molar hydrochloric acid until a clear solution is obtained and extracted with ethyl acetate. The combined organic phases are washed with sodium hydrogen carbonate solution and sodium chloride solution, dried on sodium sulphate and evaporated down. The residue is purified by chromatography (hexane/ethyl acetate=20:1). Yellow oil. Yield: 10.0 g (54%).

b) 1-(2-amino-phenyl)-cyclopropanol: 9.90 g (30.1 mmol) 1-(2-dibenzylamino-phenyl)-cyclopropanol are dissolved in 70 mL methanol and hydrogenated in the presence of 1 g palladium on charcoal (10%) at 3 bar hydrogen pressure. The catalyst is removed by suction filtering, the filtrate is evaporated down and the residue is purified by chromatography (silica gel; cyclohexane/ethyl acetate=5:1). White solid. Yield: 1.80 g (40%).

c) tert-butyl {3-[2-(1-hydroxy-cyclopropyl)-phenylamino]-1,1-dimethyl-propyl}-carbamate: Prepared analogously to the method described for intermediate product 1b from 1.77 g (11.86 mmol) 1-(2-amino-phenyl)-cyclopropanol and 3.15 g (15.66 mmol) tert-butyl (1,1-dimethyl-3-oxo-propyl)-carbamate. The crude product obtained is purified by column chromatography (silica gel, cyclohexane/ethyl acetate 4:1). Yellow oil. Yield: 2.60 g.

d) tert-butyl {1,1-dimethyl-3-[spiro(cyclopropyl-1,4′-2H-3′,1′-benzoxazin)-2′-oxo-1-yl]-propyl}-carbamate: The product is obtained analogously to intermediate product 1c starting from 2.60 g (7.74 mmol) tert-butyl {3-[2-(1-hydroxy-cyclopropyl)-phenylamino]-1,1-dimethyl-propyl}-carbamate. A difference here is that there is no purification by column chromatography. Yellow oil. Yield: 2.60 g.

e) 1-(3-amino-3-methyl-butyl)-spiro(cyclopropyl-1,4′-2H-3′,1′-benzoxazin)-2′-one: Obtained analogously to the method described for Intermediate 1d by reacting 3.10 g (8.60 mmol) tert-butyl {1,1-dimethyl-3-[spiro(cyclopropyl-1,4′-2H-3′,1′-benzoxazin)-2′-oxo-1-yl]-propyl}-carbamate and 30 mL formic acid. Yellow oil. Yield: 2.10 g (94%).

### Intermediate Product 4

### 1-(3-amino-3-methyl-butyl)-4,4-diethyl-1,4-dihydro-benzo[d][1,3]oxazin-2-one

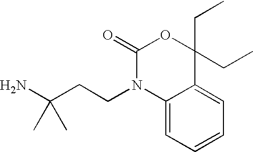

a) 3-(2-amino-phenyl)-pentan-3-ol: 100 mL of a 3 molar ethylmagnesium bromide solution in diethyl ether are added dropwise at −40° C. to a solution of 7.77 mL (60 mmol) 2-amino-methylbenzoic acid in 130 mL THF. The mixture is stirred overnight with heating to ambient temperature, combined with saturated ammonium chloride solution, acidified with 1 molar hydrochloric acid and extracted with ethyl acetate. The combined organic phases are extracted with water, dried on sodium sulphate and evaporated down. Dark red oil, which crystallises out and is further reacted directly. Yield: 10.9 g; mass spectroscopy: [M+H]⁺=180.

b) tert-butyl {3-[2-(1-ethyl-1-hydroxy-propyl)-phenylamino]-1,1-dimethyl-propyl}-carbamate: 3.16 g (47.7 mmol) sodium cyanoborohydride are added at ambient temperature to 5.70 g (31.8 mmol) 3-(2-amino-phenyl)-pentan-3-ol and 2.63 mL (47.7 mmol) acetic acid in 18 mL methanol. Then a solution of 7.04 g (35 mmol) tert-butyl (1,1-dimethyl-3-oxo-propyl)-carbamate in 18 mL methanol is slowly added dropwise. After the addition has ended the mixture is stirred for four hours, combined with 1 molar hydrochloric acid (development of gas) and then made basic with aqueous ammonia solution. It is extracted with ethyl acetate and the combined organic phases are washed with sodium chloride solution, dried on sodium sulphate and freed from the solvent. The residue is purified by column chromatography (silica gel, dichloromethane/methanol gradient with 0.1% ammonia). Yellow oil. Yield: 4.25 g (37%); mass spectroscopy: [M+H]⁺=365.

c) tert-butyl [3-(4,4-diethyl-2-oxo-4H-benzo[d][1,3]oxazin-1-yl)-1,1-dimethyl-propyl]-carbamate: 2.91 g (9.6 mmol) triphosgene are added at 0 to 5° C. to a solution of 3.50 g (9.6 mmol) tert-butyl {3-[2-(1-ethyl-1-hydroxy-propyl)-phenylamino]-1,1-dimethyl-propyl}-carbamate and 3.37 mL (24 mmol) triethylamine in 35 mL THF. The mixture is left overnight at ambient temperature with stirring and the precipitate formed is suction filtered. The filtrate is evaporated down and the oil remaining is further reacted directly.

Yield: 3.33 g; mass spectroscopy: [M+H]⁺=391.

d) 1-(3-amino-3-methyl-butyl)-4,4-diethyl-1,4-dihydro-benzo[d][1,3]oxazin-2-one: 25 mL trifluoroacetic acid are added dropwise, while being cooled with the ice bath, to a solution of 3.20 g tert-butyl [3-(4,4-diethyl-2-oxo-4H-benzo[d][1,3]oxazin-1-yl)-1,1-dimethyl-propyl]-carbamate (approx. 75%) in 25 mL dichloromethane. The mixture is stirred for 2 hours at ambient temperature, the solvents are distilled off and the acid residues are eliminated by repeated codistillation with toluene. To liberate the free base the residue is combined with 1 molar sodium hydroxide solution and extracted with ethyl acetate. The organic phases are dried on sodium sulphate and evaporated down. The free base is dissolved in 8 mL methanol and combined with ethereal hydrochloric acid. It is stirred overnight and the precipitate formed is suction filtered and washed with diethyl ether. Yield: 2.15 g (hydrochloride); mass spectroscopy: [M+H]⁺=291.

### Intermediate Product 5

### 1-(3-amino-3-methyl-butyl)-spiro(cyclohexane-1,4′-2H-3′,1′-benzoxazin)-2′-one

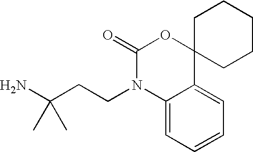

a) 1-(2-nitro-phenyl)-cyclohexanol: 40.16 mL (80.32 mmol) phenylmagnesium chloride (2 M in THF) are added dropwise at −50° C. under nitrogen to a solution of 20.0 g (80.32 mmol) 2-nitro-iodobenzene in 150 mL THF. After 15 minutes stirring 9.98 mL (96.30 mmol) cyclohexanone are quickly added. The reaction mixture is heated to ambient temperature, stirred for two hours and combined with ammonium chloride solution. The aqueous phase is separated off and exhaustively extracted with ethyl acetate. The combined organic phases are washed with sodium chloride solution, dried on sodium sulphate and evaporated down. Column chromatography (silica gel, hexane/ethyl acetate=20:1) yields the product as a brownish oil. Yield: 5.20 g (29%); R_{f}=0.26 (silica gel, hexane/ethyl acetate=10:1); ESI-MS: [M+H—H₂O]⁺=204.

b) 1-(2-amino-phenyl)-cyclohexanol: 5.20 g (16.45 mmol) 1-(2-nitro-phenyl)-cyclohexanol in 70 mL ethanol are hydrogenated for 4 hours in the presence of Raney nickel at ambient temperature and 3 bar hydrogen pressure. The catalyst is filtered off through Celite and the filtrate is evaporated down in vacuo. The residue is precipitated from hexane. Yield: 1.53 g (49%); R_{f}=0.38 (silica gel, hexane/ethyl acetate=4:1); ESI-MS: [M+H—H₂O]+=174.

c) tert-butyl {3-[2-(1-hydroxy-cyclohexyl)-phenylamino]-1,1-dimethyl-propyl}-carbamate: The compound is obtained analogously to intermediate product 1b from 1-(2-amino-phenyl)-cyclohexanol and tert-butyl (1,1-dimethyl-3-oxo-propyl)-carbamate. Column chromatography (silica gel, hexane/ethyl acetate=7:1) yields the product in the form of a colourless oil. Yield: 2.65 g (66%); R_{f}=0.50 (silica gel, hexane/ethyl acetate=4:1).

d) tert-butyl {1,1-dimethyl-3-[spiro(cyclohexane-1,4′-2H-3′, 1′-benzoxazin)-2′-oxo-1-yl]-propyl}-carbamate: Prepared analogously to intermediate product 1c from tert-butyl {3-[2-(1-hydroxy-cyclohexyl)-phenylamino]-1,1-dimethyl-propyl}-carbamate. Yield: 2.60 g (92%); R_{f}=0.38 (silica gel, hexane/ethyl acetate 4:1).

e) 1-(3-amino-3-methyl-butyl)-spiro(cyclohexane-1,4′-2H-3′,1′-benzoxazin)-2′-one: Prepared analogously to intermediate product 1d from tert-butyl [1,1-dimethyl-3-(spiro(cyclohexane-1,4′-2H-3′,1′-benzoxazin)-2′-oxo-1-yl)-propyl]-carbamate. Yield: 1.80 g (92%); R_{f}=0.10 (silica gel, dichloromethane/methanol/ammonia=95:5:0.5); ESI-MS: [M+H]⁺=303.

### Intermediate Product 6

### 1-(3-amino-3-methyl-butyl)-4,4-diethyl-8-methoxy-1,4-dihydro-benzo[d][1,3]oxazin-2-one

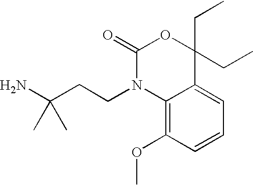

a) 3-(2-amino-3-methoxy-phenyl)-pentan-3-ol: The product is obtained analogously to intermediate product 1a by reacting methyl 2-amino-3-methoxy-benzoate and ethylmagnesium bromide in dichloromethane at −78° C.→RT. Yield: 5.20 g (92%); HPLC-MS: Rₜ=12.85 min. (method A); ESI-MS: [M+H]⁺=210.

b) tert-butyl {3-[2-(1-ethyl-1-hydroxy-propyl)-6-methoxy-phenylamino]-1,1-dimethyl-propyl}-carbamate: The product is obtained analogously to intermediate product 1b starting from 3-(2-amino-3-methoxy-phenyl)-pentan-3-ol and tert-butyl (1,1-dimethyl-3-oxo-propyl)-carbamate. The crude product is purified by column chromatography (silica gel, cyclohexane/ethyl acetate=4:1). Yield: 4.60 g (47%).

c) tert-butyl [3-(4,4-diethyl-8-methoxy-2-oxo-4H-benzo[d][1,3]oxazin-1-yl)-1,1-dimethyl-propyl]-carbamate: The product is obtained analogously to intermediate product 1c starting from tert-butyl {3-[2-(1-ethyl-1-hydroxy-propyl)-6-methoxy-phenylamino]-1,1-dimethyl-propyl}-carbamate. Yield: 4.60 g (94%).

d) 1-(3-amino-3-methyl-butyl)-4,4-diethyl-8-methoxy-1,4-dihydro-benzo[d][1,3]oxazin-2-one: The product is obtained analogously to intermediate product 1d starting from tert-butyl [3-(4,4-diethyl-8-methoxy-2-oxo-4H-benzo[d][1,3]oxazin-1-yl)-1,1-dimethyl-propyl]-carbamate as a free base. Yield: 3.00 g (93%); ESI-MS: [M+H]⁺=321.

### Intermediate Product 7

### 1-(3-amino-3-methyl-butyl)-4,4-diethyl-6-fluoro-1,4-dihydro-benzo[d][1,3]oxazin-2-one

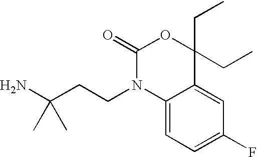

a) 3-(2-amino-5-fluoro-phenyl)-pentan-3-ol: Prepared analogously to intermediate product 1a from methyl 2-amino-5-fluoro-benzoate and ethylmagnesium bromide. The product obtained is purified by chromatography (silica gel, cyclohexane/ethyl acetate=8:1). Yield: 6.00 g (74%).

b) tert-butyl {3-[2-(1-ethyl-1-hydroxy-propyl)-4-fluoro-phenylamino]-1,1-dimethyl-propyl}-carbamate: The product is obtained analogously to intermediate product 1b starting from 3-(2-amino-5-fluoro-phenyl)-pentan-3-ol and tert-butyl (1,1-dimethyl-3-oxo-propyl)-carbamate. The crude product is purified by column chromatography (silica gel, hexane/ethyl acetate=6:1→2:1). Yield: 4.50 g (41%).

c) tert-butyl [3-(4,4-diethyl-6-fluoro-2-oxo-4H-benzo[d][1,3]oxazin-1-yl)-1,1-dimethyl-propyl]-carbamate: Prepared analogously to intermediate product 1c from tert-butyl {3-[2-(1-ethyl-1-hydroxy-propyl)-4-fluoro-phenylamino]-1,1-dimethyl-propyl}-carbamate. A difference here is that there is no purification by column chromatography. Colourless oil. Yield: 4.8 g.

d) 1-(3-amino-3-methyl-butyl)-4,4-diethyl-6-fluoro-1,4-dihydro-benzo[d][1,3]oxazin-2-one: The target compound is prepared as a free base analogously to intermediate product 1d from tert-butyl [3-(4,4-diethyl-6-fluoro-2-oxo-4H-benzo[d][1,3]oxazin-1-yl)-1,1-dimethyl-propyl]-carbamate. Yield: 3.00 g (99%).

### Intermediate Product 8

### 1-(3-amino-3-methyl-butyl)-4,4-diethyl-6-methoxy-1,4-dihydro-benzo[d][1,3]oxazin-2-one

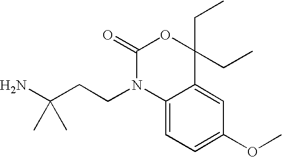

a) 3-(2-amino-5-methoxy-phenyl)-pentan-3-ol: the product is obtained by reacting 4.00 g (22 mmol) methyl 2-amino-5-methoxy-benzoate with 5 equivalents ethylmagnesium bromide in dichloromethane at −78° C.->RT. Brown oil. Yield: 4.47 g (97%).

b) tert-butyl {3-[2-(1-ethyl-1-hydroxy-propyl)-4-methoxy-phenylamino]-1,1-dimethyl-propyl}-carbamate: Prepared analogously to intermediate product 1b from 4.45 g (21 mmol) 3-(2-amino-5-methoxy-phenyl)-pentan-3-ol and 5.66 g (28 mmol) tert-butyl (1,1-dimethyl-3-oxo-propyl)-carbamate. Brown oil. Yield: 6.00 g (72%).

c) tert-butyl [3-(4,4-diethyl-6-methoxy-2-oxo-4H-benzo[d][1,3]oxazin-1-yl)-1,1-dimethyl-propyl]-carbamate: The product is prepared analogously to intermediate product 1c from 6.00 g (15.2 mmol) tert-butyl {3-[2-(1-ethyl-1-hydroxy-propyl)-4-methoxy-phenylamino]-1,1-dimethyl-propyl}-carbamate. Yellow oil. Yield: 3.10 g (48%).

d) 1-(3-amino-3-methyl-butyl)-4,4-diethyl-6-methoxy-1,4-dihydro-benzo[d][1,3]oxazin-2-one: Prepared analogously to intermediate product 1d from 3.10 g (8.5 mmol) tert-butyl [3-(4,4-diethyl-6-methoxy-2-oxo-4H-benzo[d][1,3]oxazin-1-yl)-1,1-dimethyl-propyl]-carbamate. The product is isolated as the free base and not converted into a hydrochloride salt. Yellow oil. Yield: 2.20 g (98%).

### Example 7

### N-(5-{2-[1,1-dimethyl-3-(2-oxo-4,4-dipropyl-4H-benzo[d][1,3]oxazin-1-yl)-propylamino]-1-hydroxy-ethyl}-2-hydroxy-phenyl)-methanesulphonamide

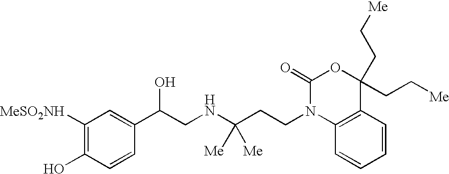

a) N-(2-benzyloxy-5-{2-[1,1-dimethyl-3-(2-oxo-4,4-dipropyl-4H-benzo[d][1,3]oxazin-1-yl)-propylamino]-1-hydroxy-ethyl}-phenyl)-methanesulphonamide: 86 μl (0.619 mmol) triethylamine are added at ambient temperature under a nitrogen atmosphere to a solution of 200 mg (0.564 mmol) 1-(3-amino-3-methyl-butyl)-4,4-dipropyl-1,4-dihydro-benzo[d][1,3]oxazin-2-one hydrochloride in 5 mL THF. The mixture is stirred for 30 minutes, 218 mg (0.575 mmol) N-[2-benzyloxy-5-(2-ethoxy-2-hydroxy-acetyl)-phenyl]-methanesulphonamide are added and the mixture is stirred for a further 2 hours at ambient temperature. The mixture is cooled to 10° C., combined with 51 mg (2.34 mmol) lithium borohydride, heated to ambient temperature and stirred for one hour. It is cooled to 10° C. again and diluted with 15 mL water and 20 mL dichloromethane. The aqueous phase is separated off and extracted with dichloromethane. The combined organic phases are dried on sodium sulphate and evaporated down in vacuo. The residue is dissolved in 8 mL ethyl acetate and acidified to pH 2 by the addition of saturated hydrochloric acid in ethyl acetate. The precipitate formed is filtered off, washed with ethyl acetate and evaporated down. Yield: 260 mg (67%, hydrochloride), HPLC: Rₜ=19.8 minutes (method A).

b) N-(5-{2-[1,1-dimethyl-3-(2-oxo-4,4-dipropyl-4H-benzo[d][1,3]oxazin-1-yl)-propylamino]-1-hydroxy-ethyl}-2-hydroxy-phenyl)-methanesulphonamide: 260 mg (0.386 mmol) N-(2-benzyloxy-5-{2-[1,1-dimethyl-3-(2-oxo-4,4-dipropyl-4H-benzo[d][1,3]oxazin-1-yl)-propylamino]-1-hydroxy-ethyl}-phenyl)-methanesulphonamide hydrochloride in 8 mL methanol are hydrogenated in the presence of 26 mg palladium on charcoal (10%) at ambient temperature. The catalyst is filtered off through Celite and washed with methanol. The filtrate is evaporated down in vacuo and the residue is stirred into diethyl ether. Yield: 120 mg (53%, hydrochloride); mass spectroscopy: [M+H]⁺=548; HPLC: Rₜ=14.7 minutes (method A).

The (R)- and (S)-enantiomers of this embodiment may be obtained by common methods known in the art. The (R)-enantiomer of this embodiment is of particular importance according to the invention.

### Example 8

### N-[5-(2-{1,1-dimethyl-3-[spiro(cyclohexane-1,4′-2H-3′,1′-benzoxazin)-2′-oxo-1-yl]-propylamino}-1-hydroxy-ethyl)-2-hydroxy-phenyl]-methanesulphonamide

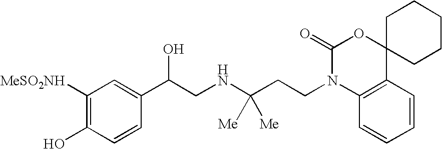

a) N-[2-benzyloxy-5-(2-{3-[spiro(cyclohexane-1,4′-2H-3′,1′-benzoxazin)-2′-oxo-1-yl]-1,1-dimethyl-propylamino}-1-hydroxy-ethyl]-phenyl]-methanesulphonamide: Prepared analogously to the process described for Example 7a from 250 mg (0.66 mmol) N-[2-benzyloxy-5-(2-ethoxy-2-hydroxy-acetyl)-phenyl]-methanesulphonamide and 200 mg (0.66 mmol) 1-(3-amino-3-methyl-butyl)-spiro(cyclohexane-1,4′-2H-3′,1′-benzoxazin)-2′-one. A difference here is that the product obtained as the hydrochloride is also purified by chromatography (silica gel, dichloromethane/methanol=50:1).

Yield: 190 mg (46%), HPLC: Rₜ=17.8 minutes (method A).

b) N-[5-(2-{1,1-dimethyl-3-[spiro(cyclohexane-1,4′-2H-3′,1′-benzoxazin)-2′-oxo-1-yl]-propylamino}-1-hydroxy-ethyl)-2-hydroxy-phenyl]-methanesulphonamide: 190 mg (0.31 mmol) N-[2-benzyloxy-5-(2-{3-[spiro(cyclohexane-1,4′-2H-3′,1′-benzoxazin)-2′-oxo-1-yl]-1,1-dimethyl-propylamino}-1-hydroxy-ethyl]-phenyl]-methanesulphonamide are hydrogenated analogously to Example 7b. After separation of the catalyst the filtrate is freed from the solvent, combined with 8 mL ethyl acetate and acidified to pH 2 by the addition of hydrochloric acid in ethyl acetate. The solvent is distilled off and the residue is stirred in diethyl ether and filtered. Yield: 40 mg (23%, hydrochloride); mass spectroscopy: [M+H]⁺=532; HPLC: Rₜ=11.8 minutes (method A).

The (R)- and (S)-enantiomers of this embodiment may be obtained by common methods known in the art. Particular importance attaches to the (R)-enantiomer of this embodiment according to the invention.

### Example 9

### N-[5-(2-{1,1-dimethyl-3-[spiro(cyclopropyl-1,4′-2H-3′,1′-benzoxazin)-2′-oxo-1-yl]-propylamino}-1-hydroxy-ethyl)-2-hydroxy-phenyl]-methanesulphonamide

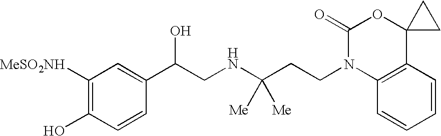

a) N-[2-benzyloxy-5-(2-{3-[spiro(cyclopropyl-1,4′-2H-3′,1′-benzoxazin)-2′-oxo-1-yl]-1,1-dimethyl-propylamino}-1-hydroxy-ethyl]-phenyl]-methanesulphonamide: 292 mg (0.77 mmol) N-[2-benzyloxy-5-(2-ethoxy-2-hydroxy-acetyl)-phenyl]-methanesulphonamide and 200 mg (0.77 mmol) 1-(3-amino-3-methyl-butyl)-spiro(cyclopropyl-1,4′-2H-3′,1′-benzoxazin)-2′-one are reacted and worked up analogously to Example 7a. The crude product is combined with 8 mL ethyl acetate and acidified to pH 2 with hydrochloric acid in ethyl acetate. The solvent is distilled off and the residue is stirred in diethyl ether. White solid. Yield: 400 mg (84%, hydrochloride), HPLC: Rₜ=15.2 minutes (method A).

b) N-[5-(2-{1,1-dimethyl-3-[spiro(cyclopropyl-1,4′-2H-3′,1′-benzoxazin)-2′-oxo-1-yl]-propylamino}-1-hydroxy-ethyl)-2-hydroxy-phenyl]-methanesulphonamide: the product is prepared analogously to Example 1b from 400 mg (0.65 mmol) N-[2-benzyloxy-5-(2-{3-[spiro(cyclopropyl-1,4′-2H-3′,1′-benzoxazin)-2′-oxo-1-yl]-1,1-dimethyl-propylamino}-1-hydroxy-ethyl]-phenyl]-methanesulphonamide hydrochloride. Yield: 230 mg (67%, hydrochloride); mass spectroscopy: [M+H]⁺=490; HPLC: Rₜ=8.9 minutes (method A).

The (R)- and (S)-enantiomers of this embodiment may be obtained by common methods known in the art. Particular importance attaches to the (R)-enantiomer of this embodiment according to the invention.

### Example 10

### N-(5-{2-[3-(4,4-diethyl-2-oxo-4H-benzo[d][1,3]oxazin-1-yl)-1,1-dimethyl-propylamino]-1-hydroxy-ethyl}-2-hydroxy-phenyl)-methanesulphonamide

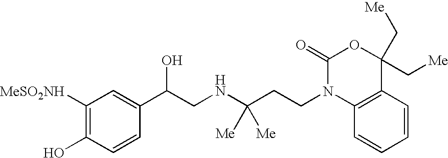

379 mg (1 mmol) N-[2-benzyloxy-5-(2-ethoxy-2-hydroxy-acetyl)-phenyl]-methanesulphonamide and 290 mg (1 mmol) 1-(3-amino-3-methyl-butyl)-4,4-diethyl-1,4-dihydro-benzo[d][1,3]oxazin-2-on are suspended in 5 mL ethanol and heated to 70° C. The resulting solution is stirred for one hour at 70° C. and then cooled to ambient temperature. After the addition of 113 mg (3 mmol) sodium borohydride the mixture is stirred for 3 hours at ambient temperature, combined with 0.7 mL saturated potassium carbonate solution and stirred for a further 30 minutes. The mixture is filtered through aluminium oxide (basic), washed repeatedly with dichloromethane/methanol (15:1) and evaporated down. The crude product thus obtained is purified by chromatography (dichloromethane with 0-10% methanol/ammonia=9:1). The benzylether thus obtained is dissolved in 10 mL methanol and hydrogenated with palladium on charcoal as catalyst at 1 bar hydrogen pressure. Then the catalyst is filtered off and the filtrate is evaporated down. White solid. Yield: 338 mg (65% over 2 steps); mass spectroscopy: [M+H]⁺=520.

The (R)- and (S)-enantiomers of this embodiment may be obtained by common methods known in the art. Particular importance attaches to the (R)-enantiomer of this embodiment according to the invention. The angle of rotation of (R)-N-(5-{2-[3-(4,4-diethyl-2-oxo-4H-benzo[d][1,3]oxazin-1-yl)-1,1-dimethyl-propylamino]-1-hydroxy-ethyl}-2-hydroxy-phenyl)-methanesulphonamide hydrochloride (cocrystallised with a molecule of acetone) is −28.8° (c=1%, in methanol at 20° C.).

### Example 11

### N-(5-{2-[3-(4,4-diethyl-6-fluoro-2-oxo-4H-benzo[d][1,3]oxazin-1-yl)-1,1-dimethyl-propylamino]-1-hydroxy-ethyl}-2-hydroxy-phenyl)-methanesulphonamide

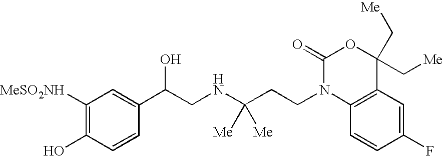

a) N-(2-benzyloxy-5-{2-[3-(4,4-diethyl-6-fluoro-2-oxo-4H-benzo[d][1,3]oxazin-1-yl)-1,1-dimethyl-propylamino]-1-hydroxy-ethyl}-phenyl)-methanesulphonamide: Reaction of 246 mg (0.65 mmol) N-[2-benzyloxy-5-(2-ethoxy-2-hydroxy-acetyl)-phenyl]-methanesulphonamide and 200 mg (0.65 mmol) 1-(3-amino-3-methyl-butyl)-4,4-diethyl-6-fluoro-1,4-dihydro-benzo[D][1,3]oxazin-2-one analogously to Example 7a. One difference is that the preparation of the hydrochloride is omitted. Instead, the free base is purified by chromatography (reverse phase, acetonitrile/water gradient with 0.1% trifluoroacetic acid).

Yield: 180 mg (trifluoroacetate), HPLC: Rₜ=17.4 minutes (method A).

b) N-(5-{2-[3-(4,4-diethyl-6-fluoro-2-oxo-4H-benzo[d][1,3]oxazin-1-yl)-1,1-dimethyl-propylamino]-1-hydroxy-ethyl}-2-hydroxy-phenyl)-methanesulphonamide: 175 mg of —N-(2-benzyloxy-5-{2-[3-(4,4-diethyl-6-fluoro-2-oxo-4H-benzo[d][1,3]oxazin-1-yl)-1,1-dimethyl-propylamino]-1-hydroxy-ethyl}-phenyl)-methanesulphonamide trifluoroacetate in 9 mL methanol are hydrogenated in the presence of 40 mg Raney nickel at ambient temperature and 3 bar hydrogen pressure. The catalyst is filtered off and the filtrate is freed from the solvent. White solid.

Yield: 131 mg (trifluoroacetate); mass spectroscopy: [M+H]⁺=538.

The (R)- and (S)-enantiomers of this embodiment may be obtained by common methods known in the art. Particular importance attaches to the (R)-enantiomer of this embodiment according to the invention.

### Example 12

### N-(5-{2-[3-(4,4-diethyl-7-fluoro-2-oxo-4H-benzo[d][1,3]oxazin-1-yl)-1,1-dimethyl-propylamino]-1-hydroxy-ethyl}-2-hydroxy-phenyl)-methanesulphonamide

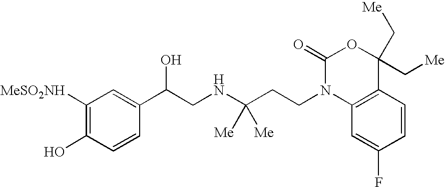

a) N-(2-benzyloxy-5-{2-[3-(4,4-diethyl-7-fluoro-2-oxo-4H-benzo[d][1,3]oxazin-1-yl)-1,1-dimethyl-propylamino]-1-hydroxy-ethyl}-phenyl)-methanesulphonamide: 246 mg (0.65 mmol) N-[2-benzyloxy-5-(2-ethoxy-2-hydroxy-acetyl)-phenyl]-methanesulphonamide and 200 mg (0.65 mmol) 1-(3-amino-3-methyl-butyl)-4,4-diethyl-7-fluoro-1,4-dihydro-benzo[d][1,3]oxazin-2-one are reacted and worked up analogously to Example 7a. A difference is that the production of the hydrochloride is omitted and the free base is purified by chromatography (reverse phase, acetonitrile/water gradient with 0.1% trifluoroacetic acid).

Yield: 220 mg (trifluoroacetate), HPLC: Rₜ=17.7 minutes (method A).

b) N-(5-{2-[3-(4,4-diethyl-7-fluoro-2-oxo-4H-benzo[d][1,3]oxazin-1-yl)-1,1-dimethyl-propylamino]-1-hydroxy-ethyl}-2-hydroxy-phenyl)-methanesulphonamide: Prepared analogously to Example 11b from 210 mg of N-(2-benzyloxy-5-{2-[3-(4,4-diethyl-7-fluoro-2-oxo-4H-benzo[d][1,3]oxazin-1-yl)-1,1-dimethyl-propylamino]-1-hydroxy-ethyl}-phenyl)-methanesulphonamide trifluoroacetate. Grey solid.

Yield: 154 mg (trifluoroacetate); mass spectroscopy: [M+H]⁺=538.

The (R)- and (S)-enantiomers of this embodiment may be obtained by common methods known in the art. Particular importance attaches to the (R)-enantiomer of this embodiment according to the invention.

### Example 13

### N-(5-{2-[3-(4,4-diethyl-8-methoxy-2-oxo-4H-benzo[d][1,3]oxazin-1-yl)-1,1-dimethyl-propylamino]-1-hydroxy-ethyl}-2-hydroxy-phenyl)-methanesulphonamide

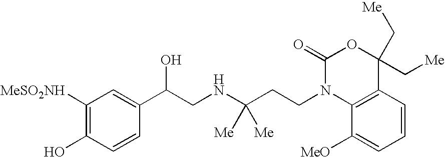

a) N-(2-benzyloxy-5-{2-[3-(4,4-diethyl-8-methoxy-2-oxo-4H-benzo[d][1,3]oxazin-1-yl)-1,1-dimethyl-propylamino]-1-hydroxy-ethyl}-phenyl)-methanesulphonamide: reaction of 237 mg (0.625 mmol) N-[2-benzyloxy-5-(2-ethoxy-2-hydroxy-acetyl)-phenyl]-methanesulphonamide and 200 mg (0.624 mmol) 1-(3-amino-3-methyl-butyl)-4,4-diethyl-8-methoxy-1,4-dihydro-benzo[d][1,3]oxazin-2-one analogously to Example 7a. The crude product is dissolved in ethyl acetate and acidified to pH 2 with hydrochloric acid in ethyl acetate. The solvent is distilled off and the residue is stirred in diethyl ether. Then the hydrochloride thus obtained (330 mg) is further purified by chromatography.

Yield: 90 mg (trifluoroacetate), HPLC: Rₜ=17.6 minutes (method A).

b) N-(5-{2-[3-(4,4-diethyl-8-methoxy-2-oxo-4H-benzo[d][1,3]oxazin-1-yl)-1,1-dimethyl-propylamino]-1-hydroxy-ethyl}-2-hydroxy-phenyl)-methanesulphonamide: 80 mg (0.118 mmol) N-(2-benzyloxy-5-{2-[3-(4,4-diethyl-8-methoxy-2-oxo-4H-benzo[d][1,3]oxazin-1-yl)-1,1-dimethyl-propylamino]-1-hydroxy-ethyl}-phenyl)-methanesulphonamide trifluoroacetate are hydrogenated analogously to Example 11b. Beige solid. Yield: 70 mg (trifluoroacetate); mass spectroscopy: [M+H]⁺=550.

The (R)- and (S)-enantiomers of this embodiment may be obtained by common methods known in the art. Particular importance attaches to the (R)-enantiomer of this embodiment according to the invention.

### Example 14

### N-(5-{2-[3-(4,4-diethyl-6-methoxy-2-oxo-4H-benzo[d][1,3]oxazin-1-yl)-1,1-dimethyl-propylamino]-1-hydroxy-ethyl}-2-hydroxy-phenyl)-methanesulphonamide

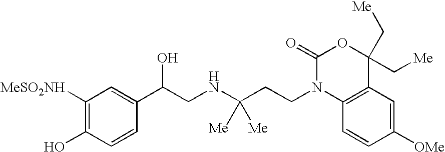

a) N-(2-benzyloxy-5-{2-[3-(4,4-diethyl-6-methoxy-2-oxo-4H-benzo[d][1,3]oxazin-1-yl)-1,1-dimethyl-propylamino]-1-hydroxy-ethyl}-phenyl)-methanesulphonamide: 235 mg (0.619 mmol) N-[2-benzyloxy-5-(2-ethoxy-2-hydroxy-acetyl)-phenyl]-methanesulphonamide and 200 mg (0.624 mmol) 1-(3-amino-3-methyl-butyl)-4,4-diethyl-6-methoxy-1,4-dihydro-benzo[d][1,3]oxazin-2-one are reacted analogously to Example 7a. One difference is that the crude product is not precipitated as the hydrochloride, but purified by chromatography (reverse phase, acetonitrile/water gradient with 0.1% trifluoroacetic acid).

Yield: 150 mg (trifluoroacetate), HPLC: Rₜ=16.9 minutes (method A).

b) N-(5-{2-[3-(4,4-diethyl-6-methoxy-2-oxo-4H-benzo[d][1,3]oxazin-1-yl)-1,1-dimethyl-propylamino]-1-hydroxy-ethyl}-2-hydroxy-phenyl)-methanesulphonamide: The target compound is prepared from N-(2-benzyloxy-5-{2-[3-(4,4-diethyl-6-methoxy-2-oxo-4H-benzo[d][1,3]oxazin-1-yl)-1,1-dimethyl-propylamino]-1-hydroxy-ethyl}-phenyl)-methanesulphonamide trifluoroacetate analogously to Example 11b. Grey solid. Mass spectroscopy: [M+H]⁺=550.

The (R)- and (S)-enantiomers of this embodiment may be obtained by common methods known in the art. Particular importance attaches to the (R)-enantiomer of this embodiment according to the invention.

1. A pharmaceutical combination comprising one or more compounds of general formula 1 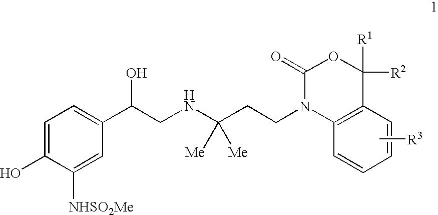
wherein
R¹ and R² independently of one another denote H, halogen or C₁₋₄-alkyl or together denote C₁₋₆-alkylene; and
R³ denotes H, halogen, OH, C₁₋₄-alkyl or O—C₁₋₄-alkyl;
and at least one further active substance 2.

2. The pharmaceutical combination according to claim 1 wherein said active substance 2 comprises one or more compounds selected from the categories of the anticholinergics (2a), PDE-IV-inhibitors (2b), steroids (2c), LTD4-antagonists (2d) and EGFR-inhibitors (2e).

3. The pharmaceutical combination according to claim 1, wherein
R¹ and R² which may be identical or different, denote hydrogen, fluorine, chlorine, methyl, ethyl, propyl, butyl or together denote —CH₂—CH₂, —CH₂—CH₂—CH₂, —CH₂—CH₂—CH₂—CH₂ or —CH₂—CH₂—CH₂—CH₂—CH₂—;
R³ denotes hydrogen, fluorine, chlorine, OH, methyl, ethyl, methoxy, or ethoxy.

4. The pharmaceutical combination according to claim 1, wherein
R¹ and R² which may be identical or different, denote ethyl, propyl or together denote —CH₂—CH₂, —CH₂—CH₂—CH₂, —CH₂—CH₂—CH₂—CH₂ or —CH₂—CH₂—CH₂—CH₂—CH₂—;
R³ denotes hydrogen, fluorine, OH, methyl or methoxy.

5. The pharmaceutical combination according to claim 1, wherein said one or more compounds of general formula 1 are in the form of the individual optical isomers, mixtures of the individual enantiomers or racemates.

6. The pharmaceutical combination according to claim 1, wherein said one or more compounds of general formula 1 are in the form of the acid addition salts with pharmacologically acceptable acids.

7. The pharmaceutical combination according to claim 1, wherein said one or more compounds of general formula 1 are in the form of the solvates thereof.

8. The pharmaceutical combination according to claim 1, wherein said one or more compounds of general formula 1 are in the form of the hydrates thereof.

9. The pharmaceutical combination according to claim 1, wherein said active substance 2 comprises an anticholinergic (2a).

10. The pharmaceutical combination according to claim 1, wherein said active substance 2 comprises a PDE IV-Inhibitor (2b).

11. The pharmaceutical combination according to claim 1, wherein said active substance 2 comprises a steroid (2c).

12. The pharmaceutical combination according to claim 1, wherein said active substance 2 comprises an LTD4-antagonist (2d).

13. The pharmaceutical combination according to claim 1, wherein said active substance 2 comprises an EGFR-inhibitor (2e).

14. The pharmaceutical combination according to claim 1, wherein said combination comprises:
a therapeutically effective amount of said one or more compounds of general formula 1;
a therapeutically effective amount of an anticholinergic (2a); and
a therapeutic amount of a PDE-IV inhibitor (2b).

15. The pharmaceutical combination according to claim 1, wherein said combination comprises:
a therapeutically effective amounts of said one or more compounds of general formula 1;
a therapeutically effective amount of an anticholinergic (2a); and
a therapeutic amount of a steroid (2c).

16. The pharmaceutical combination according to claim 1, wherein said combination comprises:
a therapeutically effective amount of said one or more compounds of general formula 1;
a therapeutically effective amount of an anticholinergic (2a); and
a therapeutic amount of an LTD4-antagonist (2d).

17. The pharmaceutical combination according to claim 1, wherein said combination comprises:
a therapeutically effective amount of said one or more compounds of general formula 1;
a therapeutically effective amount of an anticholinergic (2a); and
a therapeutic amounts of an EGFR inhibitor (2e).

18. The pharmaceutical combination according to claim 1, wherein said combination comprises:
a therapeutically effective amount of said one or more compounds of general formula 1;
a therapeutically effective amounts of a PDE-IV inhibitor (2b); and
a therapeutic amount of a steroid (2c).

19. The pharmaceutical combination according to claim 1, wherein said combination comprises:
a therapeutically effective amount of said one or more compounds of general formula 1;
a therapeutically effective amounts of a PDE-IV inhibitor (2b); and
a therapeutic amount of an LTD4-antagonist (2d).

20. The pharmaceutical combination according to claim 1, wherein said combination comprises:
a therapeutically effective amount of said one or more compounds of general formula 1;
a therapeutically effective amounts of a PDE-IV inhibitor (2b); and
a therapeutic amount of an EGFR inhibitor (2e).

21. The pharmaceutical combination according to claim 1, wherein said combination comprises:
a therapeutically effective amount of said one or more compounds of general formula 1;
a therapeutically effective amount of a steroid (2c); and
a therapeutic amount of an LTD4-antagonist (2d).

22. The pharmaceutical combination according to claim 1, wherein said combination comprises:
a therapeutically effective amount of said one or more compounds of general formula 1;
a therapeutically effective amount of a steroid (2c); and
a therapeutic amount of an EGFR inhibitor (2e).

23. The pharmaceutical combination according to claim 1, wherein said combination comprises:
a therapeutically effective amount of said one or more compounds of general formula 1;
a therapeutically effective amount of an LTD4-antagonist (2d); and
a therapeutic amount of an EGFR inhibitor (2e).

24. The pharmaceutical combination according to claim 1, wherein said combination comprises:
a therapeutically effective amount of said one or more compounds of general formula 1;
a therapeutically effective amount of said active substance 2; and
a pharmaceutically acceptable carrier.

25. The pharmaceutical combination according to claim 1, wherein said combination comprises:
a therapeutically effective amount of said one or more compounds of general formula 1; and
a therapeutically effective amount of said active substance 2;
wherein said combination contains no pharmaceutically acceptable carrier.

26. The pharmaceutical combination according to claim 1, wherein said combination is in the form of a formulation suitable for inhalation.

27. The pharmaceutical combination according to claim 26, wherein said combination is a preparation selected from an inhalable powder, a propellant-driven metered-dose aerosol and a propellant-free inhalable solution or suspension.

28. The pharmaceutical combination according to claim 27, wherein said preparation is an inhalable powder comprising 1 and 2 in admixture with suitable physiologically acceptable excipients selected from monosaccharides, disaccharides, oligo- and polysaccharides, polyalcohols, salts, or mixtures of these excipients with one another.

29. The pharmaceutical combination according to claim 27, wherein said preparation is a propellant-driven inhalable aerosol comprising 1 and 2 in dissolved or dispersed form.

30. The pharmaceutical combination according to claim 29, wherein said inhalable aerosol comprises as the propellant gas a hydrocarbon.

31. The pharmaceutical combination according to claim 30, wherein said hydrocarbon is n-propane, n-butane or isobutane or halohydrocarbons such as chlorinated and/or fluorinated derivatives of methane, ethane, propane, butane, cyclopropane or cyclobutane.

32. The pharmaceutical combination according to claim 30, wherein said propellant gas is TG11, TG12, TG134a or TG227, or a mixture thereof.

33. The pharmaceutical combination according to claim 32, wherein said propellant gas is TG134a or TG227, or a mixture thereof.

34. The pharmaceutical combination according to claim 27, wherein said preparation is a propellant-free inhalable solution or suspension comprising as solvent water, ethanol or a mixture of water and ethanol.

35. A method of treating inflammatory and obstructive respiratory complaints, for inhibiting premature labour in midwifery (tocolysis), for restoring sinus rhythm in the heart in atrioventricular block, for correcting bradycardic heart rhythm disorders (antiarrhythmic), for treating circulatory shock (vasodilatation and increasing the heart volume) or for treating skin irritations and inflammation comprising administering to a patient in need thereof a therapeutically effective amount of a pharmaceutical combination according to claim 1.

36. The method according to claim 35, wherein said respiratory complaints are obstructive pulmonary diseases of various origins, pulmonary emphysema of various origins, restrictive pulmonary diseases, interstitial pulmonary diseases, cystic fibrosis, bronchitis of various origins, bronchiectasis, ARDS (adult respiratory distress syndrome) or all forms of pulmonary oedema.

37. A method of treating inflammatory and obstructive respiratory complaints, for inhibiting premature labour in midwifery (tocolysis), for restoring sinus rhythm in the heart in atrioventricular block, for correcting bradycardic heart rhythm disorders (antiarrhythmic), for treating circulatory shock (vasodilatation and increasing the heart volume) or for treating skin irritations and inflammation comprising administering to a patient in need thereof a therapeutically effective amount of a compound of formula 1 according to one of claim 1 in combination with at least one additional active substance 2.
